(19) United States
(12) Patent Application Publication
(10) Pub. No.: US 2026/0256533 A1
(43) Pub. Date: Sep. 3, 2026

(54) VASCULAR ACCESS ROBOTIC SYSTEMS AND DEVICES

(71) Applicant: Hyperion Surgical, Inc., Dania Beach, FL (US)

(72) Inventor: Jonathan AZEVEDO, Fort Lauderdale, FL (US)

(73) Assignee: Hyperion Surgical, Inc., Dania Beach, FL (US)

(21) Appl. No.: 19/638,779

(22) Filed: Apr. 3, 2026

Related U.S. Application Data
(63) Continuation of application No. PCT/US2026/017131, filed on Feb. 27, 2026.
(60) Provisional application No. 63/765,114, filed on Feb. 28, 2025.
(60) Provisional application No. 63/881,562, filed on Sep. 14, 2025.
(60) Provisional application No. 63/913,817, filed on Nov. 7, 2025.

(51) Int. Cl. A61B 34/37; A61B 34/00; A61B 34/10; A61B 34/20; A61B 34/30; A61B 90/00; A61M 25/01

(52) U.S. Cl. CPC A61B 34/37; A61B 34/20; A61B 34/25; A61B 2034/107; A61B 2034/303; A61B 2090/364; A61B 2090/373; A61M 25/0113

(57) ABSTRACT

A robotic assistance device and method for vascular procedures using an imaging device to obtain multiple transverse and lateral views of a tissue region where a limited set of views is selected and displayed based on the location of a vessel within the tissue region. The systems and methods described herein can alter views altered in response to movement of vascular medical devices relative to the vessel. Variations of the systems and methods overlay one or more virtual images on the displayed views to assist the caregiver in operating the robotic assistance device to perform the procedure.

### CROSS-REFERENCE TO RELATED APPLICATIONS

This application is a continuation in part of PCT Application PCT/US2026/017131 filed Feb. 27, 2026, which is a non-provisional of U.S. Provisional Nos. 63/765,114, filed on Feb. 28, 2025; 63/881,562, filed on Sep. 14, 2025; and 63/913,817, filed on Nov. 7, 2025. The entirety of each of which is incorporated by reference.

### FIELD OF THE INVENTION

The present disclosure relates generally to robotic systems, devices, and methods for robotic-assisted vascular access. More specifically, the present disclosure relates to robotic systems, devices, and methods for positioning a needle, catheter, and/or guidewire into a blood vessel of a subject using imaging-assisted guidance.

### BACKGROUND OF THE INVENTION

Vascular catheterization, the process of positioning a medical catheter through tissue and into a blood vessel or other organ, is a common procedure that is required to perform a number of interventional medical procedures. Typically, a caregiver performs a catheterization using a needle, guidewire, and catheter. This procedure requires locating an approximate region on a surface of tissue and advancing a needle through this region and into a blood vessel, then inserting a guidewire through the needle such that the tip of the guidewire advances within a lumen of the blood vessel. Once the medical caregiver positions the guidewire with the desired length and/or location in the blood vessel, the caregiver can remove the needle and advance a catheter over the guidewire and into the blood vessel. Once the catheter is in the blood vessel, the guidewire can be removed, such that the catheter is used to facilitate subsequent procedures.

However, the reality is that such catheterization procedures can be difficult due to factors including, but not limited to, varying patient anatomy, caregiver experience/skill, the need to handle multiple components in addition to the needle, catheter, and guidewire, as well as increased risk to patients resulting from inadequate placement, multiple attempts, and/or delayed patient care.

In many cases, it is difficult and/or time-consuming to select a region on the skin that is adjacent to a blood vessel when the location of the vessel is not readily apparent on the skin. A caregiver may resort to massaging the skin, placing ice on the skin, or using other techniques to assist in locating an acceptable region. Moreover, in cases where the vessel is difficult to locate, a caregiver must keep their sight focused on a region of tissue, which can complicate the ability of the caregiver to manipulate the needle or other components. Adding a second person to assist the caregiver can increase labor costs as well as delays if the additional person is unavailable.

In addition, multiple attempts at needle placement can cause additional trauma apart from a delay in care. In some cases, the patient may be frail or young, where the trauma of multiple attempts is unacceptable. In some cases, failure to position an intravenous catheter in a peripheral vessel can require positioning of an invasive central line closer to the heart, which significantly increases infection and clotting risk to the patient.

One current attempt to address these problems is for the caregiver to use a hand-held ultrasound probe on tissue to locate the vessel and a target region on tissue. However, this technique requires the caregiver to manipulate the ultrasound probe on the surface of the skin and insert a needle while watching a screen that shows the ultrasound image. As a result, the caregiver's attention must be split between the patient and the screen. Moreover, the caregiver must keep the ultrasound probe in a stable, non-moving position during the procedure. Regardless, less-experienced caregivers may experience a learning curve when trying to locate a vessel within tissue using ultrasound images.

As noted above, in most healthcare environments, the availability of experienced caregivers is limited, and waiting for such caregivers can cause significant care delays, which increase risk to the patient and costs of the procedure.

In addition to the problems listed above, caregivers of any experience level are capable of committing human error. Such errors can lead to multiple placement attempts or extreme conditions, including, but not limited to, vessel perforation, pseudoaneurysm formation, hemorrhage, infection, etc. Therefore, human factors during the performance of catheterizations can lead to delays, errors, and inconsistencies in proper placement, leading to complications, increased medical expenses, and delay of care.

Accordingly, there is an unmet need to provide safety mechanisms and guidance to operators while inserting a needle and/or catheter into a patient to gain safe vascular access and to improve consistency, reduce procedure times, and reduce complications that may arise due to human errors. There is also an unmet need to provide a solution such that a less experienced or lower-cost technician can perform a successful catheterization procedure at an improved success rate.

### SUMMARY OF THE INVENTION

Variations of the present disclosure include a robotic system configured to assist a caregiver in a procedure of positioning one or more vascular medical devices into a vessel of a patient, the robotic system including: a control unit in communication with a processor; an arm member having a first end coupled to a base of the robotic system; a manipulation device coupled to a second end of the arm member, the manipulation device configured to both retain and selectively advance the one or more vascular medical devices; an imaging device coupled to the manipulation device opposite to the arm member and configured to obtain a plurality of transverse views and/or a plurality of lateral views of a tissue region of the patient; where the manipulation device is positionable independently from the base, such that the imaging device can be positioned adjacent to the tissue region; a video display coupled to the base and in communication with the processor, such that the processor is configured to select a first limited set of one or more of the plurality of transverse views and/or the plurality of lateral views for display on the video display, where the processor selects the first limited set, at least in part, based on a location of the vessel within the tissue region; and wherein the processor is configured to select a second limited set including one or more views from the plurality of transverse views and/or a plurality of lateral views to replace the first limited set for display on the video display, based upon movement of the one or more vascular medical devices relative to the vessel, and where at least one view of the second limited set is different than at least one view of the first limited set; and where the processor is further configured to provide at least one virtual image on the video display over the first limited set or over the second limited set to assist the caregiver during operation of the robotic system to deploy the one or more vascular medical devices.

Variations of the present disclosure include a robotic system, wherein the video display includes a primary focus region configured for displaying the first limited set or the second limited set, and a secondary focus region configured for displaying an alternate view of one of the plurality of transverse views and/or a plurality of lateral views.

Variations of the present disclosure include a robotic system, wherein the primary focus region is visually different from the secondary focus region by brightness, outlining, colorizing, scaling, or focus.

Variations of the present disclosure include a robotic system, wherein the processor is configured to switch the alternate view into the primary focus region upon advancement of the procedure.

Variations of the present disclosure include a robotic system, wherein the at least one virtual image includes a virtual cross-sectional image of a calculated centroid of a cross-section of the vessel displayed in at least one of the first limited set or the second limited set.

Variations of the present disclosure include a robotic system, wherein the at least one virtual image further includes a virtual path of the one or more vascular medical devices corresponding to an insertion trajectory of the one or more vascular medical devices.

Variations of the present disclosure include a robotic system, further including changing an appearance of the virtual cross-sectional image when the virtual path intersects the calculated centroid.

Variations of the present disclosure include a robotic system, wherein the video display includes a touch screen and displays one or more touch-screen commands to control the robotic system.

Variations of the present disclosure include a robotic system, wherein the one or more touch-screen commands alter an advancement of the one or more vascular medical devices.

Variations of the present disclosure include a robotic system, wherein the one or more touch-screen commands alter the advancement of the one or more vascular medical devices by altering a speed or an acceleration of the one or more vascular medical devices in the tissue region over a fixed distance.

Variations of the present disclosure include a robotic system, wherein the one or more touch-screen commands alter the advancement of the one or more vascular medical devices by vibrating the one or more vascular medical devices during movement in the tissue region.

Variations of the present disclosure include a robotic system, wherein the processor is configured to monitor a wall in the tissue region for tenting, and where the processor provides feedback to the caregiver based on a recoil of a portion of the wall.

Variations of the present disclosure include a robotic system, where the feedback includes an alert upon failure to detect the recoil.

Variations of the present disclosure include a robotic system, wherein the processor alters an advancement of the one or more vascular medical devices by automatically altering a speed or an acceleration of the one or more vascular medical devices in the tissue region over a fixed distance during the procedure.

Variations of the present disclosure include a robotic system, wherein the video display is configured to display a plurality of procedure options prior to starting the procedure, to permit the caregiver to select the procedure from the plurality of procedure options.

Variations of the present disclosure include a robotic system, wherein the procedure includes a plurality of sub-procedures, and displaying on the video display a sub-procedure screen for each of the plurality of sub-procedures such that the caregiver can select the sub-procedure screen for each of the plurality of sub-procedures in a sequential manner.

Variations of the present disclosure include a method of assisting a caregiver when using a robotic system to perform a procedure for positioning one or more vascular medical devices into a vessel of a patient, the method including: providing an imaging device of the robotic system adapted to be positioned by the caregiver adjacent to a surface of the patient, where the imaging device is configured to obtain a plurality of transverse views and/or a plurality of lateral views of a tissue region beneath the surface of the patient; displaying on a video display of the robotic system, a first limited set including one or more of the plurality of transverse views and/or the plurality of lateral views where selection of the first limited set is dependent, at least in part, on a location of the vessel within the tissue region; and altering the video display to display a second limited set including one or more views from the plurality of transverse views and/or a plurality of lateral views in response to movement of the one or more vascular medical devices relative to the vessel, where the second limited set is different than the first limited set; and providing at least one virtual image on the first limited set or the second limited set to assist the caregiver in operating the robotic system to insert the one or more vascular medical devices into the vessel.

Variations of the present disclosure include a method, wherein the at least one virtual image further includes a virtual path of the one or more vascular medical devices corresponding to a trajectory of the one or more vascular medical devices within the tissue region.

Variations of the present disclosure include a method, displaying a plurality of touch-screen commands on the video display where the plurality of touch-screen commands control the robotic system.

Variations of the present disclosure include a method, wherein the plurality of touch-screen commands includes altering an advancement of the one or more vascular medical devices.

Variations of the present disclosure include a method, wherein altering the advancement of the one or more vascular medical devices includes altering a speed or an acceleration of the one or more vascular medical devices in the tissue region over a fixed distance.

Variations of the present disclosure include a method, further including monitoring, using the robotic system, a wall in the tissue region for tenting and providing feedback to the caregiver upon detecting recoil of a portion of the wall.

Variations of the present disclosure include a method, further including providing feedback to the caregiver upon failure to detect recoil of the portion of the wall.

Variations of the present disclosure include a method, further including, prior to starting the procedure, displaying a plurality of procedure options on the video display to permit the caregiver to select the procedure.

Variations of the present disclosure include a robotic system configured to assist a caregiver in a procedure of positioning one or more vascular medical devices into a vessel of a patient, the robotic system including: a control unit in communication with a processor; an arm member having a first end coupled to a base of the robotic system; a manipulation device coupled to a second end of the arm member, the manipulation device configured to both retain and selectively advance the one or more vascular medical devices; an imaging device coupled to the manipulation device opposite to the arm member and configured to obtain a plurality of transverse views and/or a plurality of lateral views of a tissue region of the patient; where the manipulation device is positionable independently from the base, such that the imaging device can be positioned adjacent to the tissue region; a video display coupled to the base and in communication with the processor, such that the processor is configured to select a limited set of one or more of the plurality of transverse views and/or the plurality of lateral views for display on the video display, where the processor selects the limited set, at least in part, based on a location of the vessel within the tissue region; and where the processor is configured to change the limited set during the procedure in response to a position of or a change in the one or more vascular medical devices being manipulated by the robotic system; where the processor is further configured to provide at least one virtual image on the limited set to assist the caregiver in operating the robotic system to deploy the one or more vascular medical devices.

Variations of the present disclosure include a robotic system, wherein the at least one virtual image includes a virtual cross-sectional image of a calculated centroid of a cross-section of the vessel displayed in at least one of the limited set.

Variations of the present disclosure include a robotic system, further including changing an appearance of the virtual cross-sectional image when the virtual path intersects the calculated centroid.

Variations of the present disclosure include a robotic system, wherein the processor is configured to monitor a wall in the tissue region for tenting, and where the processor provides feedback to the caregiver based on a recoil of a portion of the wall.

Variations of the present disclosure include a method of assisting a caregiver when using a robotic system to perform a procedure for positioning one or more vascular medical devices into a vessel of a patient, the method including: obtaining a plurality of transverse views and/or a plurality of lateral views of a tissue region beneath a tissue surface of the patient from an imaging device of the robotic system, after the caregiver positions the imaging device adjacent to the tissue surface; displaying on a video display of the robotic system, a limited set including views from the plurality of transverse views and/or the plurality of lateral views, where selection of the limited set is dependent on a location of the vessel within the tissue region; and changing the limited set during the procedure in response to a position of or a change in the one or more vascular medical devices being manipulated by the robotic system; and providing at least one virtual image on the limited set to assist the caregiver in operating the robotic system to deploy the one or more vascular medical devices.

Variations of the present disclosure include a method, including: identifying a first portion of a target structure in a first transverse view captured by an imaging device; aligning a first marker with a centroid of the first portion; identifying a second portion of the target structure in a second transverse view captured by the imaging device; aligning a second marker with a centroid of the second portion; confirming that the first marker and the second marker are aligned with the centroid of the first portion and the centroid of the second portion, respectively; and in response to confirming that the centroid of the first portion and the centroid of the second portion are aligned, enabling actuation of one or more actuators to advance a needle, a catheter, and a guidewire into the target structure.

Variations of the present disclosure include a method, including: inserting, using a set of actuators, a guidewire, a needle, and a catheter into a target vessel of a patient, the guidewire, the needle, and the catheter being disposed within a cartridge; receiving a signal to retract at least the needle and the guidewire from the target vessel; in response to receiving the signal, activating a first actuator of the set of actuators to retract the cartridge while activating a second actuator of the set of actuators to advance the catheter such that the needle and the guidewire are retracted from the target vessel.

Variations of the present disclosure include a robotic system for inserting a needle, a catheter, and a guidewire into a body of a patient, using an electronic display, the robotic system including: a base; an arm linkage including a plurality of arm segments, where the plurality of arm segments includes a base arm coupled to the base and a device arm; a manipulation device coupled to the device arm, the manipulation device including an actuator assembly configured to advance one or more of the needle, the catheter, and the guidewire from a first portion of the manipulation device; a braking assembly configured to maintain the robotic system in a locked configuration that prevents movement of one or more of the plurality of arm segments and/or the manipulation device; an imaging device coupled to a first portion of the manipulation device, the imaging device configured to display a plurality of non-invasive images on the electronic display; a control interface positioned on a second portion of the manipulation device, the control interface including at least one input device and a first release switch; a second release switch spaced a distance from the second portion, wherein activation of the first release switch and the second release switch places the braking assembly in an unlocked configuration permitting repositioning of the arm linkage and/or the manipulation device; and a control unit operatively connected to the control interface, such that an input generated from the at least one input device causes the control unit to operate the actuator assembly.

Variations of the present disclosure include a robotic system for inserting a needle, a catheter, and a guidewire into a body of a patient, using an electronic display, the robotic system including: a base; an arm linkage including a plurality of arm segments, where the plurality of arm segments includes a base arm coupled to the base and a device arm; a manipulation device coupled to the device arm, the manipulation device including an actuator assembly configured to advance one or more of the needle, the catheter, and the guidewire from a first portion of the manipulation device; a braking assembly configured to maintain the robotic system in a locked configuration that prevents movements of one or more of the plurality of arm segments and/or the manipulation device; an imaging device coupled to a first portion of the manipulation device, the imaging device configured to display a plurality of non-invasive images on the electronic display; a control interface positioned on a second portion of the manipulation device, the control interface including at least one input device and a first release switch; and a control unit operatively connected to the control interface, such that an input generated from the at least one input device causes the control unit to operate the actuator assembly, wherein the control unit is configured to move the needle over a continuous path at a first displacement parameter and then a second displacement parameter.

Variations of the present disclosure include a robotic system for inserting a needle, a catheter, and a guidewire into a body of a patient, using an electronic display, the robotic system including: a base; an arm linkage including a plurality of arm segments, where the plurality of arm segments includes a base arm coupled to the base and a device arm; a manipulation device coupled to the device arm, the manipulation device including an actuator assembly configured to advance one or more of the needle, the catheter, and the guidewire from a first portion of the manipulation device; a braking assembly configured to maintain the robotic system in a locked configuration that prevents movements of one or more of the plurality of arm segments and/or the manipulation device; an imaging device coupled to a first portion of the manipulation device, the imaging device configured to display a plurality of non-invasive images on the electronic display; a control interface positioned on a second portion of the manipulation device, the control interface including a main input device and a first release switch; and a control unit operatively connected to the control interface, wherein the control unit is configured to selectively advance the needle, the catheter, the guidewire, or a combination thereof using the main input device to prevent an operator from having to disengage the main input device during operation of the robotic system.

Variations of the present disclosure include a robotic system for inserting a needle, a catheter, and a guidewire into a body of a patient, using an electronic display, the robotic system including: a base; an arm linkage including a plurality of arm segments, where the plurality of arm segments includes a base arm coupled to the base and a device arm; a manipulation device coupled to the device arm, the manipulation device including an actuator assembly configured to advance one or more of the needle, the catheter, and the guidewire from a first portion of the manipulation device; a braking assembly configured to maintain the robotic system in a locked configuration that prevents movements of one or more of the plurality of arm segments and/or the manipulation device; an imaging device coupled to a first portion of the manipulation device, the imaging device configured to simultaneously display one or more non-invasive images of the body on the electronic display, wherein the one or more non-invasive images include a plurality of transverse views along a transverse direction of the body and a plurality of longitudinal views along a longitudinal direction of the body; a control interface positioned on a second portion of the manipulation device, the control interface including at least one input device and a first release switch; and a control unit operatively connected to the control interface, wherein the control unit is configured to selectively advance the needle, the catheter, the guidewire, or a combination thereof using the at least one input device.

Variations of the present disclosure include a robotic system for inserting a needle, a catheter, and a guidewire into a body of a patient, using an electronic display, the robotic system including: a base; an arm linkage including a plurality of arm segments, where the plurality of arm segments includes a base arm coupled to the base and a device arm; a manipulation device coupled to the device arm, the manipulation device including an actuator assembly configured to advance one or more of the needle, the catheter, and the guidewire from a first portion of the manipulation device; a braking assembly configured to maintain the robotic system in a locked configuration that prevents movement of one or more of the plurality of arm segments and/or the manipulation device; an imaging device coupled to a first portion of the manipulation device, the imaging device configured to display a plurality of non-invasive images on the electronic display; a control interface positioned on a second portion of the manipulation device, the control interface including at least one input device and a first release switch; a second release switch spaced a distance from the second portion, wherein activation of the first release switch and the second release switch places the braking assembly in an unlocked configuration permitting repositioning of the arm linkage and/or the manipulation device; and a control unit operatively connected to the control interface, such that an input generated from the at least one input device causes the control unit to operate the actuator assembly.

Variations of the present disclosure include a robotic system for inserting a needle, a catheter, and a guidewire into a body of a patient, using an electronic display, the robotic system including: a base; an arm linkage including a plurality of arm segments, where the plurality of arm segments includes a base arm coupled to the base and a device arm; a manipulation device coupled to the device arm, the manipulation device including an actuator assembly configured to advance one or more of the needle, the catheter, and the guidewire from a first portion of the manipulation device; a braking assembly configured to maintain the robotic system in a locked configuration that prevents movements of one or more of the plurality of arm segments and/or the manipulation device; an imaging device coupled to a first portion of the manipulation device, the imaging device configured to display a plurality of non-invasive images on the electronic display; a control interface positioned on a second portion of the manipulation device, the control interface including at least one input device and a first release switch; and a control unit operatively connected to the control interface, such that an input generated from the at least one input device causes the control unit to operate the actuator assembly, wherein the control unit is configured to move the needle over a continuous path at a first displacement parameter and then a second displacement parameter.

Variations of the present disclosure include a robotic system for inserting a needle, a catheter, and a guidewire into a body of a patient, using an electronic display, the robotic system including: a base; an arm linkage including a plurality of arm segments, where the plurality of arm segments includes a base arm coupled to the base and a device arm; a manipulation device coupled to the device arm, the manipulation device including an actuator assembly configured to advance one or more of the needle, the catheter, and the guidewire from a first portion of the manipulation device; a braking assembly configured to maintain the robotic system in a locked configuration that prevents movements of one or more of the plurality of arm segments and/or the manipulation device; an imaging device coupled to a first portion of the manipulation device, the imaging device configured to display a plurality of non-invasive images on the electronic display; a control interface positioned on a second portion of the manipulation device, the control interface including a main input device and a first release switch; and a control unit operatively connected to the control interface, wherein the control unit is configured to selectively advance the needle, the catheter, the guidewire, or a combination thereof using the main input device to prevent an operator from having to disengage the main input device during operation of the robotic system.

Variations of the present disclosure include a robotic system for inserting a needle, a catheter, and a guidewire into a body of a patient, using an electronic display, the robotic system including: a base; an arm linkage including a plurality of arm segments, where the plurality of arm segments includes a base arm coupled to the base and a device arm; a manipulation device coupled to the device arm, the manipulation device including an actuator assembly configured to advance one or more of the needle, the catheter, and the guidewire from a first portion of the manipulation device; a braking assembly configured to maintain the robotic system in a locked configuration that prevents movements of one or more of the plurality of arm segments and/or the manipulation device; an imaging device coupled to a first portion of the manipulation device, the imaging device configured to simultaneously display one or more non-invasive images of the body on the electronic display, wherein the one or more non-invasive images include a plurality of transverse views along a transverse direction of the body and a plurality of longitudinal views along a longitudinal direction of the body; a control interface positioned on a second portion of the manipulation device, the control interface including at least one input device and a first release switch; and a control unit operatively connected to the control interface, wherein the control unit is configured to selectively advance the needle, the catheter, the guidewire, or a combination thereof using the at least one input device.

Other systems, processes, and features will become apparent to those skilled in the art upon examination of the following drawings and detailed description. It is intended that all such additional systems, processes, and features be included within this description, be within the scope of the present invention, and be protected by the accompanying claims.

### BRIEF DESCRIPTION OF THE FIGURES

The skilled artisan will understand that the drawings are primarily for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 is a block diagram that illustrates a system for facilitating vascular access, according to some embodiments.

FIG. 2 is a block diagram that illustrates a system for facilitating vascular access, according to some embodiments.

FIG. 3A is a block diagram that illustrates a robotic arm of a system for facilitating vascular access, according to some embodiments.

FIG. 3B is a block diagram that illustrates a robotic arm of a system for facilitating vascular access, according to some embodiments.

FIG. 4A is a block diagram that illustrates the base of a system for facilitating vascular access, according to some embodiments.

FIG. 4B is a block diagram that illustrates a control unit of the system of FIG. 4A, according to some embodiments.

FIG. 5 is a block diagram that illustrates components of a manipulation device and a cartridge assembly and interactions therebetween, according to some embodiments.

FIG. 6 is a flow diagram illustrating a method of performing a vascular access procedure, in accordance with some embodiments.

FIG. 7 is a flow diagram illustrating a method of using a vascular access system to perform the Seldinger technique, in accordance with some embodiments.

FIG. 8 is a flow diagram illustrating a method of using image data and/or other sensor data to perform a vascular access procedure, in accordance with some embodiments.

FIG. 9 is a flow diagram illustrating a method of retracting one or more interventional devices using a vascular access device, in accordance with some embodiments.

FIGS. 10A and 10B illustrate an example of a vascular access system.

FIGS. 10C and 10D illustrate examples of a patient support coupled to one or more support arms when moved away from the storage position shown in FIG. 10A.

FIG. 10E illustrates the vascular access system of FIG. 10A engaging a patient, in accordance with some embodiments.

FIG. 10F illustrates the vascular access system of FIG. 10A showing the attachment of a cartridge, in accordance with some embodiments.

FIG. 11A illustrates a robotic system in a first configuration for storage.

FIG. 11B illustrates the robotic system in a second configuration for vascular access.

FIGS. 12A-12B illustrate another example of a robotic system.

FIG. 13 illustrates a variation of a manipulation device and cartridge for use with a vascular access system.

FIG. 14 illustrates a transverse view and a longitudinal view of a blood vessel as captured by an ultrasound imaging device, in accordance with some embodiments.

FIG. 15A illustrates an example of the bottom or patient-contacting side of an imaging device as described herein.

FIG. 15B illustrates an imaging device and various views T1, T2, T3, L1, and L2, when the imaging device is located on a body part of a patient and generally aligned with a blood vessel as viewed from a side.

FIG. 16A shows an example of a user interface 150 that can be presented on any system display as described herein.

FIG. 16B shows a variation of the user interface that displays the various views T1, T2, T3, L1, and L2 from the imaging device.

FIG. 16C provides an illustration of another user interface screen where the target vessel is located in a region of the anatomy where T1, T2, and L1 provide the most relevant views to assist the caregiver in positioning the manipulation device over the target vessel.

FIG. 16D shows another feature of the system and user interface, where the user interface is configured to identify and display a virtual image of a vessel.

FIG. 16E is intended to illustrate the user interface when the needle path becomes aligned, or is substantially aligned, with a vessel in one or more views, T1, T2.

FIGS. 16F and 16G show another variation of a user interface to align a needle path with a vessel in one or more views.

FIG. 17A illustrates the user interface after the “position target vessel” phase and into the “advance needle phase”.

FIGS. 17B to 17D illustrate another feature of the system that relies on machine learning or other image processing to confirm the placement of the needle into a vessel.

FIGS. 17E and 17F represent additional steps of the catheterization procedure performed using the system.

FIG. 18A is a flow diagram illustrating a method for aligning multiple views of a blood vessel, in accordance with some embodiments.

FIG. 18B depicts a method for locking a position of a robotic arm, in accordance with some embodiments.

FIG. 19 illustrates a manipulation device with a ball joint, in accordance with some embodiments.

FIGS. 20A-20B illustrate a drape adaptor for an imaging device of a vascular access system, in accordance with some embodiments.

FIG. 21 illustrates a control system of a manipulation device of a vascular access system, in accordance with some embodiments.

FIG. 22 illustrates a method for retracting vascular access components, in accordance with some embodiments.

FIG. 23 is a block diagram that illustrates a control unit of a system for facilitating vascular access, in accordance with some embodiments.

FIG. 24 illustrates a method for automatically identifying and puncturing target tissue, in accordance with some embodiments.

FIG. 25 illustrates a method for puncturing tissue, in accordance with some embodiments.

### DETAILED DESCRIPTION

Robotic systems, devices, and methods for vascular access are described herein. In some embodiments, the robotic systems, devices, and methods described herein automate or semi-automate vascular access (e.g., the procedure of the Seldinger technique) in order to provide safe access to blood vessels (or vessels) and/or organ(s). The blood vessel(s) can be any suitable type of blood vessel(s), such as arteries (e.g., radial artery, femoral artery, etc.), veins (e.g., brachial vein, basilic vein, cephalic vein, femoral vein, internal jugular vein, median cubital vein, median antebrachial vein, etc.).

In some embodiments, the technology described herein includes a robotic system for facilitating vascular access. The robotic system can include a manipulation device coupled to a cartridge. The manipulation device and/or the cartridge can comprise or otherwise be attached to a guidewire, a needle, and a catheter that is to be positioned in a blood vessel of a subject. The robotic system and the manipulation device can be controlled by a user (e.g., an operator, a surgeon, etc.) using one or more input/output (I/O) devices. In some embodiments, the manipulation device can include an imaging device (e.g., an ultrasound array). The imaging device can provide the user with visual aid (e.g., ultrasound images of the blood vessel) of the procedure, such as the guidewire, the needle, and/or the catheter being inserted into a blood vessel. In some embodiments, the I/O device(s) can include a sensor (e.g., camera) that provides feedback (e.g., image data of the robotic system, manipulation device, and/or portion of a subject's body) to the robotic system as the manipulation device accesses a blood vessel. The robotic system can adjust the movement, position, and/or orientation of the guidewire, needle, and/or catheter based on data from the sensor so as to automate the procedure of vascular access. In some embodiments, the user can remotely control the robotic system and/or the manipulation device to perform the procedure based on the data from the sensor and the visual aid from the imaging device. In some embodiments, the robotic system can aid, guide, or augment a user manually operating the manipulation device. Further details of such a system are described below with reference to the figures.

### Vascular Access System and Components

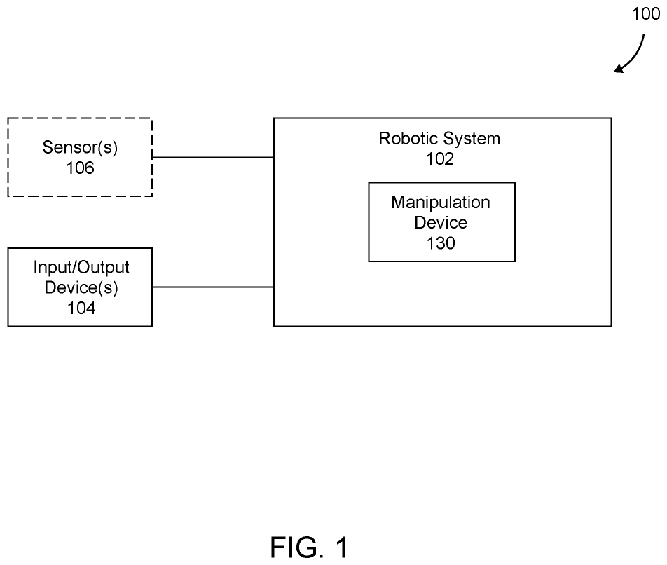

FIG. 1 is a high-level block diagram that illustrates a system 100, according to some embodiments. System 100 can be configured to automate and/or semi-automate a medical procedure for vascular access. System 100 includes a robotic system 102 including a manipulation device 130. The robotic system 102 and/or the manipulation device 130 can be communicably coupled to one or more I/O device(s) 104 (e.g., external and/or remote I/O devices). In some embodiments, the robotic system 102 and/or the manipulation device 130 can be optionally communicably coupled to one or more sensors 106 (e.g., external and/or remote sensors).

In some embodiments, the robotic system 102 can be any suitable robot. For instance, the robotic system 102 can include a robotic arm that can form a part of a robotic device. The robotic device itself can be an autonomous and/or semi-autonomous cart coupled to and/or integrated with the manipulation device. In some embodiments, the robotic device can include a base with a flat portion that is configured to support a patient on whom the medical procedure is to be performed, as further described herein. Alternatively, the robotic device can be an autonomous robot with humanoid features (e.g., arms, transport elements, head, base, etc.).

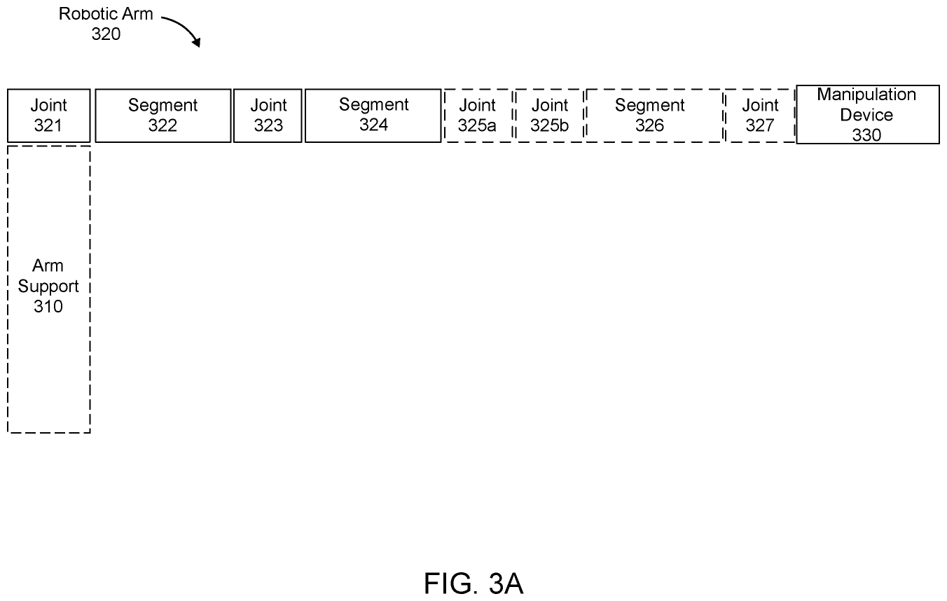

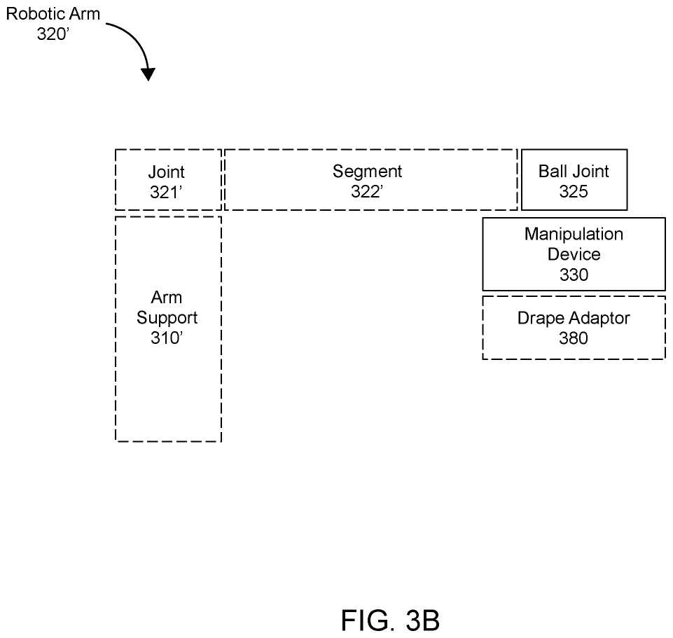

The robotic system 102 can include a robotic arm with two or more segments coupled together via joints, as further detailed with reference to FIGS. 3A-3B. In embodiments, the robotic arm can provide at least three, at least four, at least five, at least six, at least seven, at least eight, or at least nine degrees of freedom of movement, including values and sub-ranges therebetween. For example, the robotic arm can provide between three and six degrees of freedom. Each joint of the robotic arm can be configured to allow one or more degrees of freedom. For example, joints can provide for translation along and/or rotation of the robotic arm about one or more axes. In some embodiments, the joints can include locking mechanisms (e.g., brakes, etc.) configured to lock the position of the robotic arm. In some embodiments, one end segment of the robotic arm can include a coupling element. The coupling element can couple the robotic arm to the manipulation device 130. The other end segment of the robotic arm can be disposed on, affixed to, mounted on, and/or integrated with at least a portion of the robotic system 102. In some embodiments, the robotic arm can include one or more joints that can be configured to allow a portion of the robotic arm to move in a plurality of directions, such as, for example, a ball joint.

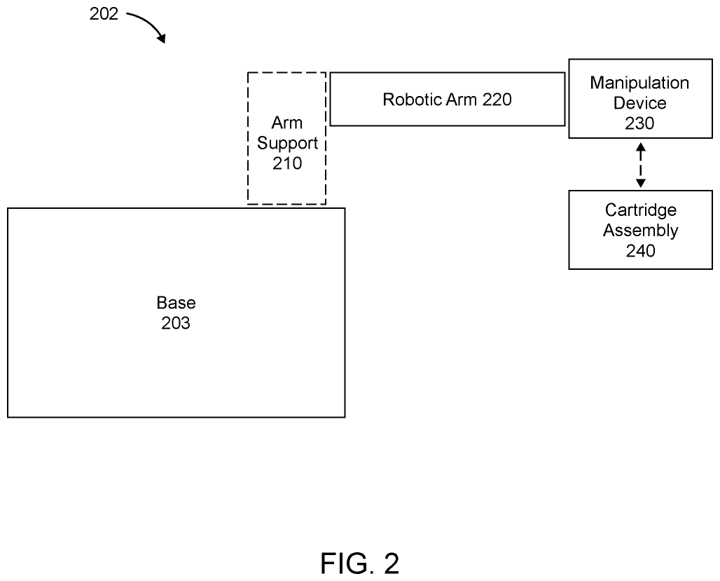

In some embodiments, the robotic arm can be disposed on, affixed to, mounted on, and/or integrated with a base (e.g., base of an autonomous and/or semi-autonomous cart) of the robotic system 102, as further detailed with reference to FIG. 2. In some embodiments, the base can carry the robotic arm, one or more I/O device(s) 104, and one or more sensor(s) 106. The base can be a movable base with one or more transport elements that can provide for translation along and/or rotation of the robotic system 102 along one or more axes. Additionally or alternatively, the base can be configured to be stationary. In some embodiments, the base can include a support surface configured to support patient anatomy (e.g., arm, leg, etc.) during a procedure. In some embodiments, the support surface can be selectively deployed or put away for storage. In some embodiments, the height of the support surface can be altered. In some embodiments, the base can include a locking mechanism to lock the movement of the transport elements and/or the movement of the base itself.

In some embodiments, the robotic system 102 can include a communication interface to enable communication with the I/O device(s) 104 and/or the sensor(s) 106. In some embodiments, the robotic system 102 can include a control unit to control the robotic system 102 (e.g., to control the base, robotic arm, etc.).

The robotic system 102 is described as a robotic arm disposed on, affixed to, mounted on, and/or integrated with a base solely for illustrative purposes. It should be readily understood that the robotic system 102 can be any suitable robotic component (e.g., robotic cart, humanoid robot, etc.) that can be coupled to one or more manipulation devices 130. For instance, the robotic system 102 can include multiple robotic arms that form a part of the robotic system 102. Each robotic arm can be coupled to a respective manipulation device. In such a scenario, the robotic system 102 may be configured to perform the medical procedure on multiple subjects substantially simultaneously. Additionally or alternatively, the robotic system 102 may include a robotic arm without a base. Additionally or alternatively, the robotic system 102 can be an autonomous humanoid robot (e.g., a robot with humanoid features such as a head, transport elements, manipulation elements, etc.) with a robotic arm for facilitating vascular access.

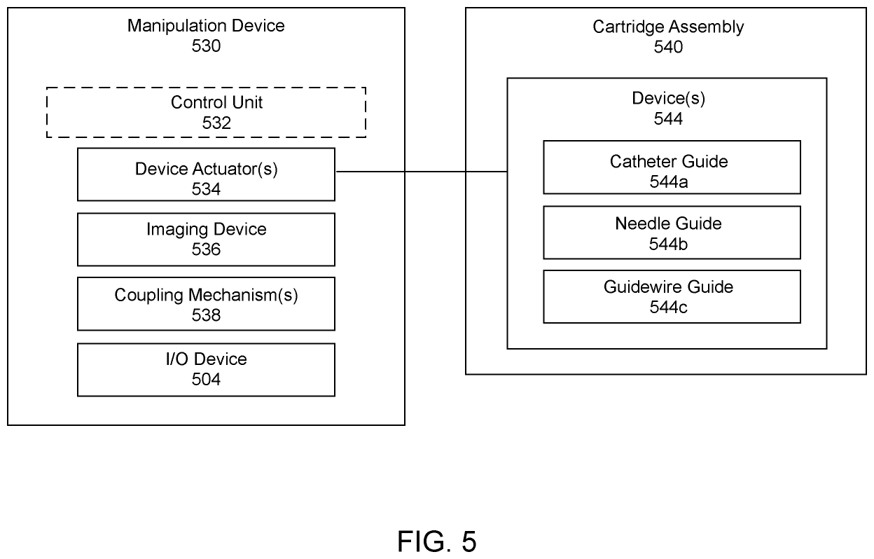

In some embodiments, the manipulation device 130 can be coupled to the robotic system 102 via a coupling element. The manipulation device 130 can be configured to drive movement of one or more components (e.g., a catheter, a needle, and/or a guidewire) to facilitate vascular access. The coupling element can include any type of mechanism that can couple the manipulation device 130 to the robotic system 102, such as, for example, a mechanical mechanism (e.g., a fastener, a latch, a mount, a joint), a magnetic mechanism, a friction fit, etc. The manipulation device 130 can be attached to a cartridge assembly (further described with reference to FIG. 5) that can include a needle, a catheter, and/or a guidewire to perform the medical procedure. In some embodiments, the manipulation device 130 can include one or more actuators that can actuate each of the needle, the catheter, and the guidewire. In some embodiments, one or more actuators are housed within the cartridge assembly. The actuators can enable the manipulation device 130 to perform the medical procedure. One or more actuators can be any suitable type of actuator. For instance, one or more actuators can include linear actuators with magnetic encoders.

In some embodiments, the robotic system 102 can include an imaging device (e.g., ultrasound array) to provide a user (e.g., an operator, a surgeon, etc.) with visual aid (e.g., ultrasound images showing one or more transverse views and/or one or more longitudinal views) as the medical procedure is performed (e.g., ultrasound images of the needle, the catheter, and/or the guidewire being inserted into a blood vessel of a subject). In some embodiments, the imaging device can be integrated and/or form part of the manipulation device, as further detailed with reference to FIG. 5.

The manipulation device 130 and/or the robotic system 102 can be communicably coupled to one or more I/O device(s) 104. An I/O device(s) 104 can be any suitable input device that can be configured to receive inputs from the user and/or any suitable output device that can be configured to send outputs to other devices and/or the user operating the robotic system 102. In some embodiments, the I/O device(s) 104 can be an integrated computing device that includes one or more components to both receive inputs and send outputs. Some non-limiting examples of integrated computing devices that can receive inputs from the user and send outputs to the user and/or to other devices, include computers (e.g., desktops, personal computers, laptops, etc.), tablets and e-readers (e.g., Apple iPad®, Samsung Galaxy® Tab, Microsoft Surface®, Amazon Kindle®, etc.), mobile devices and smartphones (e.g., Apple iPhone®, Samsung Galaxy®, Google Pixel®, etc.).

In some embodiments, the I/O device(s) 104 can be a user control, such as a joystick, a remote user control, keyboard, trackball, etc., that can receive input from the user. In some embodiments, the I/O device(s) 104 can be an audio device, such as a microphone and/or a speaker that receives audio input from the user. In such embodiments, the I/O device(s) 104 can additionally include a display device (e.g., a display, a touch screen, etc.) that displays output to the user. In some embodiments, the I/O device(s) 104 can be integrated and/or form part of the manipulation device, as further detailed with reference to FIG. 5 and as shown in FIG. 21.

The manipulation device 130 and/or the robotic system 102 can be optionally coupled to one or more sensor(s) 106. The sensor(s) 106 can be configured to capture image data of at least a part of the robotic system 102, the manipulation device 130, and/or at least a part of the subject as the robotic system 102 performs the medical procedure on the subject. The sensor(s) 106 can be an image sensor, such as a visual camera, stereo camera array, etc. The sensor(s) 106 can be operable to capture two-dimensional and/or three-dimensional images of the robotic system 102, the manipulation device 130, and/or the subject. In some embodiments, the sensor(s) 106 can be operated remotely by the user. For instance, the user can be in a location away from the system 100, and the sensor(s) 106 can be configured to be controlled remotely using one of the I/O device(s) 104. Alternatively, in some embodiments, the user can be in a location proximate to the system 100 and may not require any sensor(s) 106. In some embodiments, a user proximate to the system 100 can also operate and/or adjust one or more sensor(s) 106 of the system 100, e.g., one or more image sensors, to capture views of the environment for one or more remote users and/or for tracking/monitoring purposes.

In some embodiments, the sensor(s) 106 can be mounted on and/or can otherwise be an integral part of the I/O device(s) 104. For instance, the sensor(s) 106 can be attached to, coupled to, and/or otherwise be a part of the I/O device(s) 104. In some embodiments, the sensor(s) 106 can be mounted on the robotic system 102 itself. The sensor(s) 106 can be operable to move (e.g., rotational and/or translational motion) such that the sensor(s) 106 can capture image data from various angles. For instance, the sensor(s) 106 can be mounted on a pan/tilt mechanism to capture the image data. In some embodiments, the sensor(s) 106 can be a portable device, such as a handheld computer tablet, a smartphone with a camera, or a digital camera that is attached to, mounted on, and/or otherwise a part of the system 100.

In order to perform the medical procedure, the I/O device(s) 104 (e.g., user control such as a joystick, keyboard, remote control, trackball, etc.) can receive an input from the user. The input can be transmitted to the robotic system 102 and/or the manipulation device 130. For instance, the I/O device(s) 104 can receive an input to advance the needle, catheter, and/or guidewire into a blood vessel. As another example, the I/O device(s) 104 can receive an input associated with a locking mechanism (e.g., lock signal, unlock signal, etc.). The input can be transmitted from the I/O device(s) to the robotic system 102 via a communications interface. The robotic system 102 can cause the actuators in the manipulation device 130 to actuate the needle, catheter, and/or guidewire (e.g., included in a cartridge assembly) based on the input. The imaging device (e.g., ultrasound array) included in the manipulation device can provide a visual aid of the movement (e.g., the advancement) of the needle, catheter, and/or guidewire into the blood vessel. The visual aid (e.g., ultrasound images showing one or more transverse views and/or one or more longitudinal views) may be displayed on the I/O device(s) 104 (e.g., display device). In some embodiments, the I/O device(s) 104 can be configured to display a confirmation that at least a portion of the robotic system 102 is in a desired position. Subsequent input representing subsequent movement of the manipulation device 130 or one or more components in the manipulation device 130 (e.g., actuators actuating needle, catheter, and/or guidewire) can be provided to the I/O device(s) 104 based on the visual aid. For example, if the position of the needle, catheter, and/or guidewire in the blood vessel is incorrect, the visual aid (e.g., ultrasound images showing transverse view and/or longitudinal view) can guide the user to modify the input so that such component(s) advance to an appropriate location in the blood vessel.

In some embodiments, the sensor(s) 106 (e.g., camera) can provide image data of the robotic system 102, the manipulation device 130, and the subject to the user. The user can remotely control the manipulation device 130 based on the image data. For example, the image data may include images of the portion of the body of the subject that includes the blood vessel and the orientation and/or position of the manipulation device 130 with respect to the portion of the body. If the orientation and/or position of the manipulation device 130 with respect to the portion of the body is incorrect, the user can remotely control the manipulation device 130 (e.g., by sending instructions to the robotic system 102 via the I/O device(s) 104) so as to orient and/or position the manipulation device 130 as desired.

In some embodiments, the user can control the sensor(s) 106 remotely using the I/O device(s) 104. For instance, if the captured image data does not include images of the manipulation device 130 or the portion of the body, then the sensor(s) 106 can be remotely controlled by the user such that the angle of the sensor(s) 106 can be changed so as to capture the images of both the manipulation device 130 and the portion of the body. For example, the pan/tilt mechanism on which the sensor(s) are mounted can be remotely controlled by the I/O device(s) so as to capture the images as desired.

Subsequent inputs, such as to advance the needle, catheter, and/or guidewire, can be provided remotely through the I/O device(s) 104 based on the image data and the visual aid (e.g., ultrasound images) obtained from the imaging device (e.g., ultrasound array). In some embodiments, the robotic system 102 can be configured to automatically (e.g., via the control unit in the robotic system 102) adjust and/or lock the position and/or orientation of the manipulation device 130 or one or more components of the manipulation device 130 based on the image data and the visual aid. In this manner, the robotic system 102, along with the manipulation device 130, can perform the vascular access procedure (e.g., the Seldinger technique) in an automated and/or a semi-automated manner, such as with the user controlling the I/O device(s) 104 that in turn controls and actuates the robotic system 102 and/or the manipulation device 130.

### 1.0 Robotic System

FIG. 2 is a block diagram that illustrates a robotic system 202, similar to the system described herein, configured to provide a vascular access system, according to some embodiments. The robotic system 202 can include a base 203. The base 203 can be mechanically coupled to a robotic arm 220 via an arm support 210. The robotic arm 220 can be coupled to and/or integrated with a manipulation device 230. In some embodiments, a cartridge assembly 240 can be attached to the manipulation device 230. In some embodiments, the cartridge assembly 240 may be a cartridge configured to access a radial vein. In some embodiments, the cartridge assembly may be configured to access a jugular vein.

### 1.1 Robotic Arm

As depicted in FIG. 2, the base 203 can be coupled to the robotic arm 220. In some embodiments, the base 203 can be coupled directly to the robotic arm 220 and support the robotic arm 220 at a predefined distance above a ground surface or floor on which the base 203 is disposed. In some embodiments, an arm support 210 can be used to couple the base 203 to the robotic arm 220. The arm support 210 (similar to other arm supports described herein) can include a coupler, a stand, or other mounting device for coupling or attaching the robotic arm 220 to the base 203.

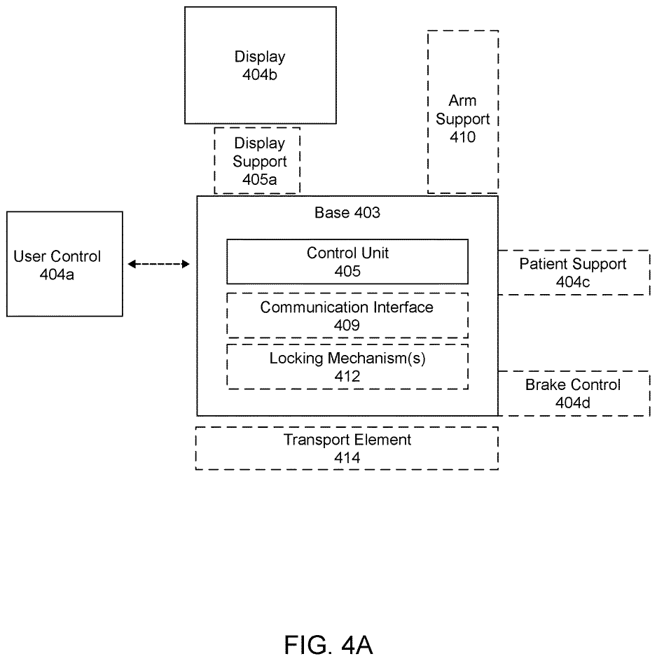

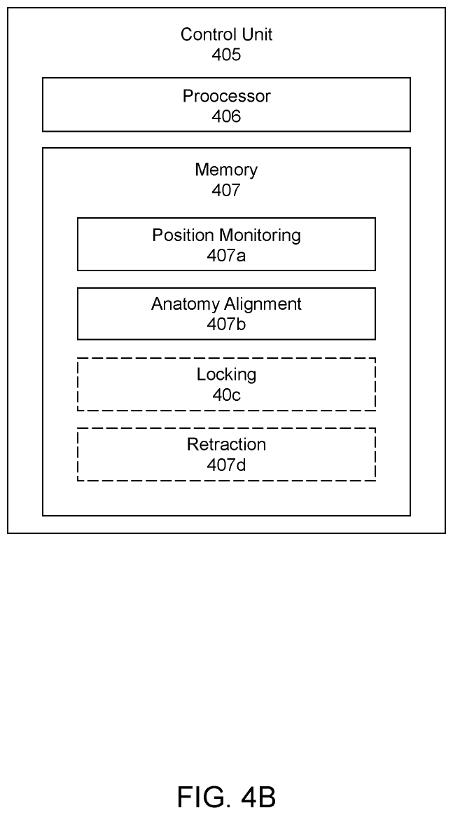

The robotic arm 220 can comprise two or more segments coupled together via joints. One end segment can be coupled to the arm support 210 and/or base 203. The other end segment can be integrated with and/or coupled to the manipulation device 230. In some embodiments, the robotic arm 220 can be actuated by one or more motors. In some embodiments, the robotic arm 220 can include one or more sensors to measure sensory information, including information relating to the robotic arm 220. Examples of sensors include position encoders, torque and/or force sensors, touch and/or tactile sensors, etc. The sensors can be disposed on or integrated with either the segments, the joints, or a combination of both. The sensory information can be transmitted to a control unit (e.g., control unit 405 in FIGS. 4A-4B) included in or attached to the base 203 and/or the manipulation device 230 (e.g., control unit 532 of FIG. 5). Additionally or alternatively, the sensory information can be transmitted to one or more I/O device(s) (e.g., I/O device(s) 104 in FIG. 1).

Referring generally to FIGS. 3A-3B, block diagrams that illustrate embodiments of a robotic arm 320, 320′ (e.g., structurally and/or functionally similar to robotic arm 220 in FIG. 2 and/or other robotic arms described herein) are shown, according to some embodiments.

FIG. 3A depicts a robotic arm 320 that can be implemented as an arm that includes two segments 322 and 324. Arm support 310 (e.g., similar to arm support 210 in FIG. 2) and segment 322 can be coupled together via joint 321. Segments 322 and 324 are coupled together via joint 323. In some embodiments, the robotic arm can optionally include segment 326. Segments 326 and 324 can be coupled together via joints 325a and 325b. In some embodiments, the manipulation device 330 (e.g., manipulation device 230 in FIG. 2) and segment 326 can be coupled together via joint 327. In other embodiments (e.g., embodiments that may not include segment 326), the manipulation device 330 and segment 324 can be coupled together via joint 325a. In yet other embodiments, the manipulation device 330 can be integrated with segment 324 or segment 326.

In some embodiments, the robotic arm 320 can have three proximal axes. A first proximal axis can be along the arm support 310 that enables vertical translation of the robotic arm 320 along the arm support 310. A second proximal axis can be along joint 321. A third proximal axis can be along joint 323. The three proximal axes can allow translation of the robotic arm 320 along the three-dimensional space. In some embodiments, the robotic arm can have three distal axes. A first distal axis can be along joint 325a, a second distal axis can be along joint 325b, and a third distal axis can be along joint 327. The three distal axes can allow rotation of the robotic arm 320 along the three-dimensional space (e.g., pitch, yaw, and roll). In this manner, the robotic arm can have six degrees of freedom. In some embodiments, the second proximal axis along joint 321, the third proximal axis along joint 323, and the first distal axis along 325a can comprise a planar Selective Compliance Articulating Robot Arm (SCARA) linkage. While three segments and five joints are depicted in FIG. 3A, one of ordinary skill in the art would understand that a robotic arm can include a different number of segments and/or joints.

In some embodiments, the robotic arm 320 can include a first joint (e.g., joint 321) which is configured to allow segment 322 (and segments distal thereto) to rotate about an axis of the first joint, a second joint (e.g., joint 323) which is configured to allow segment 324 (and segments distal thereto) to rotate about an axis of the second joint, and a third joint (e.g., joint 325a) which is configured to allow segment 326 (and segments distal thereto) to rotate about an axis of the third joint. In some embodiments, the axes of the first, second, and third joints can be orthogonal to each other. Therefore, the first, second, and third joints can define three degrees of freedom of movement of the robotic arm. In some embodiments, the robotic arm 320 can include an additional joint (e.g., joint 327), which can be configured to attach or couple to the manipulation device 330. The additional joint can enable the manipulation device to be rotated about at least one axis. In some embodiments, the additional joint can be implemented as a ball joint and enable the manipulation device to be rotated in multiple directions (e.g., 360-degree directional movement) relative to the robotic arm. The combination of joints and segments can enable the robotic arm 320 to be reconfigured (e.g., into different positions and/or orientations) to position the manipulation device 330 (and a cartridge coupled thereto) for insertion of a catheter, needle, and/or guidewire into the patient's vasculature.

In some embodiments, the robotic arm 320 can include locking mechanisms or devices for locking one or more components of the robotic arm 320. For example, the robotic arm can include one or more pulleys, magnets, etc. for locking one or more joints and/or a position of the robotic arm 320 relative to a base of a robotic system (e.g., base 203 of FIG. 2). The locking mechanisms can include, for example, mechanical locking devices (e.g., latches, ratchets, hydraulic locks), friction-based locking devices, electromagnetic locking devices, piezoelectric locking devices, etc. The locking mechanisms can be located at one or more joints, e.g., to lock a position of those joints and therefore the segments coupled thereto.

FIG. 3B is a block diagram that illustrates a robotic arm 320′ of a system for facilitating vascular access, in accordance with some embodiments. Unlike the robotic arm 320, the robotic arm 320′ includes a ball joint 325. The ball joint 325 is configured to couple the manipulation device 330 to the segment 322′ (e.g., a distalmost segment of the robotic arm). The ball joint 325 is configured to allow the manipulation device to swivel or rotate in multiple directions relative to a base. In some embodiments, the ball joint 325 includes a brake that, when activated, prevents further motion and/or rotation. In some embodiments, the brake may be a friction brake, a mechanical brake, an electric brake, and/or the like. In some embodiments, the brake may be activated in response to an erratic motion. In some embodiments, the brake may be activated based on the manipulation device 330 being placed in a desired position. In some embodiments, the operation of the brake of the ball joint 325 is controlled via a control unit (as described herein) associated with the base.

In some embodiments, the ball joint 325 is coupled to the base via a component configured to translate the ball joint. In some embodiments, the robotic arm 320′ includes one or more joint(s) 321′ and/or one or more segment(s) 324′ (as described herein). Optionally, the robotic arm 320′ can be coupled to the base via an arm support 310′. While FIG. 3B depicts a particular arrangement of the joints 321′ and segments 324′; one of ordinary skill in the art would understand that the robotic arm 320′ can include a different number of segments and/or joints. In some embodiments, the joint(s) 321′ can be configured as additional ball joints, similar functionally and/or structurally to the ball joint 325. Alternatively, one or more of the joint(s) 321′ can be rotational joints that allow segments distal to the joint to rotate about an axis of the joint. In an embodiment, the robotic arm 320′ can include a first joint 321′ that is configured to allow segments 324′ that are distal to the first joint 321′ to rotate about a first axis, a second joint 321′ that is configured to allow segments 324′ that are distal to the second joint 321′ to rotate about a second axis, and a third joint 321′ that is configured to allow segments 324′ to rotate about a third axis, where the first, second, and third axes are orthogonal to each other. The ball joint 325 can then be disposed on a distal end of the distalmost segment 324′, and be configured to couple the manipulation device 330 to that segment (and the rest of the robotic arm).

The arm support 310′, the joint(s) 321′, and/or the segment(s) 324′ can be included to position the robotic arm 320′ such that the manipulation device 330 is in a desirable position for an operation. Advantageously, having the ball joint 325 can enable the robotic arm 320′ to function with fewer segments and/or joints than the robotic arm 320. However, ball joints, such as the ball joint 325, may be more difficult to brake and/or lock compared to traditional single-axis rotational joints. Therefore, it can be desirable to have an effective mechanism for applying a brake to a ball joint of a robotic system to lock at least a portion of the robotic system.

Further examples of ball joints and mechanisms for braking them are described in U.S. Patent Application No. 63/560,288, entitled “VASCULAR ACCESS ROBOTIC SYSTEMS AND DEVICES INCLUDING CARTRIDGE ASSEMBLIES, AND METHODS THEREOF,” filed Mar. 1, 2024, which is incorporated herein by reference.

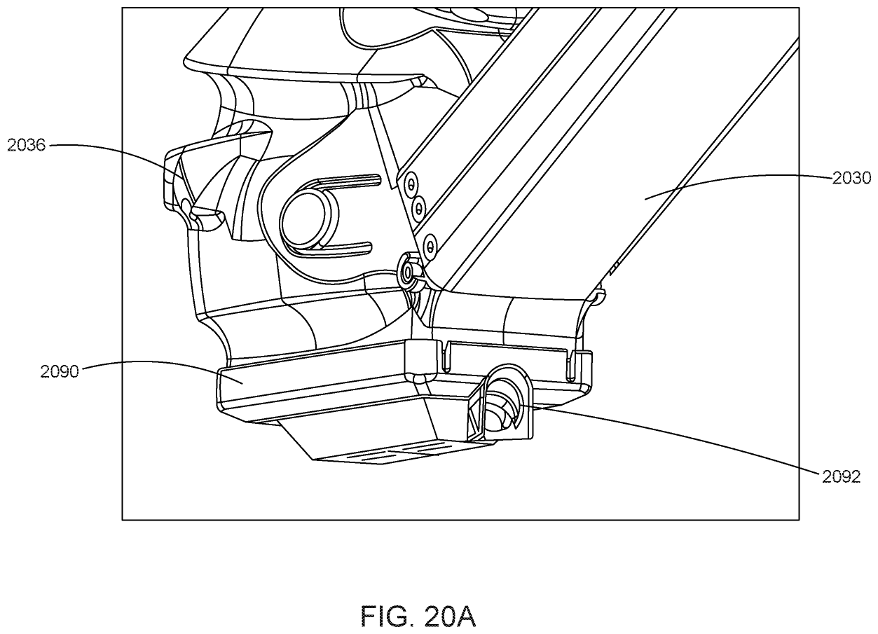

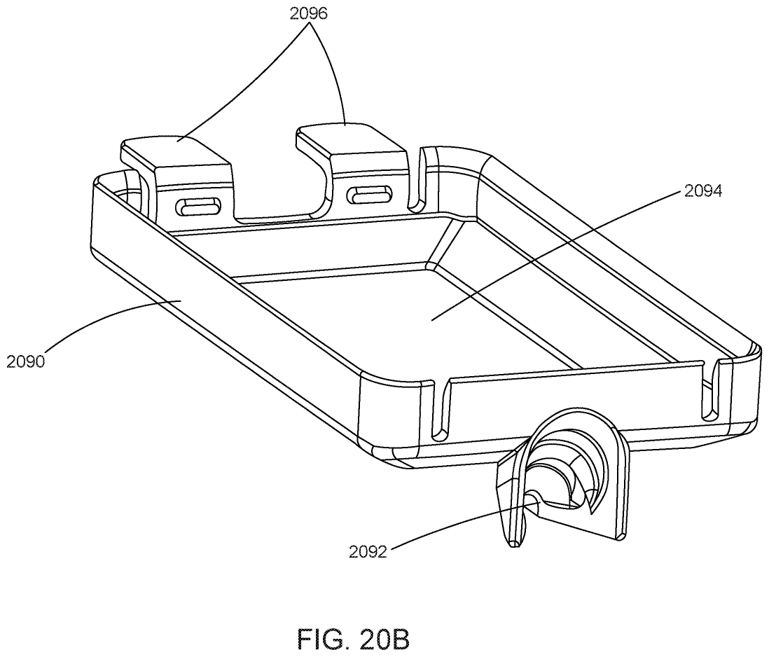

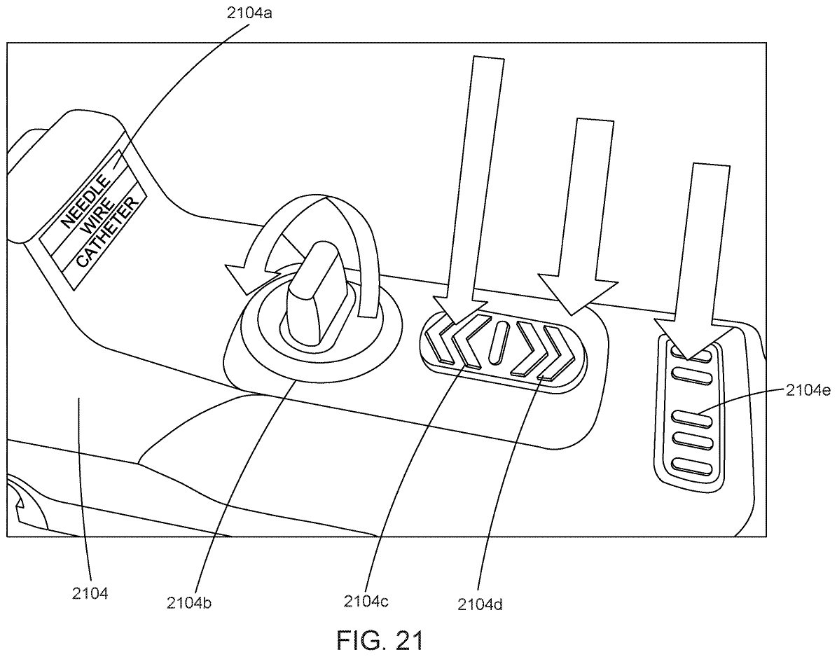

In some embodiments, the robotic arms 320, 320′ can include sensors in one or more components configured to measure the position, the acceleration, and/or the like of a portion of the robotic arms 320, 320′. In some embodiments, the robotic arms 320, 320′ (or any of the other robotic arms described herein) can be coupleable to a drape adaptor or sterile adaptor 380. In some embodiments, the drape adaptor 380 is configured to allow coupling between the manipulation device 330 and a cartridge (e.g., to drive movement of the catheter, guidewire, and/or needle) while providing a sterile barrier between the manipulation device 330 and the cartridge. Alternatively, or additionally, the drape adaptor 380 can be configured to fit around the imaging device of the robotic system (e.g., the transducer array) and to provide a sterile barrier between the imaging device and the patient. The drape adaptor 380 can be coupled or attached to a sterile drape, which can be configured to cover the robotic arms 320, 320′, the manipulation device 330, and/or other non-sterile components of the robotic system, e.g., to avoid contamination of the sterile field. In some embodiments, the drape adaptor 380 can be configured to detachably couple to the manipulation device 330. The drape adaptor 380 is further shown and described in reference to FIGS. 20A-20B.

### 1.2 Base

The base 203 can be any suitable base for positioning a manipulation device 230 of the vascular access system. For example, the base 203 can be a chassis supporting the robotic arm 220 and the manipulation device 230. In such scenarios, one or more electronic components, such as a control unit, a communications interface, etc., can be attached to and/or coupled to the base 203 (e.g., chassis). Alternatively, the base 203 can be a structure supporting the robotic arm 220 and the manipulation device 230 that houses one or more electronic components, such as a control unit, a communications interface, etc., within the base 203. Put differently, the outer structure of the base 203 can be a housing that encloses one or more electronic components. The robotic arm 220 and the manipulation device 230 can be supported on the outer structure. In some embodiments, the base 203 can be implemented as a surgical cart, which can support the robotic arm 220 and/or other components of the vascular access system. The cart can include transport elements (e.g., wheels, swivel casters, crawlers, tracks, etc.), which can be used to move the base from a first location to a second location near a patient. In some embodiments, the base 203 can be or include a surface with a flat portion configured to support a patient on whom the medical procedure is to be performed. For example, the base 203 can include a bed or other platform configured to support the patient. Additionally or alternatively, the base 203 can include a surface (e.g., an attachable extension such as an attachable arm rest or table) that can be configured to support at least a portion of the anatomy of the patient (e.g., leg, arm, etc.). A first portion of the robotic arm 220 can be coupled to the base 203 (e.g., bed, chassis, etc.). A second portion of the robotic arm 220 (e.g., a second portion opposite the first portion) can be coupled to the manipulation device 230. In some embodiments, the second portion of the robotic arm 220 coupled to the manipulation device 230 can be movable relative to the base 203 to position the needle, the guidewire, and the catheter for insertion into the target vessel of the patient.

FIG. 4A is a block diagram that illustrates a base 403 (e.g., structurally and/or functionally similar to base 203 in FIG. 2 and/or other bases described herein), according to some embodiments. Optionally, the bottom surface of the base 403 can include transport elements 414 that can provide for translation along and/or rotation of the robotic system (e.g., robotic system 202 in FIG. 2) along one or more axes. Transport elements 414 can be any suitable components configured for movement, such as, for example, a wheel, a swivel caster, a crawler, a track, etc. Transport elements 414 can enable the robotic system to move around.

For instance, the transport elements 414 can be swivel casters (e.g., 4 swivel casters coupled to 4 corners of the base 403) that provide three degrees of freedom to the robotic system. The swivel casters can allow for linear translations of the robotic system along two axes and rotation of the robotic system along one axis. These three degrees of freedom can enable a user (e.g., a surgeon and/or an operator) to achieve planar and rotational positioning of the base 403 and thereby planar and rotational positioning of the robotic system relative to a portion of a subject's body (e.g., arm, etc., on which the medical procedure is to be performed).

Optionally, the base 403 can include locking mechanism(s) or locking device(s) 412 to lock the movement of the base 403. For instance, once a user positions the robotic system 202 at an appropriate position (e.g., distance and/or height) with respect to the subject, the locking mechanism can be engaged to lock the position of the base 403 and the robotic system 202. The locking mechanism(s) 412 can lock the transport elements 414 (e.g., swivel casters), preventing the transport elements 414 from moving further. In some embodiments, the locking mechanism(s) 412 can automatically engage a lock. For instance, the locking mechanism(s) 412 can automatically lock the transport elements 414 as soon as the robotic system 202 is positioned at a desired location.

Optionally, the base 403 can include a communication interface 409. The communication interface 409 can be any suitable component that enables the base 403 and/or the robotic system 202 to communicate with I/O device(s) (e.g., I/O device(s) 104 in FIG. 1), sensor(s) (e.g., sensor(s) 106 in FIG. 1), or other suitable devices. In some embodiments, communication interface 409 can further enable the I/O device(s) to communicate with the transport elements 414 and/or locking mechanism(s) 412. In some embodiments, the I/O device(s) can include a user control 404a and/or a display 404b, as further detailed below.

As discussed above, the base 403 can support the robotic arm (e.g., robotic arm 220 in FIG. 2) and/or one or more I/O device(s) (e.g., I/O device(s) 104 in FIG. 1). For example, the base 403 can be coupled to the robotic arm via an arm support 410. One or more I/O device(s), such as, for example, user control 404a and display 404b, can be communicably coupled to the base 403. In some embodiments, the display 404b can additionally be mechanically coupled to the base 403 via the display support 405a. Display support 405a can be any suitable support that can attach and/or couple the display 404b to the base 403, such that the base 403 supports the display 404b. In some embodiments, display support 405a can enable adjustment(s) to be made to the position and/or orientation of the display 404b. Display 404b can be any suitable display device, such as a touch screen, a device displaying a graphical user interface, an audio device (e.g., microphone, speaker, etc.), a combination thereof, and/or the like.

In some embodiments, the user control 404a can additionally be attached to and/or integrated with the base 403. For instance, user control 404a can be integrated with the base 403 such that the base 403 supports the user control 404a. User control 404a can be any suitable device that can receive input from the user, such as a joystick, a remote user control, keyboard, trackball, etc. Additionally, or alternatively, a portion of the user control 404a can be implemented in a manipulation device (e.g., such as the manipulation device 230 of FIG. 2 and/or the manipulation device 330 of FIGS. 3A-3B).

Optionally, the base 403 can include a brake control 404d. The brake control 404d can be a foot brake, a pedal, a switch, a button, or other actuation device that is configured to activate one or more braking and/or locking mechanism(s) associated with the base 403, the robotic systems described herein, and/or the manipulation devices described herein. For example, the brake control 404d can be configured to be actuated by a user to activate one or more locking mechanisms 412 to lock a position of the base 403. As another example, the brake control 404d can be actuated to lock the position of the robotic arm and/or the manipulation device. In some embodiments, it may be desirable to actuate the brake control 404d when the base 403, the robotic arm, and/or the manipulation device are in a desired position, e.g., near a patient or target site. In some embodiments, it may be desirable to actuate the brake control 404d in response to an unexpected event or anomaly, e.g., to reduce the likelihood of injury to the patient or others. In some embodiments, the brake control 404d can be an emergency stop configured to stop the procedure (e.g., remove the needle, catheter, guidewire, etc.).

Optionally, the base 403 can include a patient support 404c. The patient support 404c may be a surface, platform, table, or the like configured to support the anatomy (or portion thereof) of a patient during the procedure. In some embodiments, the patient support 404c may be configured to support an arm, a leg, and/or the like. In some embodiments, the patient support 404c can be folded or stored (e.g., folded down vertically) for storage and/or transport. In some embodiments, the patient support 404c can be raised, extended, and/or otherwise positioned at a predetermined height and/or orientation for supporting the anatomy of a patient. In some embodiments, the patient support 404c can optionally include restraints (e.g., straps, etc.) and/or stabilizing portions (e.g., molded portions, raised portions, indentations, high-friction surfaces, etc.) configured to restrict the movement of at least a portion of the patient.

In some embodiments, the base 403 can include a control unit 405 to control and/or monitor one or more components of the robotic system (e.g., robotic system 202 in FIG. 2) such as the base 403, the robotic arm (e.g., robotic arm 220 in FIG. 2), the manipulation device (e.g., manipulation device 230 in FIG. 2), the cartridge assembly (e.g., cartridge assembly 240 in FIG. 2), and/or a combination thereof. The control unit 405 can include any suitable processing device (e.g., processor and/or processing circuitry) configured to run and/or execute functions associated with controlling and/or monitoring one or more components of the robotic system. In some embodiments, the control unit 405 may be communicatively coupled to or include a sensor (e.g., torque sensor, pressure sensors, ammeters, etc.) configured to monitor the state and/or operation of the robotic system.

FIG. 4B provides a more detailed view of functions, modules, or processes executed by the control unit 405. In an embodiment, the control unit 405 includes a processor 406 and a memory 407. The processor 406 can be a general-purpose processor, microcontroller, a Field Programmable Gate Array (FPGA), an Application Specific Integrated Circuit (ASIC), a Digital Signal Processor (DSP), and/or the like. The memory 407 can be, for example, a random access memory (RAM), a memory buffer, a hard drive, a flash memory, a database, an erasable programmable read-only memory (EPROM), an electrically erasable read-only memory (EEPROM), a read-only memory (ROM), and/or so forth. In some embodiments, the memory 407 stores instructions that cause the processor 406 to execute modules, processes, and/or functions associated with the robotic system. In some instances, the memory 407 can be operatively coupled to other control units (e.g., such as a control unit in a manipulation device).

As seen in FIG. 4B, the memory 407 can store processor-executable instructions that, when executed by a processor (e.g., processor 406), cause the processor to implement one or more functions, modules, or processes, such as position monitoring 407a and anatomy alignment 407b. Optionally, in some embodiments, the memory 407 can store instructions that cause the processor to implement locking 407c and/or retraction 407d. In some implementations, the memory 407 can include additional instructions for operating the control unit 405 and/or instructions for operating the robotic system.

Position monitoring 407a can include monitoring of one or more positions (e.g., special position, relative position, etc.) associated with the patient and/or the robotic system. For example, the one or more positions can include the position of at least a portion of the patient (e.g., target), the position of the robotic arm, the position of the manipulation device, the position of the base 403, and/or the like. In some embodiments, position monitoring 407a can include determining the position of the manipulation device relative to the target site of the patient. In some embodiments, position monitoring 407a can include using sensor data to determine the one or more positions. For example, the sensor data can include camera signals, force sensors (e.g., indicating the manipulation device has contacted the patient), motor encoders, and/or the like. In some embodiments, position monitoring 407a can include determining a location or state of one or more of the catheter, the guidewire, and/or the needle, e.g., as the catheter, guidewire, and needle are being inserted into a vessel (e.g., an artery). In some embodiments, position monitoring 407a can include monitoring a position or orientation of one or more of the catheter, the guidewire, and/or the needle relative to a skin surface of the patient. In some embodiments, position monitoring 407a may be automatic, semi-automatic, and/or manual. For example, a user can set positions using an input device. As another example, position monitoring 407a can be automatic based on the data.

In some embodiments, the position monitoring 407a can include determining if the cartridge and the manipulation device are coupled properly. For example, the position monitoring 407a can determine if a clutch mechanism between the cartridge and the manipulation device has been fully engaged. Furthermore, when the cartridge is coupled to the manipulation device, the position monitoring 407a can include monitoring the position of the needle, the guidewire, and/or the catheter from a zero position (e.g., a fully retracted position) associated with the needle, guidewire, and/or the catheter. For example, the processor 406 can be configured to initially zero the location of the catheter, the guidewire, and the needle by activating motor(s) to position the catheter, the guidewire, and the needle at a most proximal position. In other words, the processor 406 can be configured to activate motor(s) or actuator(s) of the system to set the position of the catheter, the guidewire, and the needle to a zero position (e.g., a predetermined position associated with a starting point of the procedure). The processor 406, while the catheter, the guidewire, and/or the needle are advanced (e.g., to insert the catheter, the guidewire, and/or the needle into a target vessel), can then monitor a position of the catheter, the guidewire, and/or the needle relative to its zero position. In some embodiments, the processor 406 can be configured to halt a procedure if the position it monitors deviates from one or more expected conditions or parameters. For example, if the processor 406 determines that the needle has extended too far (e.g., extended beyond an expected or predetermined distal position), then the processor 406 may implement locking 407c and/or retraction 407d, as further described below.

In some embodiments, position monitoring 407a can include monitoring a state, configuration, position, or orientation of one or more portions of the robotic arm (e.g., one or more segments and/or joints), and/or determining the state, position, and/or orientation of the manipulation device. For example, the processor can be configured to monitor (e.g., via information received from sensors) whether the robotic arm is operating normally. In some embodiments, the processor can be configured to determine whether a collision has occurred with respect to the robotic arm. In such embodiments, the processor can be configured to implement locking 407c and/or retraction 407d, e.g., to lock a movement of the robotic arm and/or retract one or more of the catheter, the guidewire, and/or the needle to avoid or reduce harm to the patient.

Anatomy alignment 407b can include receiving information from an imaging and/or sensing device for aligning the system with respect to a target vessel, or causing a display (e.g., a display associated with an imaging device) to present information to facilitate alignment. For example, the anatomy alignment 407b can include receiving information associated with aligning a trajectory of the catheter, the guidewire, and/or the needle with a target vessel. For example, the information can include ultrasound imaging signals corresponding to one or more views (e.g., a transverse view, a longitudinal view, etc.) of the target vessel. The anatomy alignment 407b can determine if the manipulation device has been positioned so that the trajectory of the catheter, the guidewire, and/or the needle is aligned with the target vessel, e.g., so that the manipulation device can safely advance the catheter, the guidewire, and/or the needle into the target vessel. For example, the anatomy alignment 407b can determine if the angle and/or location of the manipulation device is configured to allow the needle, the catheter, and/or the guidewire to be inserted into the target vessel safely (e.g., centrally, at an insertion angle that reduces damage, etc.).

In some embodiments, the anatomy alignment 407b includes receiving a plurality of views of the target vessel. In some embodiments, the anatomy alignment 407b can automatically determine if a trajectory of the catheter, the guidewire, and/or the needle is aligned with the target vessel based on the plurality of views. For example, in some embodiments, the anatomy alignment 407b may be configured to identify walls or boundaries of the target vessel in the images (e.g., using image processing, image segmentation, object recognition, etc.), and determine whether the trajectory of the catheter, the guidewire, and/or the needle is aligned with the target vessel. In some embodiments, the anatomy alignment 407b can be configured to generate a signal indicating if the catheter, the guidewire, and/or the needle is aligned with the target vessel. For example, if the catheter, the guidewire, and/or the needle are not aligned with the target vessel, the anatomy alignment 407b can include generating a first signal, e.g., a signal warning a user not to advance the needle. The user can then adjust a position of the manipulation device and cartridge coupled thereto (e.g., by moving or reconfiguring the robotic arm and/or angle of the manipulation device) to change a trajectory of the catheter, the guidewire, and/or the needle such that the catheter, the guidewire, and/or the needle is aligned with the target vessel. Alternatively, or additionally, the processor can cause the robotic system to automatically drive the movement of the robotic arm (e.g., via activating one or more motors of the robotic arm) to reposition the manipulation device and the cartridge to align the catheter, the guidewire, and/or the needle with the target vessel. Conversely, if the catheter, the guidewire, and/or the needle are aligned with the target vessel, the anatomy alignment 407b can include generating a second signal different from the first signal, e.g., a signal indicating that a user can advance the catheter, the guidewire, and/or the needle. In some embodiments, the first and second signals can include a sound, an output to be shown on the display 404b, a light pattern, a vibration, or other haptic feedback, and/or the like.

In some embodiments, the anatomy alignment 407b can be manually assisted, semi-automatic, or automatic. For example, in some embodiments, a user may provide an input identifying a target structure of the target vessel. The user may then manually align the trajectory of the catheter, the guidewire, and/or the needle with the target vessel using a visual aid to center the target structure for needle insertion. In some embodiments, such as when there are multiple views in the visual aid, the user may align the trajectory with centers or centroids of the target structure at multiple points (e.g., multiple transverse views). It can be beneficial to view multiple transverse views such that a user can confirm whether the trajectory of the catheter, the guidewire, and/or the needle is aligned along the length of the target vessel. This can avoid the catheter, the guidewire, and/or the needle being aligned with a first portion of the target vessel while not being aligned with a second portion of the target vessel.

In some embodiments, the anatomy alignment 407b can include implementing one or more algorithms, machine learning models, and/or the like, e.g., for identifying the target structure and/or determining whether the trajectory of the catheter, the guidewire, and/or the needle is aligned with the target structure. In some embodiments, a computer vision model can be used for monitoring sensor output associated with a blood vessel (and/or an area around the blood vessel) of a patient. In some embodiments, structure recognition methods can be used for automatically identifying and/or detecting the target structure based on sensor data. For example, the structure recognition methods can include grayscale threshold analysis, edge detection algorithms, machine learning-based structure recognition, convolutional neural networks, real-time feature extraction, pattern matching, Doppler flow detection, cross-sectional area analysis, adaptive region growing, contour analysis, image segmentation, and/or the like.

After the target structure has been identified in one or more views of the target vessel (e.g., one or more transverse views), the anatomy alignment 407b can include determining a centroid (e.g., center point) of the target structure. In some embodiments, the centroid is associated with a desired insertion position for the catheter, the guidewire, and/or the needle. In some embodiments, the user can manually align the manipulation device with the target structure, e.g., manually move or reconfigure the robotic arm and/or position or orientation of the manipulation device to position the catheter, the guidewire, and/or the needle in alignment with the target vessel. In some embodiments, the anatomy alignment 407b can generate feedback based on the alignment of the catheter, the guidewire, and/or the needle with the target vessel. For example, the feedback can include instructions for directing a user to move the manipulation device so that the catheter, the guidewire, and/or the needle are aligned with the target vessel. In some embodiments, based on the location of the centroid and an expected trajectory of the catheter, the guidewire, and/or the needle, the anatomy alignment 407b can include automatically operating the robotic system (e.g., activating one or more motors of the robotic arm) to move the manipulation device (and cartridge coupled thereto) into a desired position that aligns the catheter, the guidewire, and/or the needle with the target vessel.

Locking 407c can include monitoring for one or more locking conditions, operating one or more locking mechanisms of the robotic system when a locking condition is present, and/or unlocking the one or more locking mechanisms after a predetermined period of time or after a locking condition has been resolved. For example, a locking condition can include a situation where it may be desirable for the position of one or more of the components of the robotic system to be locked. For example, a locking condition can include when the robotic arm, manipulation device, or other components of the robotic system are in position for performing a vascular access procedure, when there is a fault condition (e.g., a collision, unexpected movement, etc.), and/or as indicated by a user. In some embodiments, locking 407c can be manual, semi-automatic, and/or automatic. For example, user inputs can indicate that locking is desired. Specifically, the user inputs can be input via one or more of the user control 404a, the brake control 404d, the manipulation device, via a touchscreen such as the display 404b, and/or the like.

In some embodiments, locking 407c can include locking the transport element 414 when the base 403 is positioned in a desired location (e.g., a predetermined distance from a patient or a patient bed). In some embodiments, the locking 407c can include locking or terminating the movement, advancement, and/or retraction of the needle, catheter, and/or guidewire. In some embodiments, the locking 407c can include locking the advancement of the needle, e.g., when the needle trajectory is not aligned with the target vessel. For example, if the needle trajectory is not aligned with the centroid(s) of the target vessel, the needle can be locked from advancing into the target vessel, e.g., to prevent or decrease the likelihood of undesired vessel puncturing. In some embodiments, locking 407c can include locking guidewire retraction during a guidewire advance phase, locking catheter retraction during a catheter advance phase, and/or other locking of the movement of the catheter, the guidewire, and/or the needle.

In some embodiments, the locking 407c can include locking the position and/or configuration of the robotic arm. For example, when the position of the manipulation device provides for alignment between the catheter, the guidewire, and/or the needle and the target vessel (e.g., during anatomy alignment 407b), the position and/or configuration of the robotic arm and/or manipulation device can be locked. Specifically, once the manipulation device has been positioned to align the trajectory of the catheter, the guidewire, and/or the needle with the centroid(s) of the target vessel, the locking 407c can include locking the robotic arm in the desired position so that the manipulation device remains in the desired position during the procedure. In some embodiments, the locking 407c can include monitoring one or more sensors to actuate the locking mechanism. For example, the one or more sensors can include an accelerometer configured to determine if one or more components of the robotic system fall outside of an expected range of motion. For example, a spike in an accelerometer reading can indicate that the system has been unintentionally moved, bumped, collided with, and/or the like. To prevent damage associated with this spike, a locking mechanism can be actuated to maintain a position of one or more components of the robotic system, e.g., to reduce the likelihood of inadvertent injury to the patient. In some embodiments, the locking 407c can include unlocking the locking mechanism(s). In some embodiments, the locking mechanism(s) can be unlocked based on a user signal, after a predetermined amount of time, based on a sensor signal, and/or the like.

Retraction 407d may include retracting, by the manipulation device (e.g., motors and/or linear actuators), the needle, the catheter, and/or the guidewire. In some embodiments, the needle, catheter, and/or the guidewire can be retracted based on a procedure completion signal, a user input, a sensor signal, and/or the like. For example, after a catheter has been inserted into the target vessel successfully, retraction of the needle and/or the guidewire may be manually and/or automatically started. As another example, a user can indicate that retraction of the needle, catheter, and/or the guidewire is desired based on determining that the procedure is complete or if a problem is occurring. In some embodiments, retraction 407d can include retracting the needle, catheter, and/or the guidewire based on a signal indicating an anomaly, such as a spike in acceleration, as discussed in more detail above.

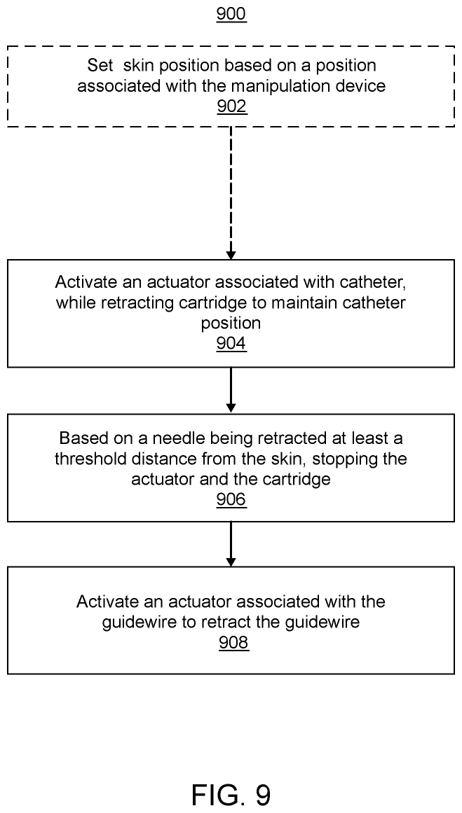

In some embodiments, retraction 407d can include retracting the needle, catheter, and/or the guidewire in a predetermined order. In some embodiments, the predetermined order can be configured to reduce strain on the vessel and/or reduce inadvertent injury. For example, if the catheter has been inserted successfully, retraction 407d can include retracting the needle and the guidewire. Specifically, a first actuator for retracting the needle and the guidewire can be activated (e.g., a motor that is configured to move the entire cartridge or a carriage supporting the cartridge proximally) while a second actuator associated with advancing the catheter in the opposite direction can be activated, e.g., so as to retract the needle and the guidewire while maintaining the position of the catheter in the vessel. In some embodiments, the needle is retracted to a predetermined height above the skin of the patient to prevent or decrease the likelihood of a puncture. After the needle has been retracted, the guidewire is retracted, leaving only the catheter in the vessel. In some embodiments, retraction 407d can include retracting the catheter, the guidewire, and the needle, e.g., if the control unit 405 receives a signal indicating an all-out signal (e.g., needle, catheter, and the guidewire are to be retracted). In such embodiments, retraction 407d can include first retracting the needle (e.g., to a predetermined height) and then retracting the catheter and/or the wire. Retraction 407d is described in more detail in reference to FIG. 9.

### 2.0 Manipulation Device and Cartridge Assembly

Referring back to FIG. 2, an end segment of the robotic arm 220 can be coupled to the manipulation device 230. The manipulation device 230 can be attached to a cartridge assembly 240. Further details of the components of an example manipulation device and an example cartridge assembly are described below.

FIG. 5 is a block diagram that illustrates a manipulation device 530 (e.g., similar to manipulation device 230 in FIG. 2 and/or other manipulation devices described herein) and a cartridge assembly 540 (e.g., similar to cartridge assembly in FIG. 2 and/or other cartridge assemblies described herein), according to some embodiments. In some embodiments, the manipulation device 530 can include a coupling mechanism 538, an imaging device 536, one or more device actuator(s) 534 (e.g., a catheter actuator, a guidewire actuator, a needle actuator, and/or an entire cartridge actuator), and optionally a control unit 532. In some embodiments, one or more of the device actuator(s) 534 (or a portion thereof) may be housed within the cartridge assembly 540. For example, the manipulation device 530 can include a plurality of motors that drive the movement of one or more linear actuators, where at least a portion of a linear actuator is located within the cartridge assembly 540. Any suitable permutation of the device actuator(s) 534 in the manipulation device 530 and/or the cartridge assembly 540 can be possible.

In some embodiments, an actuator that is configured to actuate the needle (e.g., a needle actuator) can be configured to actuate other components and/or devices (e.g., the catheter and/or the guidewire). In an embodiment, a first actuator can be configured to move the catheter, the needle, and the guidewire (or to move the entire cartridge assembly 540), a second actuator can be configured to move the guidewire relative to the needle and the catheter, and a third actuator can be configured to move the catheter relative to the needle and the guidewire.

In some embodiments, the device actuator(s) 534 can include one or more linear actuators. Each linear actuator can include a motor, a screw shaft, and a ball screw or other follower. Alternatively, each linear actuator can include a hydraulic actuator. In some embodiments, the motors that drive the movement of the screw shaft or other components of the linear actuators can be disposed in the manipulation device 530, while other components of the linear actuators (e.g., screw shaft, ball screw) can be disposed in the cartridge assembly 540.

In some embodiments, the cartridge assembly 540 can include the device(s) 544 (e.g., interventional devices) such as a catheter, a needle, and/or a guidewire. Alternatively, the manipulation device 530 can include some of the device(s) 544 while the cartridge assembly 540 can include other device(s) 544. For instance, the manipulation device 530 can include a catheter and a guidewire, while the cartridge assembly 540 can include the needle. Similarly, the manipulation device 530 can include the guidewire and the needle, while the cartridge assembly 540 can include the catheter. In a similar manner, any suitable permutation of the catheter, the needle, and the guidewire in the manipulation device 530 and/or the cartridge assembly 540 can be possible. In some embodiments, the guidewire, the needle, and the catheter can be arranged coaxially. For example, the guidewire can be disposed within a lumen of the needle, and the needle can be disposed within a lumen of the catheter. In some embodiments, a length of the catheter can be about 40 mm. In some embodiments, a length of the needle can be a little more than 40 mm (40 mm plus bevel length) such that the needle can extend past the catheter. In some embodiments, the guidewire can be 142 mm long such that at least 50 mm of the guidewire can extend past the needle tip. In some embodiments, the cartridge assembly 540 can be configured to store the guidewire in a linear state.

The imaging device 536 in the manipulation device 530 can provide the user with a visual aid of a blood vessel as the medical procedure is being performed. For example, the imaging device can be any suitable imaging device that can capture a visual representation of the blood vessel. Some non-limiting examples of the imaging device 536 can include ultrasound imaging devices, fluoroscopes, cameras, etc.

In some embodiments, the imaging device 536 can be an ultrasound array located on the manipulation device 230. The ultrasound array can provide two-dimensional ultrasound images along one or more longitudinal planes and/or transverse planes. The ultrasound images with the transverse view of a blood vessel can show the radial cross-section of the blood vessel, and the ultrasound images with the longitudinal view of the blood vessel can show the axial cross-section of the blood vessel. In some embodiments, the imaging device 536 can include an ultrasound array configured to provide a plurality of transverse and/or longitudinal views. Including a plurality of views can aid in alignment of the manipulation device 530 (and therefore the trajectory of the catheter, the guidewire, and/or the needle) with the blood vessel. In some embodiments, the imaging device 536 can be configured to obtain three-dimensional ultrasound images of the blood vessel.

The manipulation device 530 and the cartridge assembly 540 can each include a portion of one or more device actuator(s) 534. The device actuator(s) 534 can be configured to actuate the needle, the catheter, and/or the guidewire. For example, the manipulation device 530 can include linear actuators to actuate the device(s) 544. The linear actuators can include a ball screw shaft supported by ball screw bearings. A motor can be coupled to each device actuator 534 to drive the movement of a ball screw nut along the shaft. A magnetic encoder coupled to the motor can sinusoidally commutate the motor. A linear circulating ball bearing can be coupled to the ball screw nut that is fixed on the ball screw shaft. For instance, the linear circulating ball bearing can be coupled to the ball screw nut on the ball screw shaft via a carriage block. As the ball screw shaft rotates (e.g., owing to the rotation of the motor's rotor), the ball screw nut translates as it is constrained by the linear circulating ball bearing through the carriage block. The translation of the ball screw nut can, in turn, actuate a device(s) 544 along a linear axis. Accordingly, each of the needle, catheter, and guidewire can be actuated along a linear axis by a respective linear actuator. In some embodiments, a portion of the one or more device actuator(s) 534 is located in the manipulation device 530, and a corresponding portion of the one or more device actuator(s) 534 is located in the cartridge assembly 540. For example, a motor of a linear actuator may be located in the manipulation device 530, and a corresponding ball screw may be located in the cartridge assembly 540 and operably coupled to the motor.

In some embodiments, each of the needle, catheter, and guidewire can be attached to a respective guide that guides the device(s) 544 along the linear axis as the device(s) are being actuated by the linear actuators (e.g., device actuator(s) 534 included in manipulation device 530). In some embodiments, the guides can be included in the cartridge assembly 540 and can be attached to the respective device(s) 544. For example, a needle guide 544b included in the cartridge assembly 540 can be attached to the needle, a catheter guide 544a included in the cartridge assembly 540 can be attached to the catheter, and a guidewire guide 544c included in the cartridge assembly 540 can be attached to the guidewire. In some embodiments, the catheter guide 544a, the needle guide 544b, and the guidewire guide 544c can each include a coupling element that can couple with the coupling mechanism 538 in the manipulation device 530. Alternatively, in some embodiments, one or more of the needle, the catheter, or the guidewire may not include a guide. For example, the needle may not include a guide but can be coupled to move with the cartridge assembly 540 when the entire cartridge assembly 540 moves.

The manipulation device 530 can include a coupling mechanism 538 that couples the cartridge assembly 540 (e.g., the coupling element in the cartridge assembly 540) to the manipulation device 530, such as, for example, a mechanical mechanism (e.g., a fastener, a latch, a mount, a platform, a plate, a clip, etc.), a magnetic mechanism, a friction fit, etc. In some embodiments, the manipulation device 530 and the cartridge assembly 540 are operably coupled to allow for at least a portion of the device actuator(s) 534 in the manipulation device 530 to interface with corresponding portions of device actuator(s) 534 in the cartridge assembly. For example, the manipulation device 530 may be coupled to the cartridge assembly 540 such that motors in the manipulation device 530 may operate ball screws in the cartridge assembly 540, which may operate device(s) 544. In some embodiments, the cartridge assembly 540 is coupled to the manipulation device 530 such that the device actuator(s) 534 in the manipulation device 530 may translate the cartridge assembly along the manipulation device 530.

In some embodiments, the coupling mechanism 538 can be a mechanical mechanism. For example, the coupling mechanism 538 can include a cavity such that the catheter guide 544a, the needle guide 544b, and the guidewire guide 544c included in the cartridge assembly 540 can fit within the cavity. Alternatively, in some embodiments, one or more of the needle, the catheter, or the guidewire may not include a guide. For example, the needle may not include a guide but can be coupled to move with the cartridge assembly 540 when the entire cartridge assembly 540 moves. In some embodiments, the coupling mechanism 538 can be a combination of the magnetic mechanism and the mechanical mechanism.

In some embodiments, the coupling mechanism 538 includes an adaptor for facilitating coupling via the manipulation device 530 and the cartridge assembly 540. The adaptor may be configured to allow for the manipulation device 530 to be isolated (e.g., from potential contaminants, etc.) during operation. For example, the coupling mechanism 538 can be or form part of a sterile interface or adaptor, e.g., allowing for the cartridge assembly 540 to couple to the manipulation device 530 without compromising a sterile field. In particular, the cartridge assembly 540 can be a sterile component prior to use, while the manipulation device 530, the robotic arm, and/or other portions of the robotic system may not be sterile. Therefore, a sterile adaptor (e.g., including a platform, latches, capstans, or other structure) can be configured to allow components of the cartridge assembly 540 (e.g., catheter, needle, guidewire, guide(s), and/or actuator components (e.g., screw shaft(s))) to be operatively coupled to components of the manipulation device 530 (e.g., motors, etc.). The sterile adaptor can be attached to a sterile drape, which can be draped over the robotic arm, the manipulation device 530, and/or other components of the robotic system in use. In some embodiments, the coupling mechanism 538 may include a clutch mechanism between the cartridge assembly 540 and the manipulation device 530 that provides mechanical coupling while allowing for the manipulation device 530 to be isolated.

The manipulation device 530 includes an I/O device 504 configured to allow the user to control one or more operations during a vascular access procedure. For example, the user can control which of the needle, guidewire, and/or the catheter is being inserted, as well as the speed of insertion. In some embodiments, the I/O device 504 can be implemented into a handle of the manipulation device, as shown in FIG. 21.

In some embodiments, the manipulation device 530 can optionally include a control unit 532 to control the actuation of the device actuator(s) 534. Control unit 532 can be any suitable processing device configured to run and/or execute functions associated with controlling the device actuator(s) 534. Control unit 532 can include any suitable processor(s) that can be configured to execute modules, functions, and/or processes. In some embodiments, the processor(s) can be a general-purpose processor, a Field Programmable Gate Array (FPGA), an Application Specific Integrated Circuit (ASIC), a Digital Signal Processor (DSP), and/or the like.

In some embodiments, the control unit 532 can be configured to determine one or more limits associated with the cartridge assembly 540. For example, the one or more limits can include determining a needle, guidewire, and/or catheter range of travel based on the type of cartridge coupled to the manipulation device. In some embodiments, the control unit 532 can determine the type of cartridge based on a radio-frequency identification, a near-field communication, a QR code, a user input, and/or the like.

Further examples of manipulation devices and cartridge assemblies are described in U.S. patent application Ser. No. 18/587,711, entitled “ROBOTIC SYSTEMS, DEVICES, AND METHODS FOR VASCULAR ACCESS,” filed Feb. 26, 2024, and/or in PCT Patent Application No. PCT/US2023/085933, entitled “VASCULAR ACCESS ROBOTIC SYSTEMS AND DEVICES INCLUDING CARTRIDGE ASSEMBLIES, AND METHODS THEREOF,” filed Dec. 26, 2023, both of which are incorporated herein by reference.

### Methods

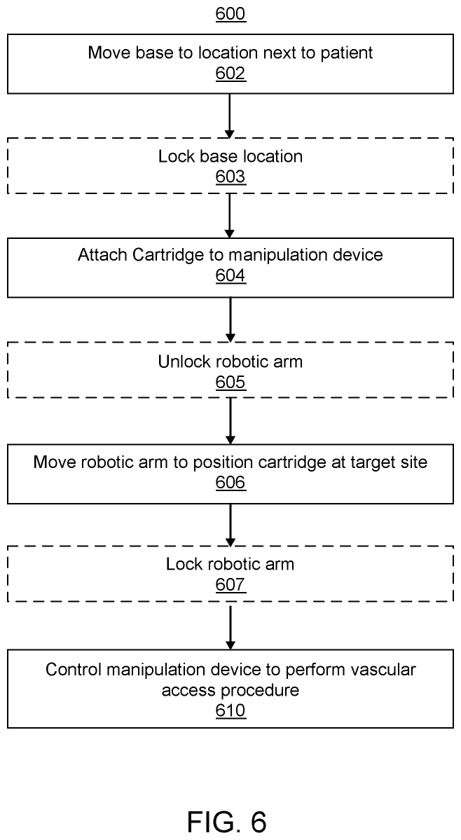

FIG. 6 is a flow diagram illustrating a method 600 of performing a medical procedure (e.g., using system 100 in FIG. 1), in accordance with some embodiments. A robotic system, such as, for example, robotic system 102 in FIG. 1 and/or any of the other robotic systems described herein, can perform the medical procedure (e.g., vascular access procedure) in an automated and/or semi-automated manner. At 602, a base (e.g., base 203 in FIG. 2 or base 403 in FIG. 4A) can be moved to a location next to a subject (e.g., a patient). As discussed above, the base can include transport elements (e.g., swivel casters) that can allow for translational and rotational movement of the robotic system. The base can be moved to a suitable position from the subject, such that the medical procedure can be performed on the subject. For example, the base can be moved to a position that allows a robotic arm (e.g., robotic arm 220 in FIG. 2 or robotic arm 320 in FIG. 3A) to access a portion of the subject's body (e.g., the patient's arm on which the medical procedure is to be performed).

In some embodiments, the base can optionally be raised to position the robotic arm such that at least a portion of the manipulation device and/or the cartridge assembly touches the skin of the subject. In some embodiments, an I/O device (e.g., I/O device(s) 104 in FIG. 1 or user control 404a in FIG. 4A) communicably coupled to the base and/or communicably coupled to the transport elements included in the base can transmit instructions to move the base. For example, the I/O device can be configured to transmit instructions to an actuator controlling the movements of the swivel caster. In other embodiments, the base can be moved manually by a user.

In some embodiments, a portion of the base and/or a patient support that is coupleable to the base (e.g., the patient support 404c of FIG. 1) can be configured to support target anatomy of a patient. The patient support can be deployed when the base is moved to the target location next to the patient. In some embodiments, the height of the patient support can be adjusted to a desired height that allows for desired access to the anatomy and/or patient comfort. Once the base and/or patient support is moved to a suitable location near the subject (e.g., suitable distance and/or suitable height), the base can be locked (e.g., using locking mechanism(s) 412 in FIG. 4A) to lock the location of the base, at 603 in FIG. 6.

At 604, a cartridge assembly (e.g., cartridge assembly 240 in FIG. 2 or cartridge assembly 540 in FIG. 5) can be attached to a manipulation device (e.g., manipulation device 130 in FIG. 1, manipulation device 230 in FIG. 2, manipulation device 330 in FIGS. 3A-3B, or manipulation device 530 in FIG. 5). The manipulation device can be coupled to or otherwise be a part of the robotic arm. In some embodiments, the manipulation device can include coupling mechanism(s) (e.g., coupling mechanism(s) 538 in FIG. 5) to couple the cartridge assembly to the manipulation device. For example, the manipulation device can include a permanent electromagnet that magnetically couples the manipulation device to the cartridge assembly. A coupling element (e.g., puck) in the cartridge assembly can comprise a magnetic portion (e.g., an embedded stainless-steel disk) that can close a magnetic circuit with the magnet (e.g., permanent electromagnet). Alternatively, or additionally, the coupling mechanism(s) can include mechanical components that latch into or fit into other mechanical components to lock to a cartridge assembly. For example, the coupling mechanism(s) may include latches, tracks, clamps, slots, or other mechanical elements configured to couple the cartridge assembly to the manipulation device. In some embodiments, the coupling mechanism(s) can include a clutch that is configured to allow for mechanical power transfer between the manipulation device and the cartridge assembly. In this manner, the cartridge assembly can be attached to the manipulation device. Additionally or alternatively, the manipulation device can include a cavity, recesses, openings, etc., to receive portions of the cartridge assembly. For instance, the device(s) (e.g., device(s) 544 in FIG. 5) along with guide(s) (e.g., device actuator(s) 534 in FIG. 5) can be configured to fit within the cavity of the manipulation device. In this manner, the cartridge assembly can be mechanically coupled to the manipulation device. In some embodiments, an adaptor is used to drape all components of the robotic system 102 from the cartridge assembly and potential contaminants.

In some embodiments, the robotic arm can include locking mechanisms to lock and unlock the robotic arm. Locking the robotic arm can prevent further movement of the robotic arm. Unlocking the robotic arm can enable it to move as desired. In some embodiments, at 605, the method 600 can include unlocking the robotic arm if the robotic arm is in a locked position (e.g., storage position).

At 606, the method 600 can include moving the robotic arm to position the manipulation device and/or cartridge assembly at a target site. While 606 is shown after 604 in FIG. 6, it can be appreciated that the robotic arm can first be unlocked and positioned so that the manipulation device is positioned near the target site. More specifically, the robotic arm can be positioned so that the manipulation device is positioned/oriented for insertion of the interventional devices (e.g., catheter, guidewire, needle) into a target vessel before the cartridge is attached to the manipulation device. The target site can be a portion of a subject's body on which the medical procedure is to be performed. For example, the target site can be an arm of a patient's body on which the Seldinger technique is to be performed. Moving the robotic arm can include positioning the manipulation device and/or cartridge assembly at a desired location relative to the target site. For example, positioning the manipulation device and/or cartridge assembly can include aligning the cartridge assembly (specifically, a trajectory of the catheter, the guidewire, and/or the needle of the cartridge assembly) with the target blood vessel via a visual aid and/or automatically based on an output from an imaging device as described herein.

In some embodiments, moving the robotic arm can include positioning the cartridge assembly at a desired orientation relative to the target site. For instance, the cartridge assembly can be positioned at an angle with respect to a blood vessel in the target site (e.g., patient's arm, patient's leg, etc.). In some embodiments, the angle can be between about 0 degrees and about 90 degrees, between about 10 degrees and about 80 degrees, between about 20 degrees and about 70 degrees, between about 30 degrees and about 60 degrees, or between about 40 degrees and about 50 degrees with respect to the blood vessel. In some embodiments, the angle can be between about 20 degrees and about 60 degrees. Additionally or alternatively, the cartridge assembly can be positioned at a specific distance from the blood vessel. In some embodiments, the robotic arm can be moved to position the manipulation device such that at least a portion of the manipulation device (e.g., an imaging device such as an ultrasound array) touches the skin of the subject. In some embodiments, moving the robotic arm can include transmitting instructions from an I/O device to the robotic system and/or the robotic arm. For instance, a user can transmit instructions to move the robotic arm via an input device such as a joystick, mouse, keyboard, buttons, etc. Once the robotic arm is moved to position the cartridge assembly at the target site, at 607, the robotic arm can be locked to prevent further movement.

At 610, the method 600 can include controlling the manipulation device to perform a vascular access procedure, e.g., the Seldinger technique. In some embodiments, an I/O device (e.g., I/O device(s) 104 in FIG. 1 or user control 404a in FIG. 4A) communicably coupled to the robotic system can transmit instructions to the robotic system (e.g., a control unit included in the robotic system) so as to control the movement of the robotic arm. For instance, a control unit (e.g., control unit 405 in FIGS. 4A-4B) included in the robotic system (e.g., control unit in the base, control unit in the robotic arm, etc.) can process instructions (e.g., instructions from the I/O device) to control the manipulation device. For example, the needle, the catheter, and/or the guidewire can be actuated based on the instructions. Feedback from sensor(s) (e.g., sensor(s) 106 in FIG. 1) and/or imaging device (e.g., imaging device 536 in FIG. 5) can be used for further subsequent control of the manipulation device. For instance, subsequent control of the actuation of the needle, the catheter, and/or the guidewire can be based on feedback from sensor(s) and/or the imaging device.

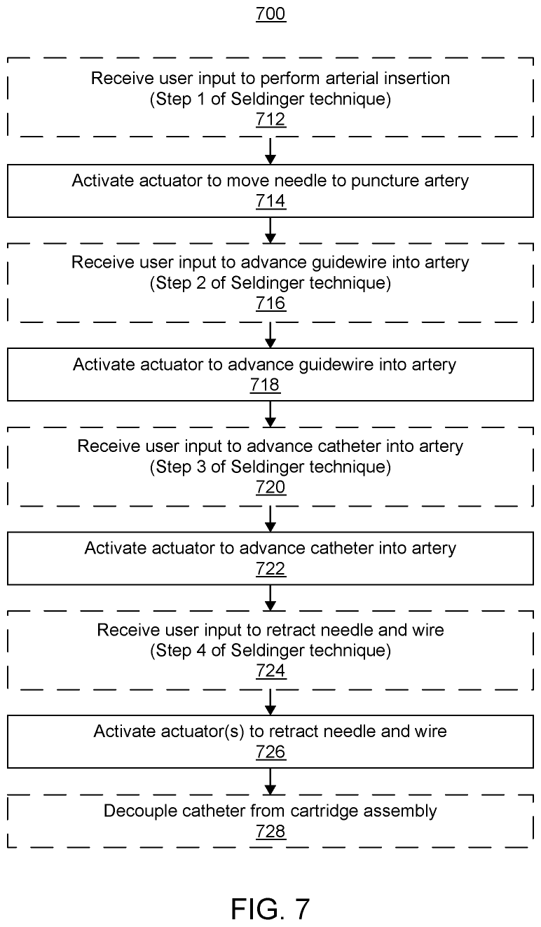

FIG. 7 is a flow diagram illustrating a method 700 of gaining vascular access, for example, by performing the Seldinger technique (e.g., using system 100 in FIG. 1 or any of the other systems and/or components described herein), in accordance with some embodiments. At 712, the method 700 can include receiving user input to access a blood vessel. For instance, the user input can include instructions to perform arterial insertion. In some embodiments, a user can transmit an input to perform the arterial insertion to a robotic system (e.g., robotic system 102 in FIG. 1, robotic system 202 in FIG. 2, etc.) via an I/O device (e.g., I/O device(s) 104 in FIG. 1 or user control 404a in FIG. 4A) that is communicably coupled to the robotic system. The robotic system can be controlled based on the input so as to position and/or orient the robotic system to perform the arterial insertion. For example, the base (e.g., base 203 in FIG. 2), the manipulation device (e.g., manipulation device 230 in FIG. 2), and/or the cartridge assembly (e.g., cartridge assembly 240 in FIG. 2) can be positioned and/or oriented based on the user input such that the cartridge assembly is at a specific distance and/or orientation from the desired artery. While certain steps of the method 700 are described in reference to user inputs, certain steps can receive signals that allow for one or more of the steps of the method 700 to be executed automatically.

At 714, the method 700 can include activating an actuator (e.g., needle actuator of the device actuator(s) 534 in FIG. 5) to move a needle to puncture the artery. In some embodiments, the needle can be included in the cartridge assembly. In some embodiments, the needle can be coupled to a needle guide (e.g., a portion of the needle actuator included in the cartridge assembly) that can include a coupling element such as an adaptor. A cavity in the cartridge device can be configured to fit the needle guide along with the needle. The cartridge assembly can be attached to the manipulation device via an adaptor (e.g., coupling mechanism 538 in FIG. 5). The cartridge assembly can also be attached to the manipulation device via a fastener configured to operably couple the cartridge assembly and the manipulation device. In some embodiments, a linear actuator in the manipulation device is configured to translate a stage, platform, etc., coupled to the cartridge assembly, including the needle.

In response to the input from the user (e.g., via an I/O device) to perform arterial insertion, a linear actuator can be activated. The linear actuator can move along a linear axis. This, in turn, can cause the cartridge assembly or a portion thereof to move, thereby causing at least the needle to move into the artery or target vessel. Accordingly, the needle can be moved and positioned so as to puncture the desired artery. In some embodiments, the needle, the guidewire, and the catheter can be advanced together into the artery. For example, the tip of the needle, the tip of the guidewire, and the tip of the catheter can be aligned with respect to one another, and the entire cartridge (or portion thereof) can be advanced to advance the tips of the three devices into the artery.

In some embodiments, the user can visualize the movement of the needle using a visual aid (e.g., ultrasound images) captured by an imaging device (e.g., imaging device 536 in FIG. 5). For example, a longitudinal view and/or transverse view of the artery can be shown, which can show the tip of the needle (and/or the tip of the catheter and guidewire) as they are advanced into the artery. The visual aid can provide the user with information on the movement of the needle. In some embodiments, the user can visualize the movement of the robotic arm, manipulation device, and/or the cartridge assembly as the needle is being actuated using sensor data obtained from sensor(s) (e.g., sensor(s) 106 in FIG. 1). The user can modify the input to perform arterial insertion based on the visual aid and/or the sensor data.

At 716, the method 700 can include receiving user input (e.g., via an I/O device) to advance the guidewire into the artery. Once the artery has been punctured and the needle positioned in the artery, the user can transmit instructions (e.g., similar to step 712) to advance the guidewire into the artery.

At 718, in response to the instructions at 716, the method 700 can include activating an actuator (e.g., guidewire actuator of the device actuator(s) 534 in FIG. 5) to advance the guidewire distal to the tip of the needle. Similar to the needle, the guidewire can be included in the cartridge assembly. In some embodiments, the guidewire can be coupled to a guidewire guide (e.g., a portion of the guidewire actuator included in the cartridge assembly), which can be attached to the manipulation device via a fastener or an interface, such as a clutch. Additionally, and/or alternatively, the guidewire may not be attached to a guidewire guide.

In response to the user input (e.g., via an I/O device) to advance the guidewire, a linear actuator (e.g., a portion of the guidewire actuator included in the manipulation device and/or the cartridge assembly) to actuate the guidewire can be activated. The linear actuator can cause movement along a linear axis. This, in turn, can cause the guidewire guide and/or the guidewire to move along the linear axis. Accordingly, the guidewire can be advanced distal to the needle and to the desired location in the artery. The needle and the catheter can be held stationary as the guidewire is advanced into the artery. In some embodiments, the user can visualize the movement of the guidewire using a visual aid captured by the imaging device. In some embodiments, the user can visualize the movement of the robotic arm, manipulation device, and/or the cartridge assembly as the guidewire is being advanced using sensor data obtained from the sensor(s). The user can modify the input to advance the guidewire based on the visual aid and/or the sensor data.

At 720, the method 700 can include receiving user input (e.g., via an I/O device) to advance the catheter into the artery. In some embodiments, the user can transmit instructions (e.g., similar to step 712) to advance the catheter into the artery.

At 722, in response to the instructions at 720, the method 700 can include activating an actuator (e.g., catheter actuator of the device actuator(s) 534 in FIG. 5) to advance the catheter over the guidewire and distal to the tip of the needle. Similar to the needle, the catheter can be included in the cartridge assembly. In some embodiments, the catheter can be coupled to a catheter guide (e.g., a portion of the catheter actuator included in the cartridge assembly) and can be attached to the manipulation device via a fastener or an interface, such as a clutch. Additionally, or alternatively, the catheter may not be attached to a catheter guide.

In response to the user input (e.g., via an I/O device) to advance the catheter, a linear actuator (e.g., a portion of the catheter actuator included in the manipulation device and/or the cartridge assembly) to actuate the catheter can be activated. The linear actuator can cause movement along a linear axis. This, in turn, can cause the catheter guide and/or the catheter to move along the linear axis. Accordingly, the catheter can be advanced to the desired location in the artery. In some embodiments, the user can visualize the movement of the catheter using a visual aid captured by the imaging device. In some embodiments, the user can visualize the movement of the robotic arm, manipulation device, and/or the cartridge assembly as the catheter is being advanced using sensor data obtained from the sensor(s). The user can modify the input to advance the catheter based on the visual aid and/or the sensor data. A variation of the system can be configured such that the linear actuators provide a vibratory effect when advancing a needle or other component (e.g., a linear resonant actuator). Alternatively, or in combination, the vibratory effect can be obtained using a secondary motor (including, but not limited to, an eccentric rotating mass, a vibratory motor, etc.)

At 724, the method 700 can include receiving user input (e.g., via an I/O device) to retract the needle and the guidewire. At 726, in response to the instructions at 724, the method 700 can include activating the actuator(s) to retract the needle and the guidewire. For example, at least one linear actuator can be activated to retract the needle and the guidewire along the linear axis. In some embodiments, a first linear actuator configured to retract the entire cartridge assembly can be activated so that the needle and the guidewire can be retracted, while a second linear actuator can be actuated to maintain a position of the catheter while the entire cartridge is being retracted. In some embodiments, the user can visualize the movement of the retraction of the needle and the guidewire using a visual aid captured by the imaging device. In some embodiments, the user can visualize the movement of the robotic arm, manipulation device, and/or the cartridge assembly as the needle and/or the guidewire is being retracted using sensor data obtained from the sensor(s). The user can modify the input to retract the needle and/or the guidewire based on the visual aid and/or the sensor data.

At 728, the method 700 can include decoupling the catheter from the cartridge assembly. In some embodiments, the catheter can be detached from the catheter guide without releasing the catheter guide from the cartridge assembly. For example, the user can manually decouple the catheter from the catheter guide without decoupling the catheter guide from the cartridge assembly. For example, the catheter may be attached to the catheter guide via a pin assembly, and the catheter may be decoupled from the cartridge by removing at least one pin. The user can wait to detach the catheter until after the needle and the guidewire have been retracted.

Although in FIG. 7, the sequence of steps to gain access to a blood vessel is described as activating an actuator to advance the needle followed by activating an actuator to advance the guidewire and subsequently activating an actuator to advance the catheter, it should be readily understood that the sequence of steps to gain access to a blood vessel using the system and methods described herein can be performed in any suitable permutations and combinations. For example, in some embodiments, one or more actuators can be activated to advance the needle, the catheter, and the guidewire simultaneously. Once the needle punctures the desired blood vessel (e.g., artery), the guidewire can be advanced distal to the needle to a desired position in the blood vessel. The catheter can then be advanced over the guidewire to the desired position in the blood vessel. In some embodiments, after advancing the guidewire but before advancing the catheter to the desired position in the blood vessel, the needle can be retracted slightly (e.g., moved proximally by a small distance) so that advancing the catheter may be atraumatic to the subject. Alternatively, one or more actuators can be activated to align the needle tip and the distal end of the catheter. The needle and the catheter can be advanced simultaneously to a desired blood vessel. Once the needle punctures the desired blood vessel, the guidewire can be advanced through the puncture to a desired position in the blood vessel. The catheter can then be further advanced to the desired position in the blood vessel. As discussed above, these are a few examples to illustrate various permutations and combinations for accessing a blood vessel using the systems and methods described herein.

In some embodiments, if the size of the blood vessel is large (e.g., central vein), a second catheter can be advanced over the first catheter in order to perform the medical procedure. Put differently, one or more actuators can advance the needle, the guidewire, and the catheter to a desired position in the desired blood vessel. Then, the needle and the guidewire can be retracted from the blood vessel. Another guidewire can be advanced (e.g., manually and/or autonomously) through the catheter already positioned in the desired location. A second catheter that is bigger in size than the already positioned catheter can be advanced through the guidewire. In this manner, the second larger catheter can be positioned through the first catheter in order to perform the medical procedure. In some embodiments, one or more dilators can be used before positioning either the first catheter (e.g., catheter advanced using actuator(s) in the manipulation device and/or cartridge assembly) and/or the second catheter (e.g., catheter that is larger than the first catheter and is advanced through the first catheter) during the medical procedure.

In some embodiments, method 700 as described herein can be performed autonomously and/or semi-autonomously. Accordingly, one or more steps of receiving user input (e.g., 712, 716, 720, 724) can be optional, and systems and devices described herein can be configured to automatically proceed from actuating one component to the next based on confirmation that a first step has been completed. Such confirmation can be determined via sensor data (e.g., via sensor(s) 106) and/or imaging data (e.g., via imaging device 536). In some embodiments, one or more steps may be performed without user input, while other steps may be performed with user input.

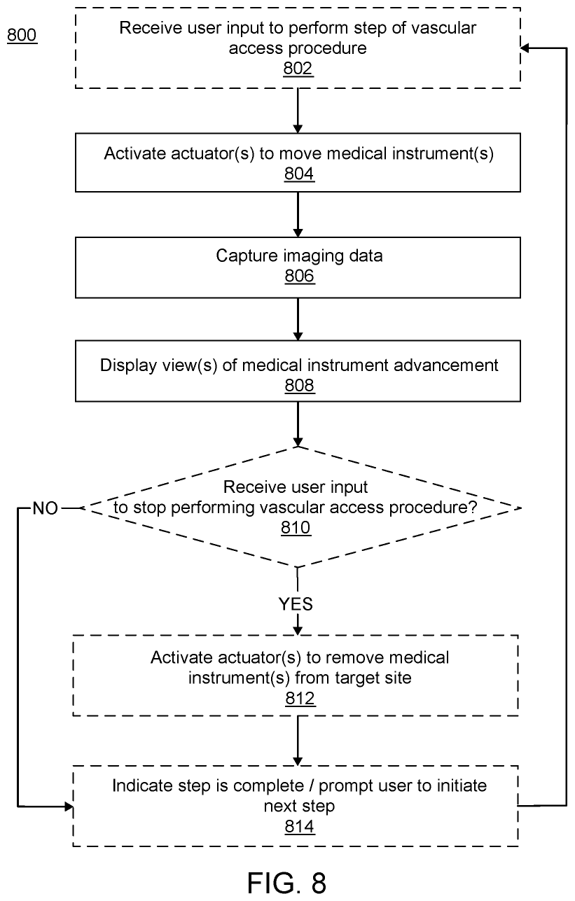

FIG. 8 is a flow diagram illustrating a method 800 of using visual aid and/or sensor data to perform a medical procedure (e.g., using system 100 in FIG. 1), in accordance with some embodiments. At 802, the method 800 can include receiving user input to perform a step of the medical procedure (e.g., vascular access procedure). For example, the user input can include instructions to perform arterial insertion (e.g., step 712 in FIG. 7), advance a guidewire into a blood vessel (e.g., step 716 in FIG. 7), advance a catheter into a blood vessel (e.g., step 720 in FIG. 7), and/or retract a needle and the guidewire from the blood vessel (e.g., step 724 in FIG. 7). In some embodiments, the user can provide a plurality of user inputs corresponding to different stages of the vascular access procedure. In some embodiments, the method 800 can start automatically without a user input. In some embodiments, the user input can be received in response to a position of a vascular access device being confirmed. For example, when the position of the vascular access device is in a desired location and/or orientation, a user can generate a user signal to initiate a vascular access procedure.

At 804, the method 800 can include activating actuators to move medical instruments based on the user input. For example, activating actuators can include activating actuators within a robotic system (e.g., robotic system 202 in FIG. 2 and/or other robotic systems described herein) to move transport elements (e.g., transport element 414 in FIG. 4A) included in the robotic system based on the user input. This in turn can cause a robotic arm (e.g., robotic arm 220 in FIG. 2 and/or other robotic arms described herein), a manipulation device (e.g., manipulation device 230 in FIG. 2 and/or other manipulation devices described herein), and/or a cartridge assembly (e.g., cartridge assembly 240 in FIG. 2 and/or other cartridge assemblies described herein) to be positioned at a desired location from a target site (e.g., subject's body part, such as an arm). In some embodiments, activating actuators can include activating device actuators (e.g., device actuator(s) 534 in FIG. 5) to advance (e.g., steps 714, 718, and 722 in FIG. 7) and/or retract (e.g., step 726 in FIG. 7) the needle, the catheter, and/or the guidewire into/from a blood vessel based on the user input.

At 806, the method 800 can include capturing visual aid and/or sensor data as the step of the vascular access procedure is being performed. For example, the manipulation device can include an imaging device (e.g., imaging device 536 in FIG. 5) to capture visual aid (e.g., ultrasound images) of the needle, the catheter, and/or the guidewire (collectively, device(s) 544 in FIG. 5) as the device(s) are being advanced into and/or retracted from the blood vessel. In some embodiments, the robotic system can be communicably coupled to a sensor (e.g., sensor(s) 106 in FIG. 1), such as a camera to capture images of the robotic system, the manipulation device, and/or the cartridge assembly as the step of the vascular access procedure is being performed.

At 808, the visual aid and/or the sensor data can be displayed on an I/O device (e.g., I/O device(s) 104 in FIG. 1 or user control 404a in FIG. 4A). For example, ultrasound images and/or images from cameras can be displayed on a display. At 812, if a user input to stop performing the step of the medical procedure is received (e.g., at step 810), the method 800 can include activating the actuators to remove the medical instruments from the target site. For example, the needle, the catheter, and/or the guidewire can be retracted from the blood vessel by activating the device actuators. Additionally or alternatively, the robotic system, the manipulation device, and/or the cartridge assembly can be moved away from the subject by activating the actuators coupled to the transport elements. In some embodiments, the cartridge assembly can be detached from the manipulation device.

At 814, the method 800 can include indicating to the user that the step of the vascular access procedure is complete. For example, the display can output visual, audio, and/or haptic outputs to indicate that the step of the medical procedure is complete. In some embodiments, the display can also prompt the user to initiate the next step, thereby repeating the steps of method 800. If the user input to stop performing the step of the medical procedure is not received at step 810, the method 800 can include, at 814, indicating to the user that the step of the vascular access procedure is complete and prompting the user to initiate the next step of the vascular access procedure.

FIG. 9 is a flow diagram illustrating a method 900 of retracting vascular access components (e.g., needle and catheter), in accordance with some embodiments. In some embodiments, the method 900 can be implemented during a vascular access procedure and after a catheter has been successfully inserted into a blood vessel. At 902, the method 900 includes setting a skin position based on a position associated with a cartridge (similar to any of the cartridges described herein). In some embodiments, setting the skin position can include setting the position of the skin based on the manipulation device or portion thereof (e.g., an imaging device integrated with or supported by the manipulation device) engaging the skin. For example, the manipulation device and/or a robotic arm supporting the cartridge can include a sensor (e.g., a pressure sensor) that can indicate that the manipulation device is engaging the skin. In some embodiments, setting the skin position can occur before the vascular access procedure or during the vascular access procedure. Specifically, setting the skin position can occur prior to accessing the blood vessel. In some embodiments, a portion of the vascular access procedure can occur between 902 and 904. For example, a portion of the method 700 and/or the method 800 can occur after 902 and before 904. In some embodiments, the method 900 only continues to 904 after the catheter has been inserted into the blood vessel.

At 904, the method 900 includes activating an actuator associated with the catheter, while retracting the cartridge (e.g., and thus the needle and the guidewire), to maintain the catheter position (e.g., in the blood vessel). In some embodiments, the actuator is activated in an opposite direction and at an equal speed to the retraction of the cartridge to prevent the cartridge from being retracted out of the blood vessel as the needle is retracted via the cartridge. In some embodiments, the cartridge is retracted a predetermined distance away from the skin position. In some embodiments, the predetermined distance (e.g., threshold distance) may be associated with a distance where the likelihood of the needle accidentally puncturing the skin is low. At 906, the method 900 includes stopping the actuator and the cartridge, based on the needle being retracted at least the threshold distance from the skin. At 908, the method 900 includes activating an actuator associated with the guidewire to retract the guidewire. Once the needle and the guidewire are retracted, only the catheter remains in the blood vessel, which can be used as desired. In some embodiments, the method 900 can include detaching the catheter from the cartridge.

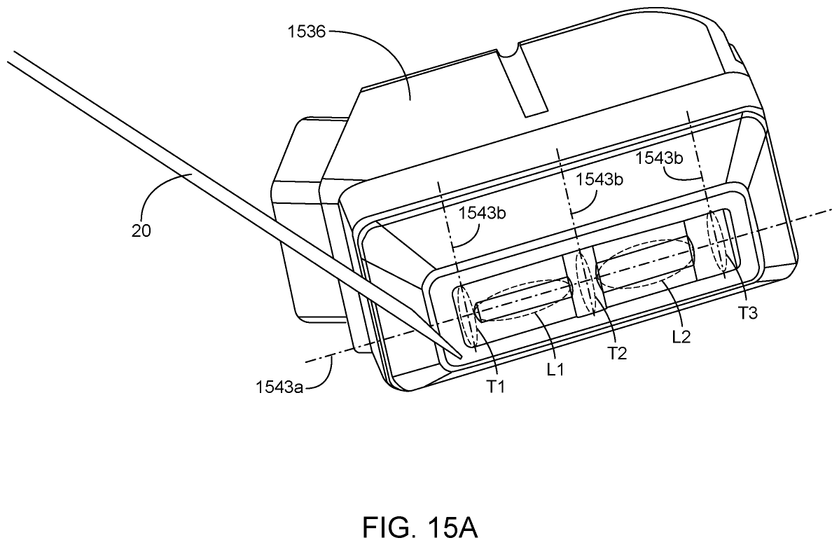

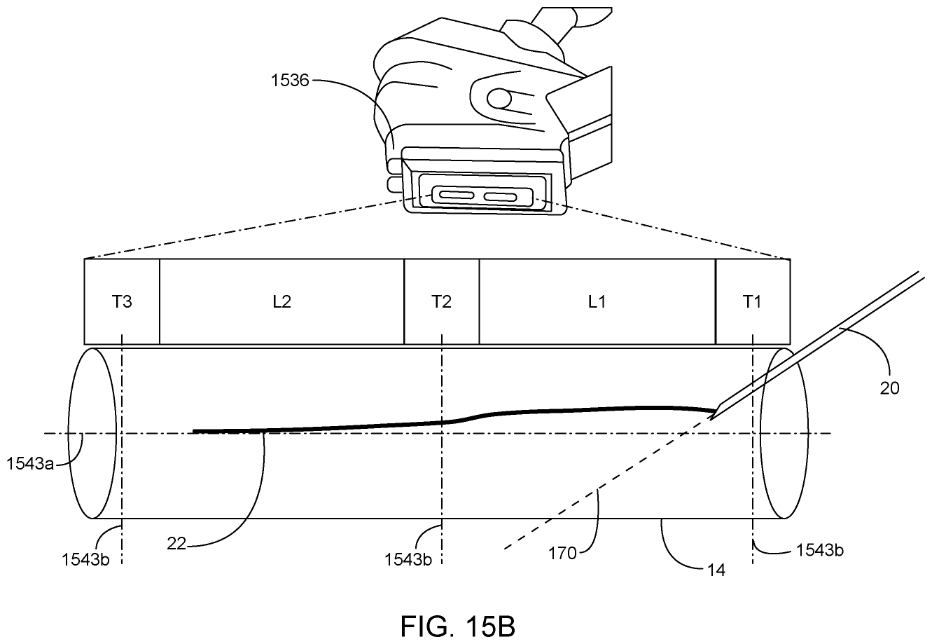

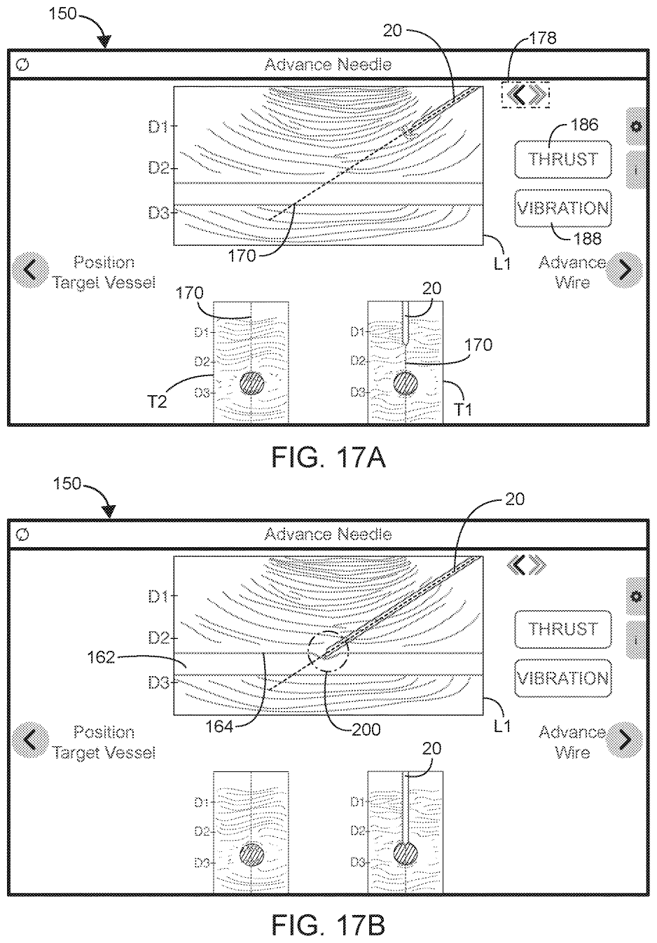

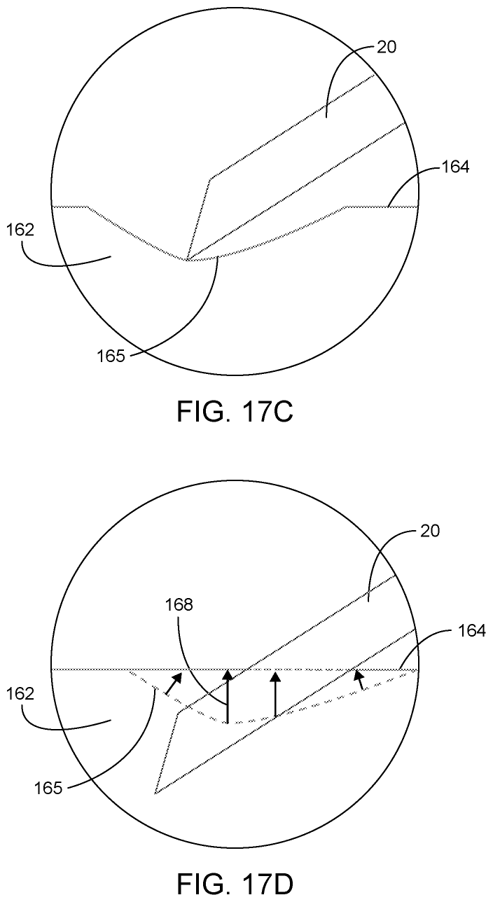

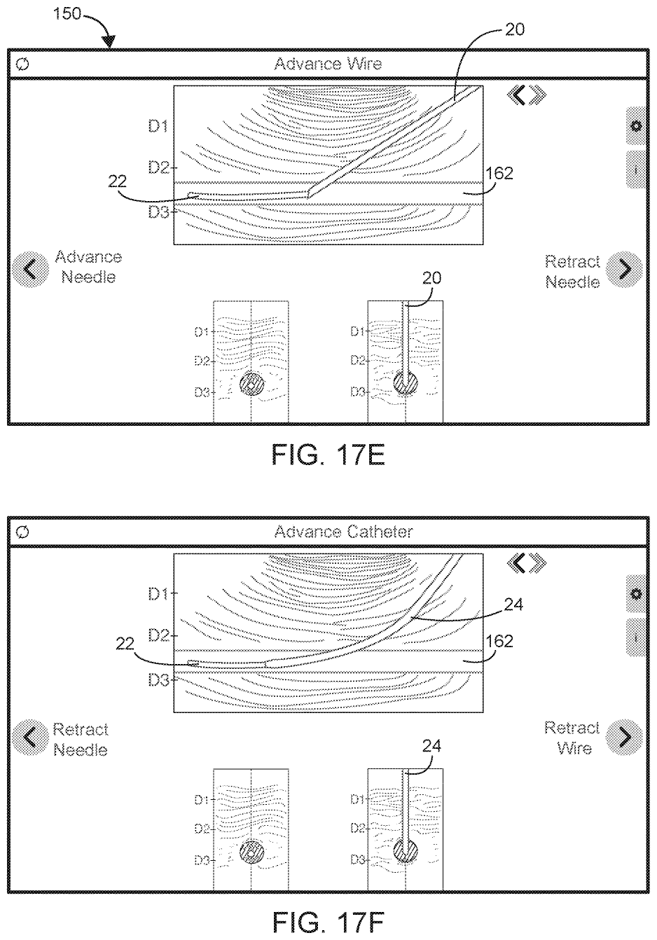

FIG. 17 is a flow diagram illustrating a method 1700 for aligning multiple views of a blood vessel, in accordance with some embodiments. The method 1700 can be executed automatically (e.g., by a control unit such as the control unit 405 of FIGS. 4A-4B) and/or by a user during a vascular access procedure. The method 1700 aligns the views of the blood vessel so that a manipulation device (e.g., similar to manipulation devices described herein) is in a desired position and orientation to execute a vascular access procedure. In some embodiments, the method 1700 uses information (e.g., ultrasound signal, views, etc.) from an imaging device having a plurality of transverse views, such as the imaging device 1536 of FIG. 15A.

At 1702, the method 1700 optionally includes positioning an imaging device (including a transducer array) of a robotic system near a target structure (e.g., blood vessel). For example, a robotic system can automatically, or a user can, position the imaging device to substantially engage the skin of the patient near the target structure. Once the imaging device is positioned, it can begin providing at least a first transverse view and a second transverse view of the target structure, where the first transverse view and the second transverse view are separated by a distance.

At 1704, the method 1700 includes identifying a first portion of the target structure in the first transverse view captured by the imaging device. The first portion can be a first radial cross-section of the target structure. In some embodiments, identifying can include using one or more machine learning models, algorithms, etc., to identify the target structure in the anatomy of the patient as described in reference to the anatomy alignment 407b of FIG. 4B. In some embodiments, determining the target structure can include determining a centroid of the first radial cross-section. At 1706, the method 1700 includes aligning the centroid of the first portion with a first marker. The first marker may be a visual aid indicating where the needle (or catheter and/or guidewire) would be inserted. Aligning the centroid of the first portion with the first marker allows for confirmation that the needle (or catheter and/or guidewire) is expected to be inserted at the centroid, thus reducing the chance of an undesirable puncture.

At 1708, the method 1700 includes identifying a second portion of the target structure in the second transverse view captured by the imaging device. Similar to 1704, the second portion of the target structure can be a second radial cross-section of the target structure. In some embodiments, identifying can include using one or more machine learning models, algorithms, etc., to identify the target structure in the anatomy of the patient as described in reference to the anatomy alignment 407b of FIG. 4B. In some embodiments, determining the target structure can include determining a centroid of the second radial cross-section. At 1710, the method 1700 includes aligning the centroid of the second portion with a second marker. Similar to the first marker, the second marker may be a visual aid indicating where at least one of the catheter, the guidewire, or the needle would be inserted. Aligning the centroid of the second portion with the second marker allows for confirmation that the catheter, the guidewire, and/or the needle is expected to be inserted at or advance through the vessel at the centroid, thus reducing the chance of injury to the vessel.

At 1712, the method 1700 optionally includes confirming that the first and second markers are aligned with the centroid of the first portion and the centroid of the second portion, respectively. Confirming that both the first and second markers are aligned with the centroids confirms that the catheter, the guidewire, and/or the needle would be inserted into the target structure or advanced within the target structure toward a center of the vessel, e.g., between the plane of the first transverse view and the second transverse view. Aligning both the transverse views increases the safety of the vascular access procedure when compared to only using a single transverse view, as the multiple transverse views can confirm that the needle is positioned as desired, not only for puncturing into the target structure but also once the needle is inside the target structure. In some embodiments, the method 1700 can include additional identification and alignment steps for additional transverse views. For example, if the target structure is below a predetermined threshold below the skin, additional transverse views may be desired.

At 1714, the method 1700 optionally includes generating at least one confirmation signal, based on the confirmation at 1712. In some embodiments, the confirmation signal can be a display signal, an audio signal, a vibration, a visual signal, and/or the like to indicate to a user that the manipulation device is in a desired position and orientation. At 1716, the method 1700 optionally includes engaging a brake to lock a position of the robotic system, based on the confirmation at 1712. The brake can be a brake associated with a cart, the manipulation device, and/or a robotic arm. Locking the position can allow for the target vessel and the robotic system to be in the desired position during the vascular access procedure. In some embodiments, the transverse views and/or the longitudinal views can be monitored during the vascular access procedures to determine if an alteration to the position of the robotic system is desired.

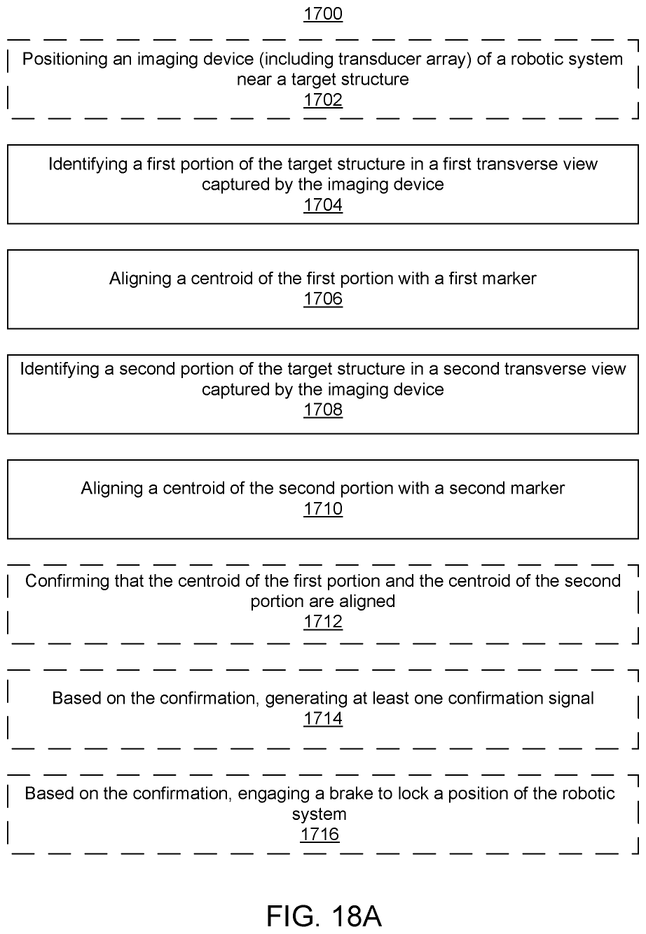

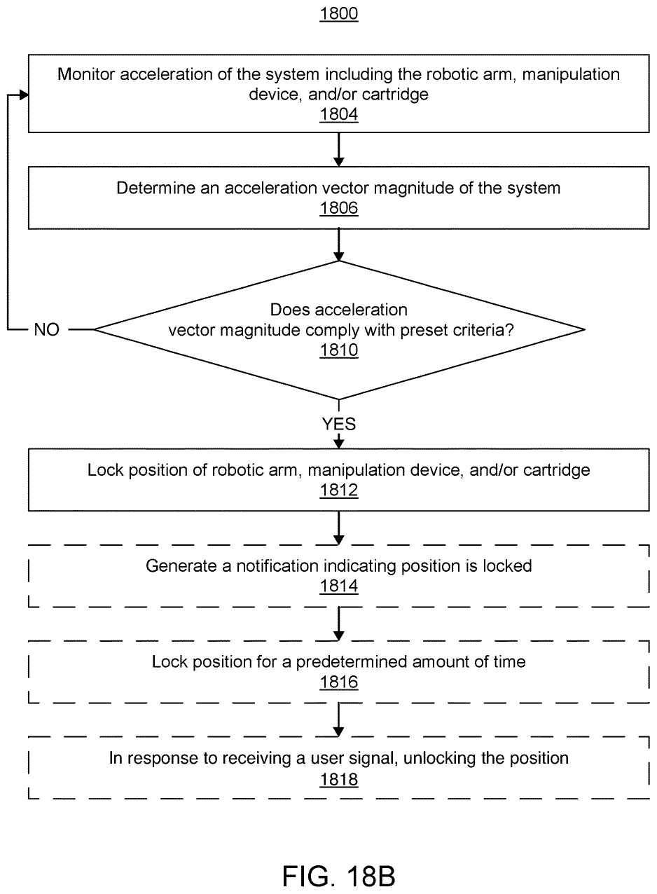

FIG. 18 depicts a method 1800 for actuating a ball joint brake of a robotic arm, in accordance with some embodiments. In some embodiments, the ball joint is controlled via a control unit (e.g., structurally and/or functionally similar to the control unit 405 of FIGS. 4A-4B) during operation of the robotic arm. Method 1800 can be operated while the robotic arm is being used during a procedure to decrease the likelihood of the robotic arm causing undesirable tissue damage during any erratic motion of the robotic arm.

At 1804, the method 1800 includes monitoring accelerometer data of the system, including the robotic arm, manipulation device, and/or the cartridge. For example, the accelerometer data can be from a sensor on at least a portion of the robotic arm and/or a sensor located on the manipulation device. In some embodiments, the accelerometer data can be preprocessed (e.g., filtered, normalized, etc.). At 1806, based on the acceleration data, an acceleration vector magnitude is determined.

At 1810, the method 1800 includes determining whether the acceleration vector magnitude exceeds a predefined acceleration threshold. In some embodiments, the predefined acceleration threshold is between about 0.1 g and about 3 g, including, for example, 0.1 g, 0.2 g, 0.3 g, 0.4 g, 0.5 g, 1 g, 2 g, 3 g, inclusive of all ranges and values therebetween. The predefined acceleration threshold may be associated with an acceleration that may indicate that the robotic system is moving in an undesired manner. For example, the undesired manner can include an indication that the system was bumped, hit, etc. As another example, the undesired manner can indicate that a needle has contacted undesired anatomy, such as bone. If the acceleration vector magnitude is not greater than the predefined acceleration threshold, the method 1800 returns to 1804, wherein acceleration is further monitored. If the acceleration vector magnitude is greater than the predefined acceleration threshold, the method 1800 continues to 1812.

At 1812, the method 1800 includes actuating brakes to lock the position of the robotic arm. Locking the position of the robotic arm can decrease or prevent the likelihood of the robotic arm impacting the patient and/or the surrounding space. In some embodiments, locking the robotic arm includes actuating magnetic brakes in a ball joint to lock the ball joint in place. In some embodiments, the brakes can lock automatically when the robotic system loses power. At 1814, a notification is generated indicating that the brakes are actuated. The notification can be sent to a user so that the user can be aware of a potential issue and to indicate that the system is locked. In some embodiments, 1814 is optional. As in 1816, the brakes may be locked for a predetermined amount of time. In some embodiments, the brakes may unlock after the predetermined amount of time. In some embodiments, 1816 is optional. At 1818, the method 1800 optionally includes unlocking the brakes in response to receiving a user signal. For example, if a user indicates on an input that the issue has been resolved and that the operation may proceed, the brakes may be unlocked.

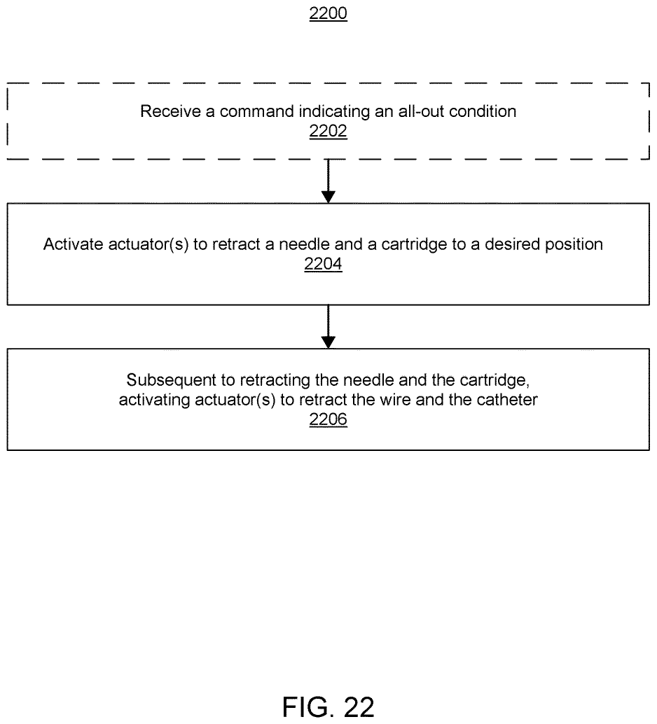

FIG. 22 illustrates a method 2200 for retracting vascular access components, in accordance with some embodiments. For example, the method 2200 can be used for an all-out condition as described above. The all-out condition can be activated when desired by a user and/or automatically by a system (e.g., in response to a fault condition, collision, unexpected operation, and/or movement, etc.). At 2202, the method 2200 includes receiving a command indicating an all-out condition. Once the all-out condition is received, or if a full retraction is otherwise desired, at 2204, the method 2200 includes activating actuator(s) (e.g., structurally and/or functionally similar to any of the actuators described herein such as the device actuator(s) 534 of FIG. 5) to retract the cartridge (including needle, guidewire, and catheter) to a desired position. In some embodiments, the desired position may be a predetermined distance away from the skin of the patient so as not to cause inadvertent damage from the needle. At 2206, the method 2200 includes activating actuator(s) to retract the guidewire and catheter subsequent to retracting the cartridge. Retracting the needle via retraction of the entire cartridge (i.e., retraction of the needle with the guidewire and the catheter) can allow for a decreased likelihood of inadvertent damage from the needle to the guidewire and/or catheter during retraction of the guidewire and the catheter.

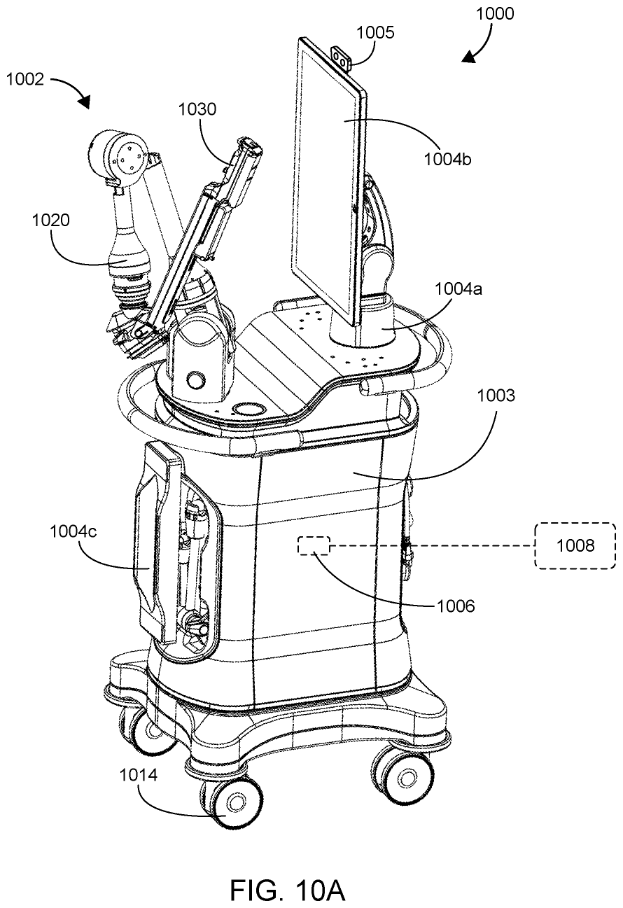

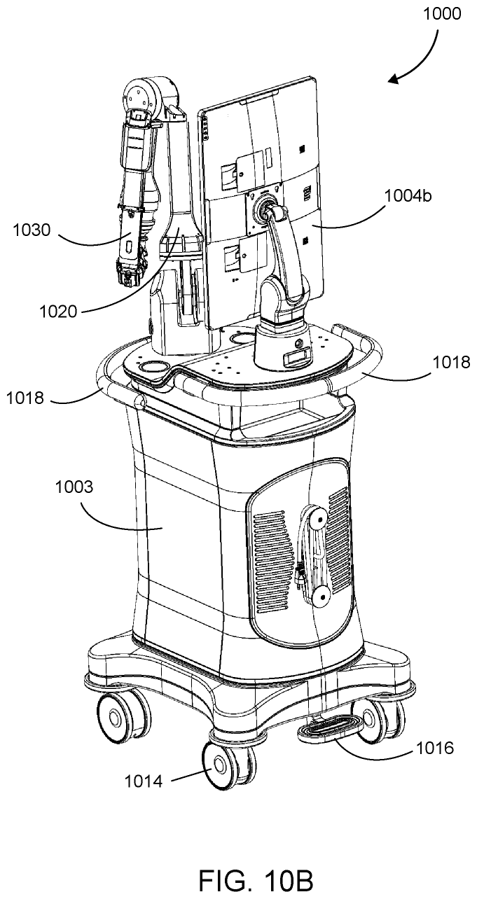

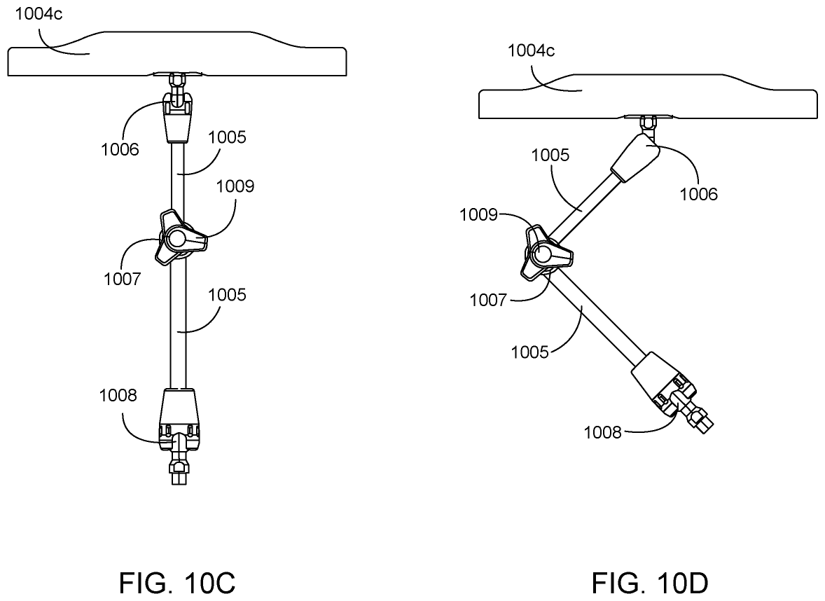

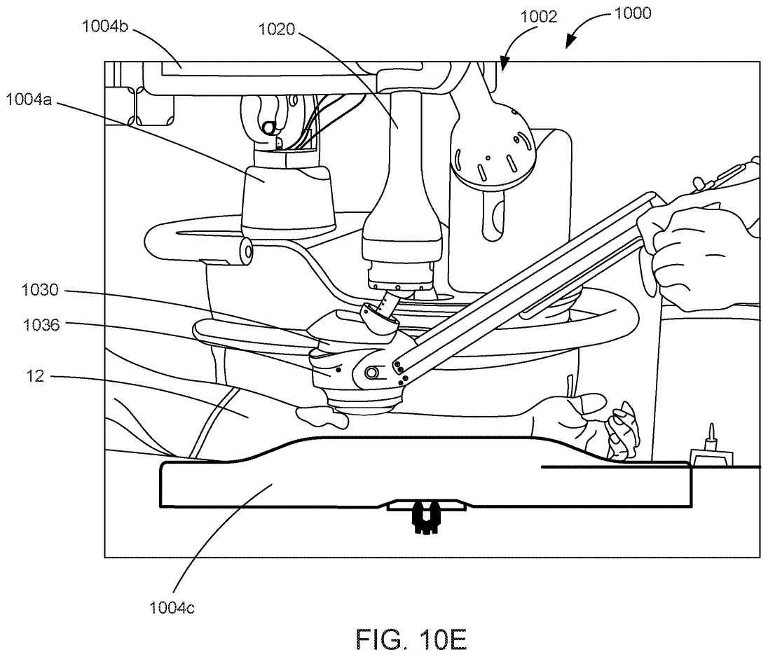

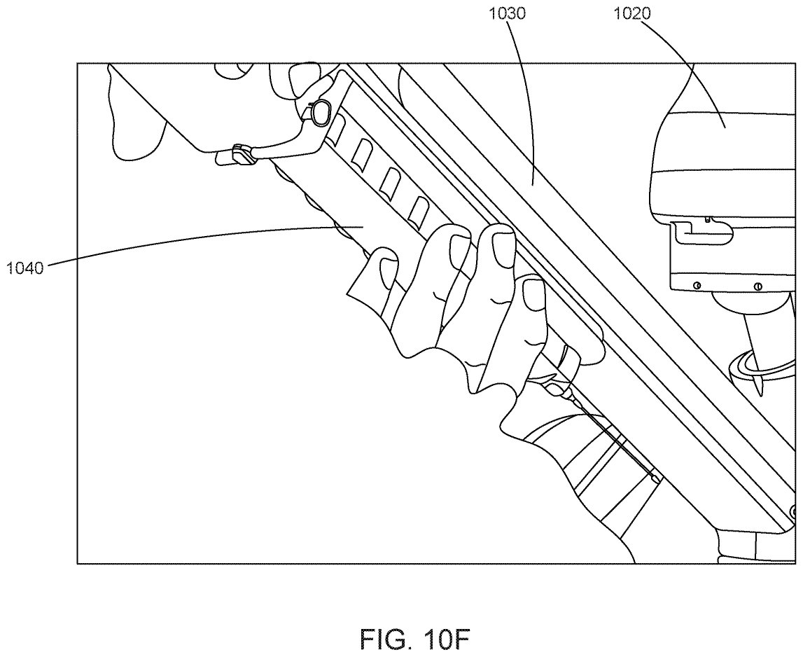

FIGS. 10A-10D show a variation of a vascular access system 1000 (e.g., structurally and/or functionally similar to system 100 in FIG. 1). The vascular access system 1000 can transition between multiple configurations, including but not limited to a first configuration (e.g., a storage or transport configuration) and a second configuration (e.g., for performing a vascular access procedure). As seen in FIG. 10A, which depicts a variation of a vascular access system 1000 in the storage configuration, this vascular access system 1000 includes a robotic system 1002 with a base 1003 (e.g., structurally and/or functionally similar to base 203 in FIG. 2 and other bases described herein), robotic arm 1020 (e.g., structurally and/or functionally similar to robotic arm 220 in FIG. 2 and other robotic arms described herein), and manipulation device 1030 (e.g., structurally and/or functionally similar to manipulation device 230 in FIG. 2 and other manipulation devices described herein).

The base 1003 can be movable and configured to be lockable in place. For example, the base 1003 can include transport elements 1014 (e.g., structurally and/or functionally similar to transport elements 414 in FIG. 4A). As an example, the transport elements 1014 can be swivel casters with lockable wheels. The swivel casters can provide the base 1003 with three degrees of freedom. For example, the swivel casters can provide translations along a floor or other surface. Planar and rotational movement can enable the base 1003 to be positioned relative to a subject's (e.g., patient on whom the medical procedure is to be performed) arm. In some embodiments, a locking mechanism (e.g., locking mechanism(s) 412 in FIG. 4A) can lock the transport elements 1014 to lock a position of the base 1003 during the medical procedure. For example, in some embodiments, locks may be engaged (e.g., automatically and/or manually) before and/or during the medical procedure, such as upon the base being positioned at an appropriate position relative to a target (e.g., arm, leg, etc.) of a patient on whom the medical procedure is to be performed. The vertical height of the base 1003 can be adjustable or fixed to a predetermined height.

FIG. 10A also shows that the system 1000 can include one or more control units/processors 1006 that control the robotic assembly and user interface (described below). The processors/control units 1006 can be located in the base 1003 and/or in the display 1004b. In addition, the system 1000 and/or processors 1006 can be configured to engage with an external network for processing or a cloud network 1008 through any wired or wireless connection. In some variations, the cloud network 1008 can supplement the system by providing image processing for an artificial intelligence-enhanced display of the images shown on the display. The external/cloud network can also be used for remote monitoring of the procedure as an additional safety feature. In additional variations of the system, all processing is performed within the system 1000 itself.

As noted here, the transport elements 1014 can have a normally locked configuration, where movement of the base 1003 requires an affirmative act by an individual using the user interface of the system 1000 and/or one or more separate mechanical/electromechanical switches. FIG. 10B illustrates the system 1000 of FIG. 10A from a different perspective, showing a side of the system 1000 opposite to the user side, where the user side allows a user to engage the manipulation device 1030, the robotic arm 1020, and the display 1004b. FIG. 10B illustrates the base 1003 having a locking mechanism 1016 that, in this variation, comprises a foot-actuated pedal. In the variation shown in FIG. 10B, the locking mechanism 1016 is positioned opposite to the operator side (e.g., the side with the robotic system 1002, support surface 1004c, etc.), in an effort to reduce the likelihood of inadvertent actuation, and/or unintentional movement of the base 1003 or system 1000, either by the operator or patient, prior to or during a procedure. However, variations of the system can include the locking mechanism 1016 on the operator side or on multiple sides. The illustrated variation also shows one or more handles or other manipulation structures 1018 positioned opposite the operator side, which allows positioning of the system 1000 from the side with the locking mechanism 1016 and locking the system 1000 in place. In additional variations, a locking mechanism or additional locking mechanisms can be located on the manipulation structure.

Additional variations of the system 1000 can include multiple locking mechanisms 1016 positioned on any side or multiple sides of the base 1003. Alternatively, or in combination, the locking state of the base 1003 can be managed from a user interface on the display 1004b. In each variation, the locking system/state controlled by the locking mechanism 1016 can include one or more locking states to assist in positioning of the base 1003 and/or system 1000. For example, the locking system can include a simple lock/unlock position. Alternatively, or in combination, the locking system can include, at least, a neutral state, a steer state, and a locking state. The neutral state allows free movement of the transport elements 1014 to relocate or position the base 1003. The steer state can control one or more of the transport elements 1014, to steer or direct the system 1000/base 1003. For example, in one variation, when in the steer state, an operator can rotate the base 1003 from the operator side without translating the base 1003. The lock state can prevent movement of the base 1003 and/or transport elements 1014. It is noted that variations of the system 1000 can include a locking system that controls each transport element 1014. Additionally, the system 1000 can be configured to default to the locked state when powered or unpowered, and where each non-locked state is time-limited such that the system reverts to the locked state after a period of time.

In some embodiments, the base 1003 can have one or more I/O device(s). For example, a display 1004b (e.g., structurally and/or functionally similar to display 404b in FIG. 4A) can be mechanically coupled to the top surface of the base via a display support 1004a. Clearly, the display 1004b and/or other I/O devices can be coupled to any portion of the base. The display 1004b can provide a user interface that provides visual aid and/or visual feedback, including longitudinal and transverse ultrasound views of a blood vessel of the subject. In some embodiments, the user interface of the display 1004b can provide state and status information on the task being performed during the medical procedure.

Variations of the system 1000 include a robotic arm 1020 that is attached to, integrated with, and/or otherwise coupled to the base 1003. While the illustration shows the robotic arm 1020 coupled to the top of the base 1003, variations of the system allow for coupling the robotic arm 1020 to any part of the base. The robotic arm 1020 is configured to allow a caregiver to position and orient a manipulation device 1030 in three-dimensional space for engaging a target of a patient. Variations of the system 1000 include a robotic arm 1020 that is counterbalanced but passive, such that the arm segments do not move without being positioned by the user. The counterbalancing of the robotic arm 1020 allows the distal end (the end coupled to the manipulation device 1030) to freely float when the system 1000 is in an unlocked configuration. In additional variations and as discussed below, the passive robotic arm 1020 can include one or more motors that fine-tune alignment of the manipulation device 1030 and/or imaging device 1036 after the caregiver approximates positioning of the robotic arm 1020 to access the vessel. Additionally, and as discussed below, the robotic arm 1020 can be configured to alter motion (e.g., increase dampening or increase resistance to movement) when the system 1000 detects that the manipulation device 1030 is approaching or in an acceptable position to deploy the needle. In yet additional variations of the system 1000, the robotic arm 1020 can be fully automated.

The base 1003 can include a patient support 1004c coupled to the base 1003 and adjacent to the robotic arm 1020. While the patient support 1004c illustrated in FIG. 10A couples to a side of the base 1003, variations of the system 1000 can include one or more patient supports 1004c coupled to any side or top of the base 1003. FIGS. 10C and 10D illustrate examples of a patient support 1004c coupled to one or more support arms 1005 when moved away from the storage position shown in FIG. 10A. The patient support 1004c is configured to extend, fold out, and/or the like to allow for a patient to rest a desired anatomical region on the support 1004c.

The patient support 1004c can include a surface with a channel or other concave surface, which receives an arm or a leg of an individual. Accordingly, in variations of the system 1000, the side walls, length, or other feature of the patient support surface 1004c can be adjustable to accommodate different sizes or different body parts. FIGS. 10C and 10D show a variation of a patient support surface 1004c coupled to two support arms 1005; however, any number of support arms is within the scope of this disclosure. The variation shown in FIG. 10C shows an upper support arm 1005 coupled to the patient support surface 1004c through a joint (e.g., a ball joint) on a first end and then coupled to the lower support arm 1005 through a second joint (e.g., a ball joint). The opposite end of the lower support arm 1005 is coupled to the base (not shown in FIGS. 10C and 10D) using another joint 1008 (e.g., a ball joint). The joints 1006, 1007, 1008 can allow for rotation or translation of the arm to provide for flexibility when positioning the patient support surface 1004c in free space. While each joint 1006, 1007, 1008 can be individually lockable, the illustrated variation provides for a single actuator or knob 1009 that allows for locking of the patient support surface 1004c and supporting structure (e.g., 1005-1009) in place once the operator determines a desired position/orientation of the patient support surface 1004c. As described above, the supporting structure can be counterbalanced to assist in positioning the support surface 1004c. In addition, the ability to lock the structure using a single knob/actuator 1009 increases the ease of setup of the system 1000.

In some embodiments, the system may include an additional image sensor, such as one or more stereo cameras, 1005, to enable precise motion of the robotic arm 1020 and/or manipulation device 1030. The additional image sensor 1005 can allow precise targeting and/or access to the vascular portion. For instance, the feedback from the additional image sensor can control the manipulation device 1030 and/or the robotic arm 1020 in a more precise manner. In some embodiments, proximity sensor(s) may be attached to, coupled to, and/or otherwise mounted on the base 1003 to enable precise motion and targeting. In some variations, the image sensor 1005 can be embedded within the display 1004b.

FIG. 10E illustrates an example of a robotic system 1000 of FIG. 10A with an arm 12 of a patient resting on a patient support 1004c that is moved from a storage configuration into a deployed configuration to support the patient's arm 12. As shown, this permits an imaging device 1036 of a manipulation device 1030 to engage the arm 12 of the patient. In some embodiments, when the manipulation device 1030 contacts the skin of the subject, the display can show a visual aid from an imaging device 1036, such as ultrasound array output. As discussed herein, in some embodiments, the robotic arm 1020 can be motorized. In some embodiments, the robotic arm 1020 is configured to support the manipulation device 1030 while a user engages the patient. In some embodiments, the manipulation device 1030 may be counterbalanced to provide better control to the user during the procedure. As seen in FIG. 10F, the manipulation device 1030 may be coupled to the robotic arm 1020 via a ball joint located on an end of the manipulation device 1030 that is opposite to the cartridge 1040 and handle, which provides the caregiver with an ability to provide multi-dimensional rotation of the manipulation device 1030 about the distal end of the robotic arm 1020 as discussed below.

In some embodiments, the robotic arm 1020 can include sensors to measure force and/or torque in order to perform the medical procedure in a safe manner. For instance, a needle penetration force that is greater than a threshold value can cause damage to the skin, blood vessels, and/or neighboring tissues. Accordingly, measuring the force and/or torque during the medical procedure can ensure the needle penetration force is below the threshold value. For example, for a 25 G needle, the maximum penetration force that can be applied by the needle to puncture a forearm vein is 2.5 N. Similarly, the maximum penetration force can be determined for an 18 G needle, a 22 G needle, etc. If the penetration force measured by the sensors exceeds the identified maximum penetration value, in some embodiments, the system 1000 can be automatically shut down. For example, in response to the penetration force exceeding the penetration value, a control unit (e.g., structurally and/or functionally similar to control unit 405 in FIGS. 4A-4B) can automatically shut down the system. In some embodiments, the measured force can also be an indicator of whether or not the needle may have penetrated into a tissue of the subject. For example, when the needle penetrates the tissue, the penetration force can drop. In such scenarios, a user can be notified via a display (e.g., structurally and/or functionally similar to display 404b in FIG. 4A) that the needle has penetrated the tissue. In some embodiments, the robotic arm 1020 can include collision-sensing skin sensors to identify whether the manipulation device 1030 is in contact with the skin of the subject prior to performing the medical procedure. In some embodiments, the sensors can be used to set a skin position associated with the patient. In some embodiments, after the manipulation device 1030 is positioned as desired, the robotic arm 1020 and/or the manipulation device 1030 can be locked.

FIG. 10F illustrates the vascular access system 1000 being coupled to a cartridge 1040. In some embodiments, the cartridge 1040 can include a needle, a guidewire, and/or a catheter. The cartridge 1040 can be coupled to the manipulation device 1030 when the manipulation device 1030 is positioned as desired to reduce the likelihood of inadvertent needle punctures during positioning of the manipulation device 1030. One of the benefits of the use of a cartridge 1040 with a robotic system is that the caregiver can avoid handling the needle and/or guidewire before, during, and after the procedure as well as avoid handling the catheter before and during the procedure. This touch-free workflow minimizes the risk of bloodstream infections caused by contamination of a device prior to insertion. The touch-free workflow also significantly reduces the risk to caregivers of inadvertent needle sticks.

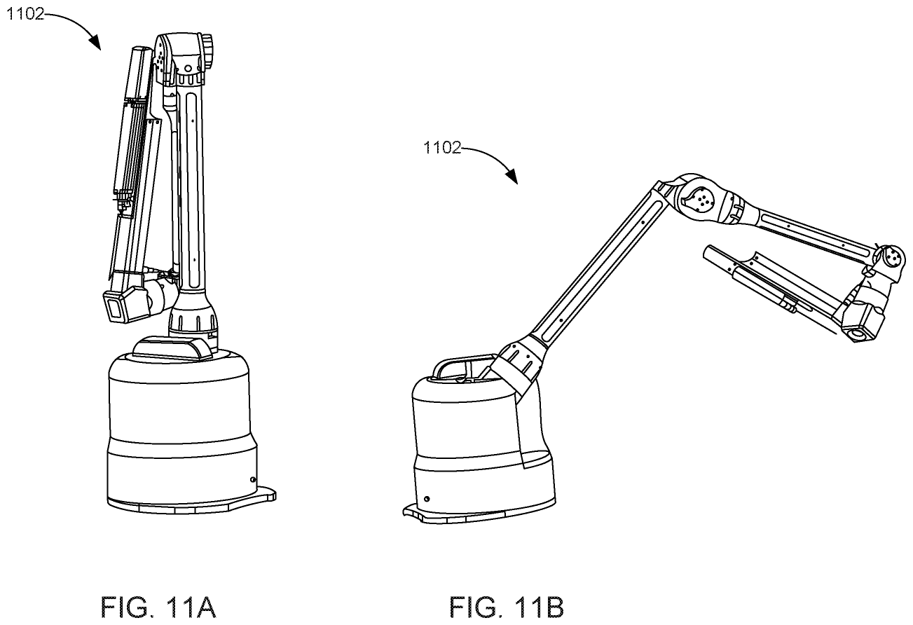

FIG. 11A illustrates a portion of a robotic system 1102 in a first configuration for storage. In the first configuration, the robotic system 1102 is positioned so that the robotic system 1102 folds into a compact shape. The first configuration may reduce the likelihood that the robotic system 1102 is damaged when not in use. In some embodiments, the robotic system 1102 may include a locking mechanism that locks the robotic system 1102 in the first configuration. FIG. 11B illustrates the robotic system 1102 in a second configuration for vascular access. In the second configuration, the robotic arm is deployed and prepared for performing a vascular access procedure.

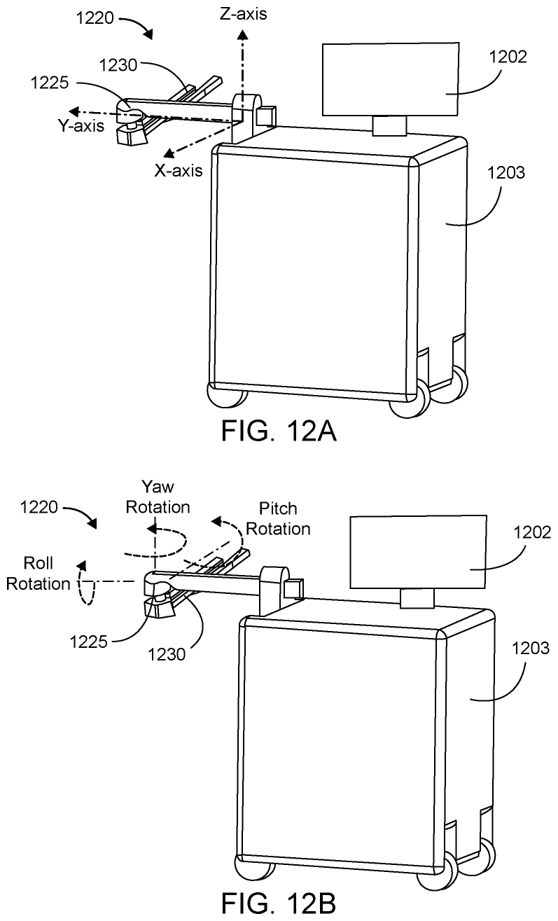

FIG. 12A-12B illustrates another example of a robotic system 1202. The robotic system 1202 includes a base 1203, a robotic arm 1220, and a manipulation device 1230. The robotic arm 1220 is coupled to the base 1203 such that the robotic arm 1220 can translate along an X-axis, a Y-axis, and a Z-axis as seen in FIG. 12A. In some embodiments, the robotic arm 1220 is coupled to the base 1203 to allow for vertical positioning. The position of the robotic arm 1220 can reduce the hanging weight of the robotic arm 1220 and make it more stable during operation. The robotic arm 1220 is coupled to the manipulation device 1230 via a ball joint 1225 that is configured to provide the manipulation device 1230 with yaw rotation, pitch rotation, and roll rotation. The ball joint 1225 can be configured to include a braking system configured to lock the manipulation device 1230 in a specific position and/or orientation.

The translation and rotation of the robotic arm 1220 allow for the manipulation device 1230 to be positioned in space to a desired position and orientation while holding the manipulation device 1230 stationary. In some embodiments, the robotic arm 1220 can be motorized and controlled by a human interface (e.g., a controller, a joystick, etc.). In some embodiments, once the robotic arm 1220 positions the manipulation device 1230 in a desired position, the robotic arm 1220 can be locked into place with integrated joint brakes. In some embodiments, information related to the operation of the robotic arm 1220 is shown on a display.

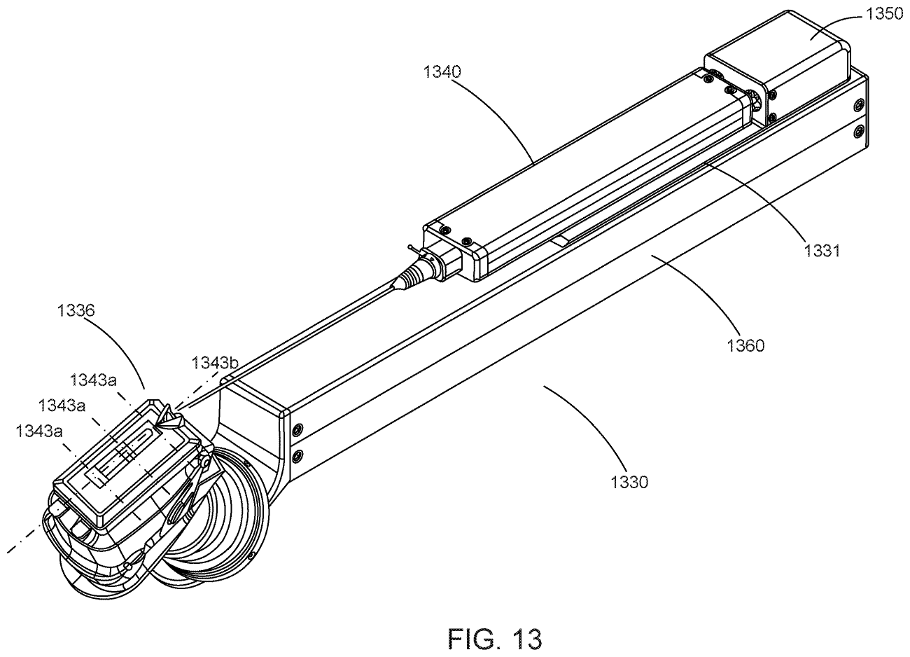

FIG. 13 illustrates a variation of a manipulation device 1330 (e.g., structurally and/or functionally similar to other manipulation devices described herein, including the manipulation device 230 in FIG. 2). The manipulation device 1330 can include a body 1360 and a cartridge motor assembly 1350. The body 1360 and the cartridge motor assembly 1350 provide components that drive the needle, catheter, and/or guidewire of a cartridge assembly 1340. The manipulation device 1330 can receive a cartridge assembly 1340 (e.g., structurally and/or functionally similar to other cartridge assemblies described herein, including the cartridge assembly 540 in FIG. 5). The cartridge assembly 1340 can be attached to the manipulation device 1330 by engaging the cartridge assembly 1340 with the cartridge motor assembly 1350 such that the actuators within the cartridge assembly 1340 are operably coupled to the motors in the cartridge motor assembly 1350. In some embodiments, one or more mechanical engagement features (e.g., adaptor, clutch, etc.) between the cartridge assembly 1340 and the manipulation device 1330 can mechanically couple the cartridge assembly 1340 to the manipulation device 1330. For example, the housing of the cartridge assembly 1340 can include features such as tabs that fit within slots in the housing of the manipulation device 1330. The cartridge assembly 1340 may be coupled to and/or supported by a translation plate 1331 (e.g., similar to other plates or platforms described herein) of the manipulation device 1330. The translation plate 1331 is configured to slide along the body 1360 and translates both the cartridge assembly 1340 and the cartridge motor assembly 1350 during operation.

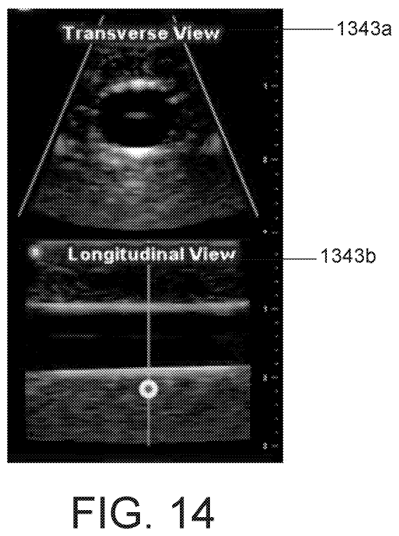

FIG. 13 shows an imaging device 1336 located at a distal end of the manipulation device 1330 that aids the medical caregiver in positioning the manipulation device 1330 with a blood vessel. In some embodiments, the imaging device 1336 can be an ultrasound imaging device with multiple transducers that provide multiple views of the vessel along different planes. For example, the imaging device 1336 can provide views along a longitudinal plane 1343b (i.e., in alignment with a longitudinal axis of the manipulation device 1330), and a transverse plane 1343a (i.e., transverse to the longitudinal axis of the manipulation device). FIG. 14 illustrates examples of a transverse plane 1343a and a longitudinal plane 1343b of a blood vessel as captured by an ultrasound imaging device.

FIG. 14 shows an example of a transverse plane image 1343a comprising a radial cross-section of the blood vessel, represented by the circular/elliptical dark space in the image 1343a. FIG. 14 also shows an example of a longitudinal plane image 1343b comprising an axial cross-section of the blood vessel, represented by the longitudinal dark space in the image 1343b. As discussed herein, when the manipulation device is positioned appropriately, the ultrasound arrays provide multiple images that act as a visual aid to observe the movement of the needle, catheter, and/or guidewire into the tissue and into the blood vessel. Subsequent movement of the manipulation device can be controlled based on the feedback from the visual aid.

As the needle advances into the field of view of the ultrasound array, the tip of the needle may remain in a central longitudinal plane (e.g., longitudinal plane 1343b in FIG. 13) of the ultrasound array and the manipulation device because the ultrasound array and the manipulation device and/or the robotic arm are physically connected to each other. However, as the tip of the needle advances into the blood vessel, the transverse plane of the needle may not remain in the transverse plane (e.g., transverse plane 1343a in FIG. 13) of the ultrasound array.

In some embodiments, a position encoder on the actuator associated with the needle (e.g., device actuator(s) 534 in FIG. 5) and a limit switch can be used to determine the absolute position of the needle tip. The absolute position of the needle tip can be used to determine which transverse plane to display as a visual aid to the user.

Referring generally to FIGS. 15A-15B, imaging devices that include an ultrasound transducer array that is configured to provide ultrasound images along multiple transverse planes and/or multiple longitudinal planes are discussed. Using multiple planes of the same type can allow for determining if an insertion angle of the needle 20 is desirable (e.g., aligned into the length of the blood vessel) while eliminating uncertainty present in single-plane methods. In some embodiments, multiple transverse planes are specifically used for aligning the manipulation device and/or cartridge assembly (and specifically, a trajectory of the catheter, the guidewire, and/or the needle) with the target vessel. Using multiple transverse planes can reduce reliance on user interpretation of longitudinal views (which may not provide a comprehensive view of the vessel) and can simplify targeting the blood vessel to align the expected trajectory with the centroids of the target vessel in the multiple transverse planes.

FIG. 15A illustrates an example of the bottom or patient-contacting side of an imaging device 1536 as described herein. In this variation, the imaging device 1536 is configured to provide up to five ultrasound views along three different transverse planes 1543b and in different positions along a longitudinal plane 1543a. In this variation, the ultrasound array provides a first transverse view T1 that is closest to the needle path N, a second transverse view T2, a third transverse view T3, a first longitudinal view L1, and a second longitudinal view L2. The transverse views T1, T2, T3 can be spaced evenly apart along the transverse planes 1543b, with the longitudinal views L1, L2 extending between the transverse views T1, T2, T3. FIG. 15A also illustrates that views T1 and L1 are closest to the needle 20. Since variations of the system provide for a needle 20 that advances in the longitudinal plane 1543 but at an angle that is fixed relative to the imaging device 1536, the views of interest will vary based on the depth of the vessel. As discussed below, the present system can allow for selection, display, and manipulation of these views to provide improved guidance to the operator by minimizing views that could otherwise be distracting for the operator and highlighting/maximizing views that are immediately important in real-time, given the dynamic movement of the needle, guidewire, and/or catheter during the procedure.

In some embodiments, the transverse views T1, T2, T3 are spaced between about 1 cm and 4 cm apart, inclusive of all ranges and values therebetween. As discussed below, in some variations, not all of the views are shown on a user interface. For example, if the depth of the target vessel is less than about 10 mm (e.g., superficial), only the first transverse view T1, the second transverse view T2, and the first longitudinal view L1 are displayed. If the depth of the target vessel is greater than about 10 mm and less than about 20 mm, the third transverse view T3 and the second longitudinal view L2 are displayed. In some embodiments, the imaging device 1536 may automatically activate the desired views based on the depth of the target vessel. In some embodiments, the depth can be determined automatically and/or input by a user. In some embodiments, the imaging device 1536 can include any number of transverse views and longitudinal views.

The configuration of the imaging device 1536 discussed herein, which is configured to display 5 different views, is for purposes of illustration only. It is contemplated that variations of the system can use an imaging device capable of providing any plurality of views to aid in positioning of the needle, guidewire, and/or catheter in accordance with the methods and techniques discussed herein.

FIG. 15B illustrates an imaging device 1536 and various views T1, T2, T3, L1, and L2 when the imaging device 1536 is located on a body part of a patient and generally aligned with a blood vessel 14 as viewed from a side. As seen in FIG. 15B, the transverse views T1, T2, T3 are spaced along the blood vessel 14 such that the imaging device 1536 provides spaced apart radial cross sections in spaced apart transverse planes 1543 of the blood vessel 14. Longitudinal views L1 and L2 provide axial cross sections along the blood vessel 14 in a longitudinal plane 1543a. As further discussed below, the multiple transverse views can be used to ensure that the needle path 170 is in alignment with the blood vessel 14 so that the needle 20 punctures the blood vessel 14 at an acceptable location. As discussed below, the needle path 170 is electronically/virtually displayed on the various views, and the needle 20 is observable in real-time as it moves along the path 170. Once the needle 20 advances along the path 170 and into the blood vessel 14, the medical caregiver advances a guidewire 22 from the needle 20 and into the blood vessel. In some variations of the system, an angle of the needle path 170 is fixed relative to the imaging device 1536. As a result, because of this fixed angle, the relevant transducer arrays and resulting views are determined by a depth of the vessel. For example, referring to FIG. 15B, if a vessel is shallow, then the views of T1, T2, and L1, located closer to the needle 20, can be important to view when initially piercing the vessel 14. Likewise, if a vessel is located at a greater depth, the other views, T2, T3, L2, which are spaced farther from the needle 20, can be more useful to display to a user since the needle 20 will not intersect the vessel 14 until it is advanced deeper into tissue. As a result, selectively displaying views, as discussed below, can focus the caregiver's attention to pertinent views and increase the efficiency and safety of the procedure.

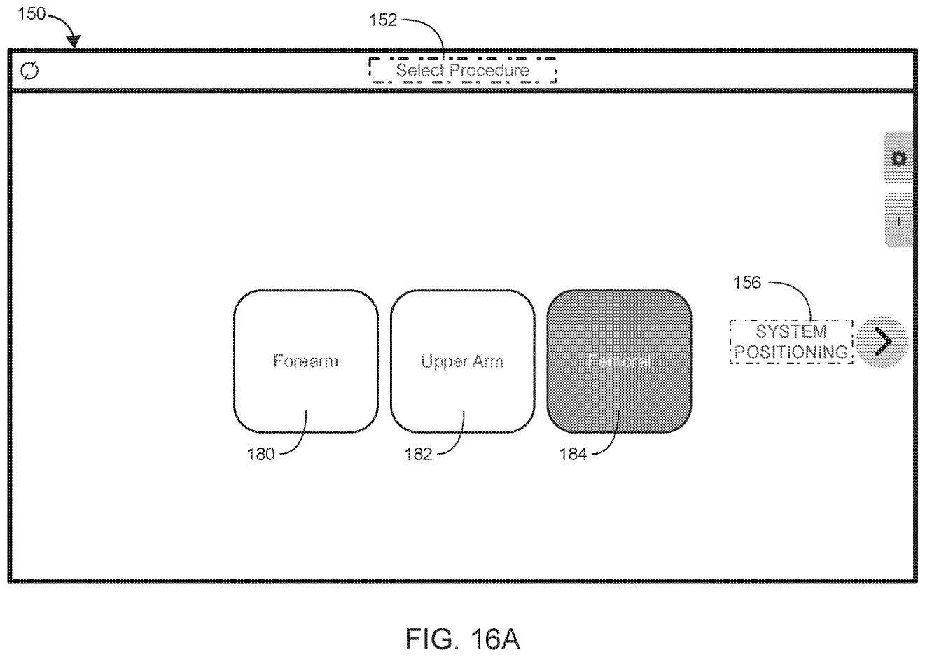

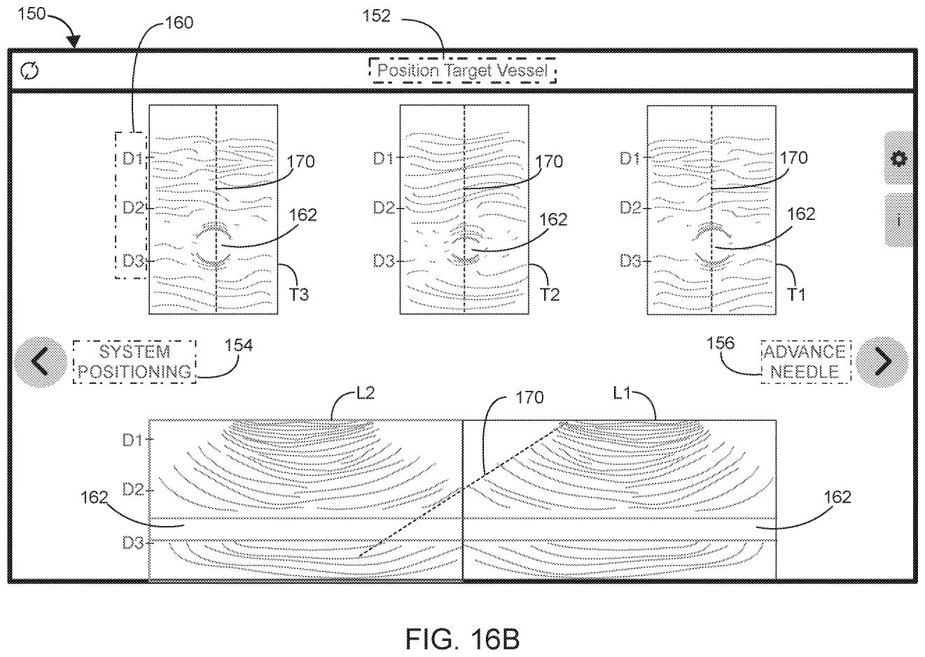

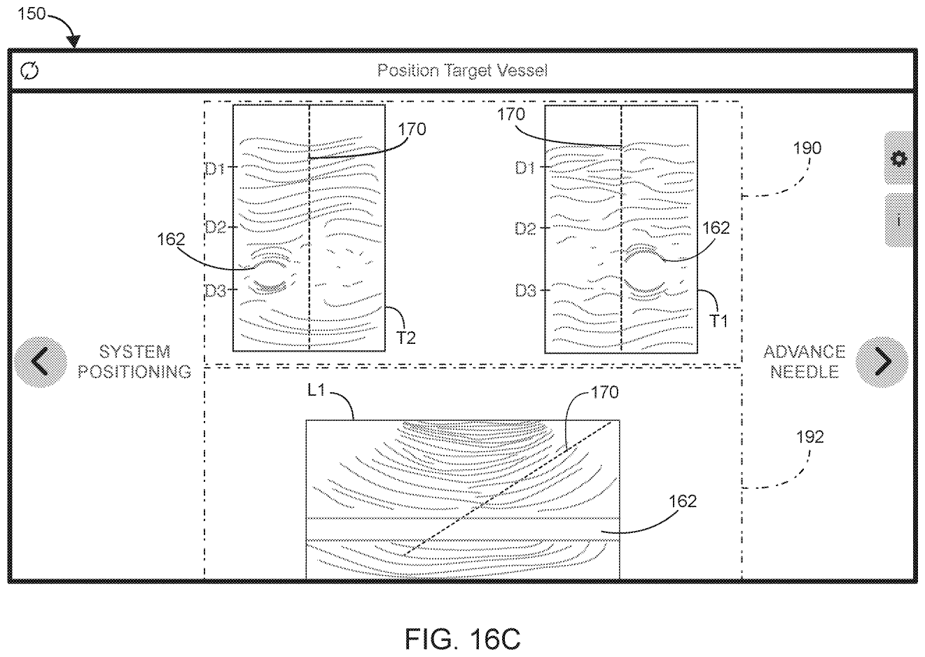

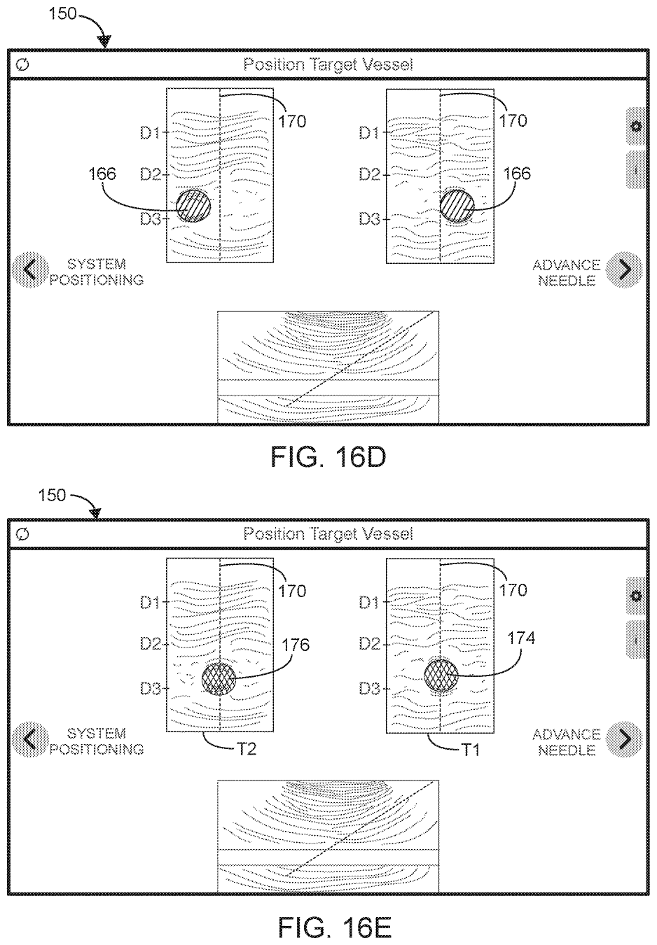

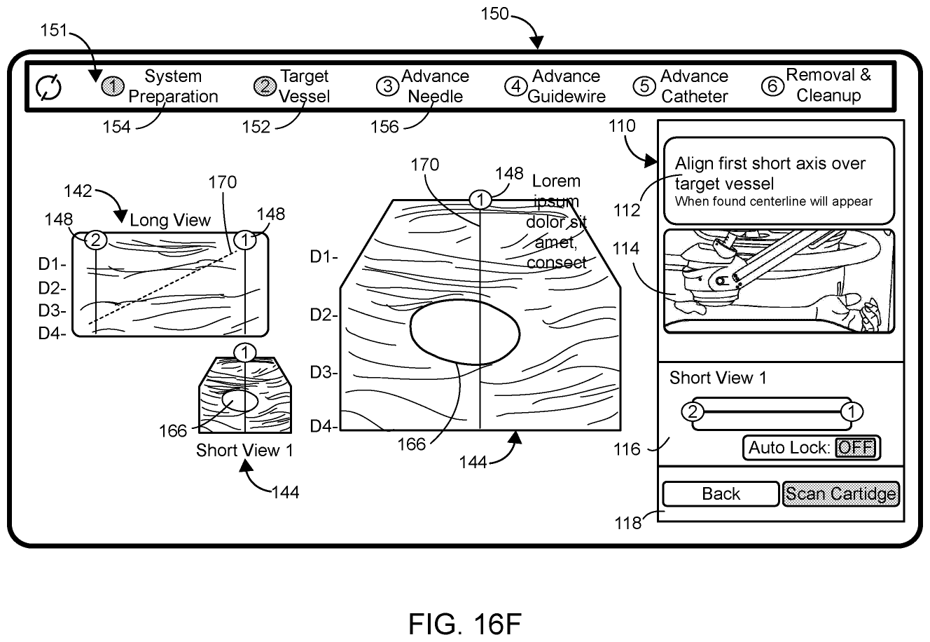

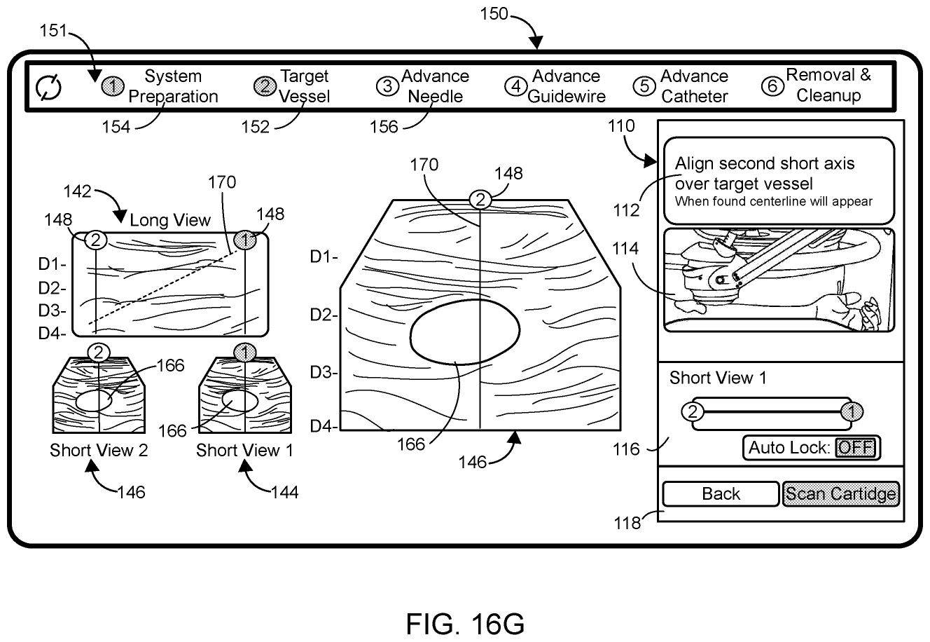

FIG. 16A shows an example of a user interface 150 that can be presented on any system display as described herein. In some variations of the system, the display is a touch-screen display allowing for direct contact of the user interface 150 by the caregiver. FIG. 16 shows an example of one of the initial screens of the user interface 150 where the current process 152 is displayed on the interface 150. In this example, the current process is a “Select Procedure” screen that allows the caregiver to select the intended procedure from a group of procedures 180-184. As discussed herein, the selection of the procedure acknowledges that the vessel will be located at different depths in different body regions and, therefore, the system will show different views or provide different options that are relevant to the particular procedure. The example user interface 150 shown in FIG. 16A shows 3 procedures, “forearm” 180, “upper arm” 182, and “femoral” 184, which represent common regions for catheterization. However, additional variations of the system and user interface 150 can include any number of procedures to account for patient size, body type, arterial placement, venous placement, and/or any other type of characteristic that could be relevant to the procedure. Such information includes, but is not limited to, the skill level of the operator, displaying fewer screens, displaying additional screens, etc. Once a procedure is selected, e.g., FIG. 16A shows “femoral” 184 in a highlighted state; the system can proceed to the next step, either automatically or after a user selects the subsequent step/subprocedure option 156. In this example, the subsequent step/subprocedure 156 is “System Positioning”. Variations of the robotic system can include a user interface that solely controls the system, or that can work in addition to an external control interface, such as that shown in FIG. 21 below. In the latter case, the caregiver can observe information presented by the user interface while operating the system using the external control surface either alone, or in conjunction with the touch-screen display.

FIG. 16B shows a variation of the user interface 150 that displays the various views T1, T2, T3, L1, L2 from the imaging device (not shown in FIG. 16B). As described below, variations of the system and display can include any number of views (more or fewer than T1, T2, T3, L1, L2). FIG. 16B is one example that is intended to convey some of the benefits of the novel system described herein. In the example shown, the user interface 150 displays all views T1, T2, T3, L1, and L2 with a trajectory path 170 that is virtually imposed in the views. The trajectory path 170 represents the path of the needle and/or catheter once they are advanced. The one or more views T1, T2, T3, L1, and L2 can include depth marking indicators 160 to provide the user with information related to the depth of the vessel 162. The trajectory path in views T1-T3 is shown as a straight line, while views L1 and L2 show the trajectory as an angled line. This difference is because views T1-T3 show a radial cross-sectional view of the vessel, while views L1 and L2 show axial cross sections of the vessel.

FIG. 16B also illustrates that the user interface 150 can show various informational prompts. For example, since the catheterization procedure is performed in stages, the user interface 150 displays the current step or subprocedure 152 from the selected procedure and also shows buttons 154, 156 to access a previous step/subprocedure 154 or a subsequent step/subprocedure 156. In the illustrated example, the user interface 150 shows the “Position Target Vessel” step, which is an alignment of the imaging device on the body part so that the trajectory path 170 aligns with the image of the vessel 162. As discussed below, the improved system disclosed herein can selectively display information to focus the caregiver's attention on the most pertinent screen or view, where providing all of the views could overwhelm or distract the caregiver during placement of the needle. In some cases, e.g., placement of a needle in a vessel located deep within the body part (e.g., a femoral vessel), the user interface 150 may display all views.

It is also noted that FIG. 16B illustrates each view as having the trajectory path 170 passing through the imaged representation of the vessel 162. This is intended for explanatory purposes only, since natural anatomic variations may prevent alignment of the trajectory with the representation of the vessel 162 in each view.

FIG. 16C provides an illustration of another user interface 150 screen where the target vessel, as represented by region 162, is located in a region of the anatomy where T1, T2, and L1 provide the most relevant views to assist the caregiver in positioning the manipulation device over the target vessel. Since the caregiver is in the act of positioning the manipulation device, prior to insertion/advancement of a needle, the most pertinent views are the transverse views, e.g., T1 and T2, since these views allow the user to align the needle path 170 with both transverse views, e.g., T1 and T2. As shown in the example of FIG. 16C, view T1 shows the vessel image 162 to the right of the needle path 170, and T2 shows the vessel image 162 to the left of the needle path 170, which indicates that the manipulation device is skewed in relation to the vessel and allows the caregiver to make the appropriate adjustment.

Views T1, T2, and L1 are shown in the various figures for purposes of explanation only. It is understood that any combination of views can be displayed in the user interface 150 as needed to assist in the procedure.

The selection of views can be dependent on the selected procedure (as discussed above). Alternatively, or in combination, the system processor (not shown in FIG. 16C) can use image processing, AI identification, or other means to visually identify the target vessel on the user interface and to also provide the most pertinent views on the user interface 150. In the illustrated example, the system provides views T1 and T2 in a primary focus region 190 of the user interface 150. The primary focus region 190 can optionally increase a size of the views (e.g., T1 and T2), or otherwise visually distinguish selective views, such as by increasing brightness, outlining, colorizing, etc. In contrast, region 192 can be considered a secondary region to provide views (e.g., L1) that will be relevant as the procedure progresses. Again, the views in the secondary region can be scaled to a smaller size than the views in the primary region 190, decreased in brightness, slightly blurred, etc. Positioning of the primary region 190 above the secondary region 192 is one way to maintain the user's focus. It is understood that alternate variations (e.g., positioning the primary region on a side or on the bottom) are within the scope of alternate variations of the user interface 150.

FIG. 16D shows another feature of the system and user interface 150 where the user interface 150 is configured to identify and display a virtual image of a vessel 166. As noted herein, the system can use image processing, artificial intelligence, or other means, as described below, to identify a vessel. In addition, the system can be configured to selectively identify vessels that are suitable for the procedure. In such a case, the system can either identify an undesirable vessel using a specific color or the system can simply not provide a virtual image 166 over the undesired vessel. In addition, the virtual identification 166 of the vessel 166 can include color-based identification depending on whether the system identifies an artery (e.g., red) or a vein (e.g., blue). However, any visual identification can be used. For example, the system and/or user interface can display a cylindrical or elliptical representation 166 with a secondary visual indicator (e.g., an outer ring) that visually identifies (e.g., a separate color) whether the target vessel is desirable for the procedure. In additional variations of the system, the user interface 150 can also optionally prompt the user to ensure that the user intends to perform the procedure on an artery or a vessel, in addition to visually identifying the vessel. In yet additional variations, the system can determine acceptable vessels by filtering for size, depth, adjacent structures, etc. In such cases, the system can only visually identify vessels that pass the screening criteria, such that an operator knows to avoid vessels that may be observable on the images but are not otherwise identified by the virtual features of the user interface.

FIG. 16E is intended to illustrate the user interface 150 when the needle path 170 becomes aligned, or is substantially aligned, with a vessel in one or more views T1, T2. As discussed below, data from the imaging device can be processed to calculate a centroid of the vessel. The system can then determine that the needle path 170 in the transverse views, T1 and/or T2, is sufficiently close to, or intersects the centroid to align the needle path 170 with the vessel. Once sufficient alignment occurs, the user interface 150 can notify the user either through visual, audible, or haptic feedback. For example, as shown in FIG. 16E, the visual indicator 174, 176 changes in appearance to indicate alignment in one or more transverse views T1, T2. While the figure shows that both views, T1, T2, are in alignment, sequential alignment rather than simultaneous alignment typically occurs. In some variations, once one of the views is in alignment, the system will sequentially lock a portion of the arm while the user aligns the remaining view. For example, once T1 or T2 is aligned, the system can lock the robotic arm/manipulation device but allow movement about the ball joint coupled to the manipulation device. In additional variations, the change in appearance of the indicator 174, 176 can be instant (e.g., changing from the indicators shown in FIG. 16D to a green color). Additionally, the indicators 174, 176 can cycle through a progression of visual features to convey that the system is close to alignment (e.g., blue/red-to-yellow-to-green, different shades of one or more colors, etc.). As noted above, the system can also provide audible and/or haptic feedback as the needle path 170 approaches alignment/becomes aligned with the centroid of the vessel. Such haptic feedback can be provided through the touch screen, the manipulation device, and/or the control interface of the manipulation device.

Another optional feature of the system includes locking the robotic arm and/or manipulation device upon securing alignment in the transverse plane. This allows the user to advance to the next stage of advancing the needle. Alternatively, or in combination, when the system identifies the proper vessel, one or more actuators on the robotic arm, and/or manipulation device can self-position the manipulation device to achieve alignment of the needle path 170 with the vessel. In yet another variation, the system can apply a dampening effect for the movement of the robotic arm and/or manipulation device upon approaching alignment. The sequential locking can be user-initiated or automatic by the system.

FIGS. 16F and 16G illustrate another variation of a user interface 150 similar to those discussed herein and configured to provide additional information to assist a caregiver in performing the procedure. As shown, the user interface 150 can display information regarding the various processes 151 and identify the current process 152, previous process 154, and next process 156. The processes 151 can use any type of visual distinctive feature (e.g., coloring, shading, or otherwise distinguishing the number of the process). In the example illustrated in FIG. 16F, the current process is “Target Vessel”, which allows the user to position the robotic assembly in alignment with the vessel.

FIG. 16F also shows a variation of the user interface 150, which includes an information panel 110. The information panel 110 can display any information related to the procedure, such as confirmation of a process, or instructions to start or assist during the process. In the example variation, the information panel 110 includes instructional text 112 and a video or animation 114 illustrating the procedure or positioning of the robotic assembly.

The user interface 150 shown can also adjust views so that the view that is most relevant during the procedure becomes the primary focus (as described above). In this variation, short view 1 or the transverse view 144 is made larger, allowing the view 144 to be the primary focus of the caregiver. Additionally, the longitudinal view or long view 142 can be displayed as shown with axis identifiers 148 that correspond to the axis of the spaced-apart transverse views. FIG. 16F also illustrates a smaller short/transverse view 144 shown below the first short axis from the long view 142. This smaller view can be displayed when positioning the robotic assembly or after the assembly is aligned with the virtual image of the blood vessel 166.

In this variation, the information panel 110 directs the user to align the robotic assembly or imaging device such that the first short axis (which corresponds to the virtual path of the needle 170 in the first transverse view 144) aligns with the vessel. As noted herein, the user interface 150 can display any number of virtual images to assist the caregiver during the procedure. For example, the first transverse view 144 can display a virtual image of a vessel 166 in the first transverse view 144. The first transverse view 144 can also include the virtual needle path or axis 170 along with the axis identifier 148. Once the short axis/needle path 170 aligns with the virtual cross-section of vessel 166 in the first transverse view 144, the user interface 150 can provide visual or other feedback to alert the caregiver.

FIG. 16G shows the state after the first short axis is aligned with the virtual representation of the vessel 166 in the first short view 144. The user interface 150 prompts the caregiver, through the informational panel 110 or other portion of the user interface 150, to align the needle path/axis in the second transverse view 146. As shown, the system can automatically position/scale the second transverse view 146 to be the primary focus of the caregiver. In addition, the system can also show the alignment status of the first short view in any region of the interface 150 (e.g., in the information panel 110, in the long view 142, and/or smaller first transverse view 144, etc.). Accordingly, showing the alignment status of the first transverse view 144 while aligning the second transverse view 146 allows the caregiver to maintain the imaging device's alignment with the vessel's longitudinal axis.

Once the caregiver performs the appropriate positioning of the robotic system and axis of the second short/transverse view 146, the user interface 150 provides visual or other feedback to the caregiver, allowing the caregiver to cause the robotic arms to lock in place. Alternatively, the robotic system can be configured to automatically lock the robotic arm/system in place upon alignment. Once aligned and/or locked, the user interface 150 will proceed to the next process 156, where the options and/or information in the information panel change accordingly.

FIG. 17A illustrates the user interface 150 after the “position target vessel” phase and into the “advance needle phase”. As shown, since the needle path 170 was previously aligned with the vessel, the system adjusts view L1, as discussed above, e.g., in FIG. 16C, to focus the user on a view that aids in the advancement of the needle 20. Accordingly, L1 can be repositioned, scaled, or otherwise highlighted to draw the user's attention. Variations of the system can allow for the additional views, e.g., T1 and T2, to remain on the user interface 150 or simply mask one or more of the additional views. FIG. 17A shows a representation of a needle 20 advanced within tissue for explanatory purposes. The explanation can apply to the state where the needle is not yet advanced into tissue.

FIG. 17A shows the needle 20 advancing along the needle path 170. Typically, and as discussed below, the user advances the needle using the control interface. However, variations of the system allow the user to advance the needle 20 using the user interface 150. During advancement of the needle 20 (and optionally as during advancement of the wire and/or catheter), the user interface 150 can provide a speed/deflection indicator 178 that is intended to provide feedback about the rate of advancement of the needle/wire/catheter. In the illustrated example, the indicator 178 shows directional bars where one bar is highlighted to indicate slow deflection or advancement of the needle 20. If the user operates the control interface, discussed below, to increase deflection/advancement, a second or additional bars will indicate the increased rate of movement. The system can also provide additional progress bars for any part of needle advancement that is not captured or difficult to see on the displayed images.

FIG. 17A also shows an optional “thrust control” 186 that temporarily increases the speed of insertion (i.e., “thrust”) for the needle (or catheter/wire) over a fixed distance or fixed time interval. During advancement of the needle 20 into tissue or through tissue, the needle 20 can cause “tenting” of tissue, which occurs when the needle tip deforms tissue without penetrating the tissue. The ability to provide a thrust option can address this tenting effect since there is less deformation of tissue due to the increased speed. The increased speed allows the needle to overcome tissue resistance prior to deformation as compared to a slower speed. Limiting the distance or time interval over which the needle travels at the increased speed prevents unintended excessive advancement of the needle. In one variation, the user can select the thrust control button 186 on the user interface 150, use a voice-activated command, or (or on any part of the manipulation device), which results in the needle traveling at an increased speed over the fixed distance/time. Afterwards, the speed of the needle reverts to its standard rate of advancement. Variations of the system allow for the user to repeatedly select the thrust control 186 as needed to advance into tissue, through tissue, and/or into the vessel. Similarly, the user control 150 also allows for a vibration control 188, which causes the oscillation or vibration of the needle 20 to overcome tissue resistance. In some variations of the system, vibration of the needle can be combined with the thrust control. Alternatively, or in combination, the system can use machine learning to monitor and recognize the stages of needle advancement and automatically engage the thrust and/or vibration features. For example, the system can detect the needle 20 approaching a surface of the patient's skin and automatically engage the thrust and/or vibration controls 186, 188. Likewise, the system can detect when the needle 20 approaches the vessel wall and automatically engage these features to aid in the puncture of the vessel. Alternatively, the system can rely on machine learning to detect various stages of advancement and suggest engagement of the various features.

The thrust and/or vibration features described above can be combined with automatic force detection where the system detects an increase in motor current or force from the linear actuators and/or a separate force sensor, indicating tissue resistance, and recommends or triggers the thrust and/or vibration features. Alternatively, or in combination, the system can detect a zero point, which is the level of the skin, and could recommend or automate the thrust/vibration features. The system can also use the force detection to confirm that penetration of the skin and/or vessel wall has occurred.

FIGS. 17B to 17D illustrate another feature of the system that relies on machine learning or other image processing to confirm the placement of the needle 20 into a vessel. FIG. 17B shows an example of view L1 showing the needle 20 advanced towards a vessel 162, causing the vessel wall 164 to tent at region 165 (i.e., deflect without being fully penetrated by the needle) inwards. It has been found that sufficient tenting can occur, causing an operator to assume that the needle is positioned within the vessel. Therefore, when the operator advances the wire, the wire fails to enter the vessel. To address this situation, the system uses machine learning, or other modes as discussed herein, to monitor the vessel wall 164 during advancement of the needle 20 into the vessel 162. The system will then monitor the wall 164 of the vessel 162 to detect movement of the wall 164 in the tented region 165 in a reverse direction, as shown by arrows 168. By detecting the recoil 168 of the wall 164, the system can notify the user by providing feedback through visual, audible, and/or tactile feedback. In addition, the system can be configured to alert the user if the recoil 168 is not detected so that the user can confirm whether the needle has penetrated the tissue.

FIGS. 17E and 17F represent additional steps of the catheterization procedure performed using the system. FIG. 17E shows a wire 22 advancing through the needle 20 and into the vessel 162. FIG. 17F shows a state where the system retracts the needle from the vessel 162 and advances a catheter 24 over the wire 22 into the vessel 162. Ultimately, the system retracts the wire from the vessel 162, leaving the catheter 24 positioned within the vessel. It is noted that the system can optionally show all additional views or can rearrange the views in an alternate arrangement than shown in FIGS. 17E and 17F.

Variations of the system and user interface can include a progress indication (e.g., a progress bar), an indication of one or more aspects of the procedure. For example, the progress indication can indicate what stage of the procedure is currently being performed; a progress bar indicating a desired insertion depth, and/or the like. In some embodiments, the progress bar can be associated with the progress of one or more of the device actuators.

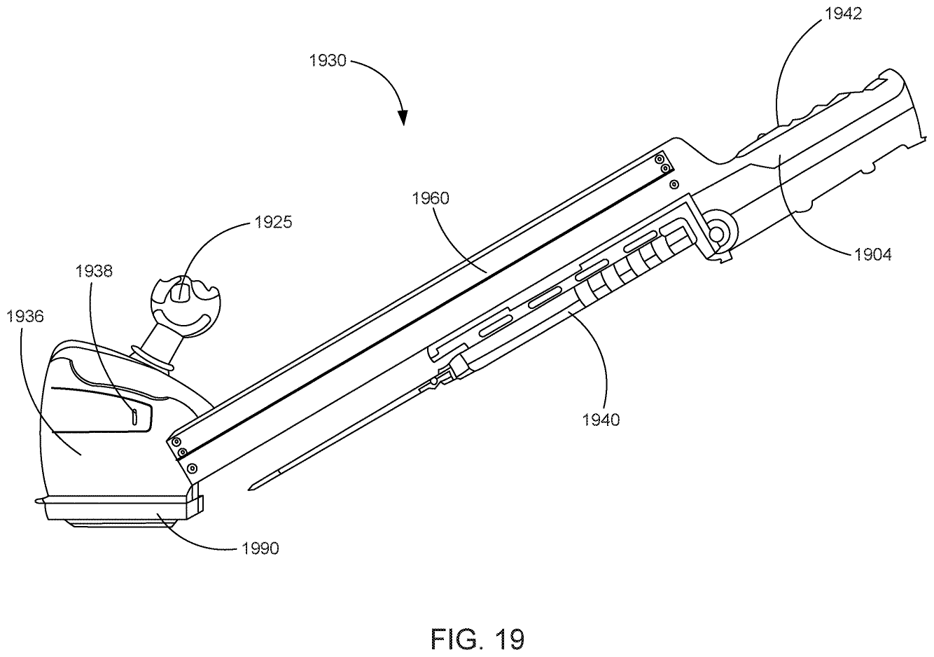

FIG. 19 illustrates a variation of a manipulation device 1930 (as discussed herein) with a ball joint 1925 that is located adjacent to the imaging device 1936 and to the path where the needle, catheter, and guidewire enter into tissue. Positioning of the ball joint 1925 on this distal region of the manipulation device 1930 allows a caregiver to rotate the imaging device 1936 to align with a vessel without having to move the imaging device 1936 through an arc (such as when the joint between the manipulation device 1930 and robotic arm (not shown) are located on a proximal portion of the manipulation device). The design of the housing around the imaging device 1936 is optionally configured to provide an ergonomic shape that allows a caregiver to rest a hand on the body part of the patient while positioning the imaging device 1936 and simultaneously triggering one of the brake controls 1938.

FIG. 19 also shows a variation of a manipulation device 1930 where one brake release control/button/actuator 1938 is located on a first end of the manipulation device 1930 and a second brake release button is located on a control pad 1942 of the manipulation device 1930.

Similar to the manipulation device 1330 of FIG. 13, the manipulation device 1930 includes a body 1960 and a cartridge motor assembly. However, unlike the manipulation device 1330, the cartridge motor assembly can be disposed within a handle 1904 at the proximal end of the manipulation device 1930, and the manipulation device 1930 includes the ball joint 1925 and a drape adaptor 1990. The body 1960 and the cartridge motor assembly provide components that drive the needle, catheter, and/or guidewire. The manipulation device 1930 is coupled to a cartridge assembly 1940 (e.g., structurally and/or functionally similar to other cartridge assemblies described herein, including the cartridge assembly 540 in FIG. 5). The cartridge assembly 1940 can be attached to the manipulation device 1930 by engaging the cartridge assembly 1940 with the cartridge motor assembly 1950 such that the actuators within the cartridge assembly 1940 are operably coupled to the motors in the cartridge motor assembly 1950. In some embodiments, one or more mechanical engagement features (e.g., adaptor, clutch, etc.) between the cartridge assembly 1940 and the manipulation device 1930 can mechanically couple the cartridge assembly 1940 to the manipulation device 1930. For example, the housing of the cartridge assembly 1940 can include features such as tabs that fit within slots in the housing of the manipulation device 1930.

The manipulation device 1930 can include an imaging device 1936 (e.g., structurally and/or functionally similar to imaging device 536 in FIG. 5) that can provide a user with a visual aid of a vascular portion (e.g., blood vessel) as the medical procedure is being performed. In some embodiments, the imaging device 1936 can be an ultrasound imaging device that captures a visual representation of a blood vessel.

The handle 1904 is configured to be held by a user during the operation of the manipulation device 1930. In some embodiments, the handle 1904 can include input devices such as buttons, a joystick, and/or the like. In some embodiments, the input devices can be manipulated by the user to affect one or more operations of the manipulation device 1930. For example, the handle 1904 can include input devices that can cause the manipulation device 1930 to operate the needle, guidewire, and/or the catheter, engage and/or disengage one or more brakes to lock a position, and/or the like. Details associated with the handle are further shown and described in reference to FIG. 21.

The ball joint 1925 is a ball joint configured to allow for the manipulation device 1930 to be rotated about the joint when aligning the manipulation device 1930 with the target structure. In some embodiments, the ball joint 1925 can include a braking system configured to lock the position of the manipulation device 1930. In some embodiments, the manipulation device 1930 may be configured (e.g., counterbalanced) such that the center of mass is between the imaging device 1936 and the ball joint 1925 so that a user does not have to support the weight of the manipulation device 1930 during operation.

The drape adaptor 1990 is positioned at a distal end of the manipulation device 1930 near the distal end imaging device 1936. The drape adaptor is configured to allow for at least a portion of the manipulation device 1930 to be covered with a sanitary drape during a procedure, while allowing for a portion of the imaging device 1936 to be exposed for engagement with the patient's skin. In some embodiments, the drape adaptor can include an opening for the needle, guidewire, and/or the catheter. The drape adaptor is further shown and discussed in reference to FIGS. 20A-20B.

FIGS. 20A-20B illustrate a drape adaptor 2090 similar to the drape adaptors discussed herein. As seen in FIG. 20A, the drape adaptor 2090 is configured to be coupled to a distal end of a manipulation device 2030 and specifically the imaging device 2036.

The drape adaptor 2090 is configured to allow for the imaging device 2036 to engage the patient while draping at least a portion of the manipulation device 2030. The drape adaptor 2090 includes a funnel 2092 configured to aid in directing a needle and catheter to a desired location. As seen in FIG. 20B, the drape adaptor 2090 further includes an opening 2094 which is sized to allow the ultrasound array of the imaging device 2036 to extend through the drape adaptor 2090 to engage the skin of the patient. The drape adaptor 2090 further includes tabs 2096 that can allow for the drape adaptor 2090 to be selectively coupled to the manipulation device 2030. Selective coupling can allow for the drape adaptors 2090 to be replaced between procedures.

FIG. 21 illustrates a control interface 2104 of a manipulation device (e.g., structurally and/or functionally similar to any of the manipulation devices described herein, such as the manipulation device 230 of FIG. 2), in accordance with some embodiments. The control system 2104 can be used to monitor and/or control one or more operations of the manipulation device. The control system 2104 is integrated with the handle of the manipulation device, as described above in reference to FIG. 19. The control system 2104 includes a status identifier 2104a. In some embodiments, the status identifier 2104a can indicate which stage of the procedure is currently selected. For example, the status identifier 2104a can indicate if a needle, a wire, and/or a catheter are currently selected.

The control system 2104 includes a joystick 2104b. The joystick 2104b is configured to operate (e.g., advance, retract, etc.) the selected one of the needle, wire, or the catheter (e.g., as indicated by the status identifier 2104a). The joystick 2104b can have multiple operating speeds. For example, a first operating speed can include advancing and/or retreating at about 1 mm/s, while a second operating speed can be about 4 mm/s. To choose between the needle, wire, and catheter, the control system 2104 includes an advance 2104c and a back 2104d, which can allow the user to choose between the needle, wire, and catheter. At the proximal end of the control system 2104 is a brake release 2104e configured to be actuated when it is desirable to release a locking or brake system of the robotic system. In some embodiments, the brake release 2104e can include two buttons that both need to be actuated (e.g., by both hands of a user) to release the brake system or to permit movement of the robotic system. The two-button configuration can provide a layer of safety as a user is unlikely to press both brake release buttons by accident. The second brake release can be positioned on any portion of the robotic system, including but not limited to the imaging system or a portion of the robotic system that is spaced from the control system 2104. Alternatively, or in combination, one or both of the brake release buttons/switches can be a foot-actuated release button. In some embodiments, the control system 2104 can include an additional input device for operating an all-out condition where it is desirable for the needle, the guidewire, and the catheter to be retracted. In some embodiments, the all-out condition input device can be located so that the input device is unlikely to be accidentally pressed by a user.

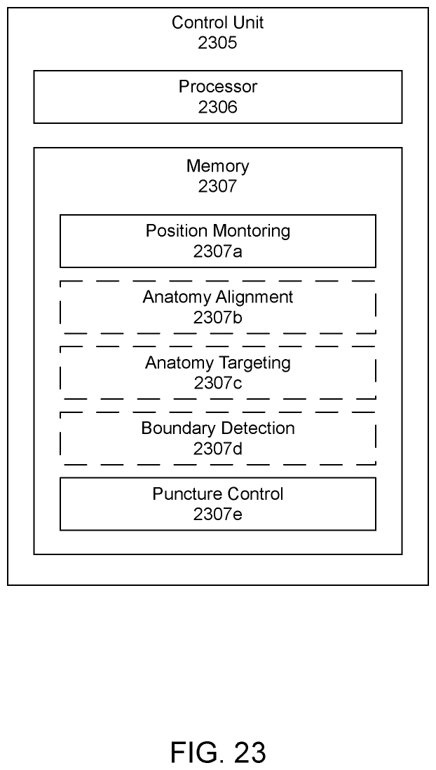

FIG. 23 is a block diagram that illustrates a control unit 2305 of a system for facilitating vascular access (e.g., structurally and/or functionally similar to the system 100 of FIG. 1 and/or any of the systems described herein), in accordance with some embodiments. The control unit 2305 is structurally and/or functionally similar to the control unit 405 of FIGS. 4A-4B. Similar to the control unit 405, the control unit 2305 may be included in a base and can be configured to control and/or monitor one or more components of a robotic system (such as a base, a robotic arm, a manipulation device, the cartridge assembly (e.g., cartridge assembly 240 in FIG. 2 and/or any of the cartridge assemblies described herein), and/or a combination thereof). The control unit 2305 can include any suitable processing device (e.g., processor and/or processing circuitry) configured to run and/or execute functions associated with controlling and/or monitoring one or more components of the robotic system. In some embodiments, the control unit 405 may be communicatively coupled to or include a sensor (e.g., torque sensor, pressure sensors, ammeters, force sensors, position sensors, etc.) configured to monitor the state and/or operation of the robotic system. In some embodiments, the control unit 2305 can be a portion of and/or combined with the control unit 405 so that the resulting functionality is combined.

As seen in FIG. 23, in some embodiments, the control unit 2305 includes a processor 2306 and a memory 2307. The processor 2306 can be a general-purpose processor, microcontroller, a Field Programmable Gate Array (FPGA), an Application Specific Integrated Circuit (ASIC), a Digital Signal Processor (DSP), and/or the like. The memory 2307 can be, for example, a random access memory (RAM), a memory buffer, a hard drive, a flash memory, a database, an erasable programmable read-only memory (EPROM), an electrically erasable read-only memory (EEPROM), a read-only memory (ROM), and/or so forth. In some embodiments, the memory 2307 stores instructions that cause the processor 2306 to execute modules, processes, and/or functions associated with the robotic system. In some instances, the memory 2307 can be operatively coupled to other control units (e.g., such as a control unit in a manipulation device and/or the like).

The memory 2307 can store processor-executable instructions that, when executed by a processor (e.g., processor 2306), cause the processor to implement one or more functions, modules, or processes, such as position monitoring 2307a, anatomy alignment 2307b (optionally), anatomy targeting 2307c (optionally), boundary detection 2307d (optionally), and puncture control 2307e. In some implementations, the memory 2307 can include additional instructions for operating the control unit 2305 and/or instructions for operating the robotic system. As discussed further below, the control unit 2305 is configured to automatically and/or semi-automatically select and align the robotic device with a target vessel. Including automatic and/or semi-automatic targeting and selecting can reduce manual input and reduce the chance of user error while maintaining clinician oversight.

Position monitoring 2307a can include monitoring of one or more positions (e.g., a specific position, relative position, etc.) associated with the patient and/or the robotic system. In some embodiments, position monitoring can be structurally and/or functionally similar to the position monitoring 2307a of FIG. 4B. While the position monitoring 2307a can be configured to monitor the positions of additional components as described in reference to the position monitoring 4207a, the position monitoring 2307a specifically monitors the position of the needle (and specifically the tip of the needle) relative to the target site of the patient. In some embodiments, position monitoring 2307a can include monitoring a position or orientation of the needle relative to a skin surface of the patient. In some embodiments, position monitoring 2307a can include monitoring a position or orientation of the needle relative to a vessel (e.g., an artery) of the patient. In some embodiments, the position monitoring 2307a can include using sensor data to determine the one or more positions. For example, the sensor data can include camera signals, force sensors (e.g., indicating the manipulation device has contacted the patient), motor encoders, and/or the like.

In some embodiments, position monitoring 2307a can include determining a position of the surface of the skin of the patient. For example, the position monitoring 2307a can include determining a zero position of the skin of the patient based on sensors and/or imaging data. For example, the position monitoring 2307a can include determining the zero position of the skin based on the robotic system engaging the patient. As another example, the position monitoring 2307a can include determining a position (e.g., depth below skin zero position, etc.) of the vessel within the patient. In some embodiments, such as when the cartridge is coupled to the manipulation device, the position monitoring 2307a can include monitoring the position of the needle from a zero position (e.g., a fully retracted position). For example, the processor 2306 can be configured to initially zero the location of the needle by activating motor(s) to position the needle at the most proximal position. In other words, the processor 2306 can be configured to activate motor(s) or actuator(s) of the system to set the position of the needle to a zero position (e.g., a predetermined position associated with a starting point of the procedure). In some embodiments, position monitoring 2307a can include monitoring the position of the needle as the needle is advanced into the skin and/or into the vessel. The processor 2306, while the needle is advanced (e.g., to insert the needle into a target vessel and/or skin), can then monitor the position of the needle relative to its zero position.

Anatomy alignment 2307b is functionally and/or structurally similar to the anatomy alignment 407b of FIG. 4B and/or to the method 1700 of FIG. 17, when executed. Similarly, the anatomy alignment 2307b can include receiving information from an imaging and/or sensing device for aligning the system with respect to a target on the patient (e.g., target vessel, target skin position, etc.), or causing a display (e.g., a display associated with an imaging device) to present information to facilitate alignment. For example, the anatomy alignment 2307b can include receiving information associated with aligning a trajectory of the needle with a target vessel. For example, the information can include ultrasound imaging signals corresponding to one or more views (e.g., a transverse view, a longitudinal view, etc.) of the target vessel (e.g., as shown in FIG. 16B). The anatomy alignment 2307b can determine if the manipulation device has been positioned so that the trajectory of the needle is aligned with the target on the patient, e.g., so that the manipulation device can safely advance the needle into the skin and/or the target vessel. For example, the anatomy alignment 2307b can determine if the angle and/or location of the manipulation device is configured to allow the needle to be inserted into the target vessel and/or skin safely (e.g., centrally, at an insertion angle that reduces damage, etc.).

In some embodiments, the anatomy alignment 2307b can automatically determine if a trajectory of the needle is aligned with the target vessel or skin based on one or more sensor inputs. In some embodiments, the processor can cause the robotic system to automatically drive the movement of the robotic arm (e.g., via activating one or more motors of the robotic arm) to reposition the manipulation device and the cartridge to align the needle with the target vessel or the skin. In some embodiments, the anatomy alignment 2307b may be configured to identify walls or boundaries of the target vessel in the images (e.g., using image processing, image segmentation, object recognition, etc.), and determine whether the trajectory of the needle is aligned with the target vessel.

Anatomy targeting 2307c can include identifying a target vessel based on one or more sensor and/or image signals. For example, identifying the target vessel can include identifying a suitable (e.g., desirable) vessel within the patient anatomy. In some embodiments, a suitable vessel may be a vessel in a predetermined size range, a vessel in a predetermined depth range, and/or the like. In some embodiments, the anatomy targeting 2307c can be manually assisted, semi-automatic, or automatic. For example, in some embodiments, a user may provide an input identifying the target vessel. For example, the user can select a target vessel using an input device. In some embodiments, a plurality of vessels can be identified automatically (e.g., via an algorithm, machine learning model, artificial intelligence, and/or the like) and then the user can select the target vessel.

As another example, anatomy targeting 2307c can include automatically identifying a plurality of vessels and then selecting the target vessel. In some embodiments, the anatomy targeting 2307c can include implementing one or more algorithms, machine learning models, and/or the like, e.g., for identifying and then selecting the target vessel. In some embodiments, a computer vision model can be used for monitoring sensor output associated with a blood vessel (and/or an area around the blood vessel) of a patient. In some embodiments, structure recognition methods can be used for automatically identifying and/or detecting the target vessel based on sensor data. Identifying the target vessel automatically can, in some embodiments, minimize user-to-user variability by reducing error from manual processes. In some embodiments, after the anatomy targeting 2307c identifies the target, the user can input a confirmation command. In some embodiments, the output of anatomy targeting 2307c can be used for anatomy alignment 2307b for aligning the needle with the target vessel.

Boundary detection 2307d can include identification of one or more tissue boundaries associated with the target vessel and/or the skin of the patient. The tissue boundary can include skin, vessel walls, and/or the like. In some embodiments, the boundary detection 2307d can include receiving inputs from the anatomy targeting 2307c. For example, imaging data can indicate tissue boundaries associated with the target vessel (e.g., vessel walls). In some embodiments, detecting the tissue boundary can include using one or more sensors associated with the needle to determine if the needle is contacting a tissue boundary. For example, the one or more sensors can include a force sensor configured to measure the force associated with the needle engaging and/or puncturing tissue. As another example, the one or more sensors can include a motor monitoring sensor (e.g., a meter) configured to determine a change in the function of the motor associated with the needle, where the change can be associated with the needle engaging a different (e.g., harder) tissue and/or passing through tissue. In some embodiments, when the boundary detection 2307d determines that a boundary has been identified, the boundary detection 2307d may send a signal indicating that the boundary has been identified. In some embodiments, the signal can indicate if the identified boundary indicates that the needle has engaged tissue or that the needle has passed through tissue.

During operation, the boundary detection 2307d can include determining that the needle has contacted the skin and that a puncture condition is present. The boundary detection 2307d can then generate a signal indicating that a puncture condition is present (e.g., an increase in motor-current draw above a predetermined threshold, an increase in force measurement above a predetermined threshold, etc.). Similarly, the boundary detection 2307d can include determining that the needle has contacted the vessel wall (e.g., an increase in motor-current draw, an increase in force measurement, etc.) and that a puncture condition is present. Again, the boundary detection 2307d can then generate a signal indicating that a puncture condition is present. In some embodiments, the boundary detection 2307d can also determine once the skin and/or the tissue wall is punctured (e.g., decrease in motor-current draw below a predetermined threshold, decrease in force measurement below a predetermined threshold, etc.). The boundary detection 2307d can then generate a signal indicating that a puncture condition is no longer present and/or satisfied.

Puncture control 2307e can include operating the needle to control puncturing through skin and/or the vessel wall of the target vessel. The puncture control 2307e includes operating the needle to puncture at a short, high-velocity, and/or high-acceleration burst to quickly advance the needle through the tissue and to overcome tissue resistance smoothly. The quick advancement is configured to reduce tissue tenting and/or minimize vessel deformation that can be associated with relatively slower tissue puncturing. Further, quick advancement can reduce the likelihood of tissue displacement prior to successful puncture. In some embodiments, the puncturing can be according to one or more predetermined parameters, such as a predetermined distance, a predetermined time, and/or the like. In some embodiments, the predetermined parameters can be constant across patients. In some embodiments, the predetermined parameters can be customized to a patient based on one or more measurements associated with the patient (e.g., vessel depth, diameter, etc.). In some embodiments, the predetermined parameters can be associated with a target vessel type. For example, the predetermined parameters can be different for a forearm vein puncture vs. a femoral artery puncture. In some embodiments, the puncture control 2307e can be used for one or both of puncturing the skin and the target vessel.

In some embodiments, the puncture control 2307e can automatically proceed with a puncture based on the target vessel being identified (e.g., by the anatomy targeting 2307c) and the puncture condition being present. In some embodiments, the puncture control 2307e can proceed with a puncture based on a signal received from a user. In some embodiments, the puncture control 2307e can automatically stop a puncture based on the boundary detection 2307d detecting that a puncture condition is no longer present and/or when the puncture is successful (e.g., skin is punctured, vessel wall is punctured, etc.). In some embodiments, the puncture control 2307e stops the puncture when one or more of the predetermined parameters are satisfied (e.g., after a predetermined distance, after a predetermined amount of time, etc.).

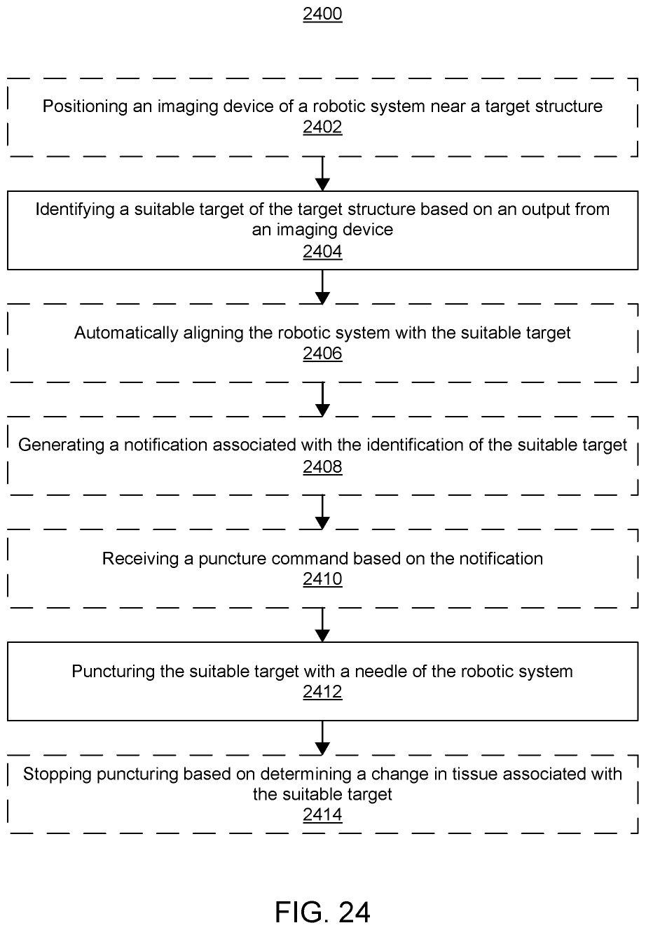

FIG. 24 illustrates a method 2400 for automatically identifying and puncturing target tissue, in accordance with some embodiments. The method 2400 can be executed automatically (e.g., by a control unit such as the control unit 405 of FIGS. 4A-4B and/or the control unit 2305 of FIG. 23) and/or by a user during a vascular access procedure. The method 2400 identifies a suitable target vessel and punctures the suitable target vessel with a needle of a robotic system. In some embodiments, the method 2400 is configured to allow for automatic detection and for automatic puncturing to reduce manual input and to, thus, reduce a likelihood of user error.

At 2402, the method 2400 optionally includes positioning an imaging device of a robotic system near a target structure (e.g., target puncture area, etc.). For example, the robotic system can automatically, or a user can, position the imaging device to substantially engage the skin of the patient near the target structure. In some embodiments, once the robotic device is positioned, an imaging device and/or other sensor can generate signals associated with the target structure. At 2404, the method 2400 includes identifying a suitable target of the target structure based on an output from an imaging device. Identifying the suitable target can include identifying a target vessel (e.g., a suitable vessel) in the target structure as described in anatomy targeting 2307c of FIG. 23. The suitable vessel can be a blood vessel that satisfies predetermined criteria for puncture. In some embodiments, 2404 can be automatic, semi-automatic, and/or manual (e.g., by a user).

At 2406, the method 2400 optionally includes automatically aligning the robotic system with the suitable target. As discussed above with reference to anatomy alignment 2307b, anatomy alignment can include aligning the robotic system for insertion into the skin and the target vessel. In some embodiments, 2406 can be automatic, semi-automatic, and/or manual (e.g., by a user). In some embodiments, aligning can include aligning the needle until a determination is made that a puncture is desired, as described in reference to the boundary detection 2307d of FIG. 23. At 2408, the method 2400 optionally includes generating a notification associated with the identification of the suitable target. In some embodiments, the notification can include that the suitable target has been identified. In some embodiments, the notification can further include whether the robotic system has been aligned for puncture. In some embodiments, the notification can be sent to a user. In some embodiments, the notification is a signal that is configured to automatically trigger puncture. At 2410, the method 2400 optionally includes receiving a puncture command based on the notification. In some embodiments, the puncture command can be included as a signal in the notification. In some embodiments, the puncture command is received from a user indicating that puncture can proceed.

At 2412, the method 2400 includes puncturing the suitable target with a needle of the robotic system. As described above in reference to puncture control 2307e, in some embodiments, the needle punctures the suitable target at a fast velocity and/or fast acceleration to quickly puncture through the skin and/or vessel wall. In some embodiments, the puncture is based on one or more predetermined parameters. In some embodiments, the puncture proceeds until a signal is received, stopping the puncture procedure. In some embodiments, the puncture proceeds according to a combination of commands and/or predetermined parameters. At 2414, the method 2400 optionally includes stopping puncturing based on determining a change in tissue associated with the suitable target. For example, if the boundary detection determines that the puncture is successful, the puncture can be stopped. In some embodiments, the puncturing can be stopped based on one or more predetermined parameters being satisfied. In some embodiments, such as when the method 2400 is used to puncture skin, at least a portion of the method 2400 can repeat to puncture the target vessel.

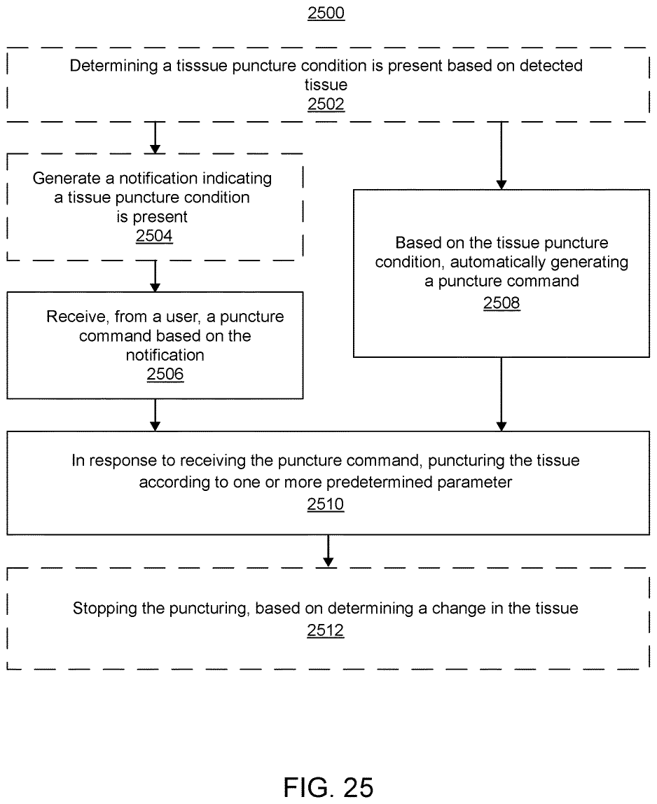

FIG. 25 illustrates a method 2500 for puncturing tissue, in accordance with some embodiments. The method 2500 can be executed automatically (e.g., by a control unit such as the control unit 405 of FIGS. 4A-4B and/or the control unit 2305 of FIG. 23) and/or by a user during a vascular access procedure. The method 2500 manually or automatically generates a puncture command based on a puncture condition being present so that a robotic system can puncture skin or a target vessel. In some embodiments, the method 2500 is configured to allow for automatic detection and for automatic puncturing to reduce manual input and to, thus, reduce the likelihood of user error.

At 2502, the method 2500 optionally includes determining that a tissue puncture condition is present based on detected tissue. For example, and as described in boundary detection 2307d in FIG. 23, a tissue boundary (e.g., skin, vessel wall, etc.) can be detected such that it is desirable to puncture with the needle to reach the desired location within the vessel. For example, an increase in a motor-current measurement or a force measurement associated with the needle can indicate that the needle is in the skin or at the vessel wall. As another example, the position of the vessel wall and/or the skin can be known, and the needle can be within a predetermined puncturing distance of the vessel wall and/or the skin. After 2502, the method 2500 can either continue to 2504 or to 2508. Continuing to 2504 results in a semi-automatic puncturing, while continuing to 2508 results in an automatic puncturing. In some embodiments, a user can choose between automatic and semi-automatic puncturing. In some embodiments, puncturing the skin and the vessel wall can be semi-automatic, automatic, and/or any combination.

At 2504, the method 2500 optionally includes generating a notification indicating that a tissue puncture condition is present. The notification can indicate if the needle is to puncture the skin or the vessel wall. In some embodiments, the notification can be sent to a display for displaying to a user. The user can review the notification to determine if the tissue puncture condition is present or if the notification was a false positive and a puncture is undesirable. If the user determines the condition to be present, the method 2500 continues to 2506. At 2506, the method 2500 includes receiving, from a user, a puncture command based on the notification. In some embodiments, the puncture command can include additional information associated with the puncture, such as parameters and/or the like.

At 2508, the method 2500 includes automatically generating a puncture command based on the tissue puncture condition being present. Automatically generating the puncture command can reduce the amount of input from the user and can reduce the likelihood of user error. In some embodiments, a user can monitor the automatic generation of the puncture command and can provide a stop input in the case of an undesirable puncture. At 2510, and after either 2506 or 2508, the method 2500 includes puncturing the tissue according to one or more predetermined parameters, in response to receiving the puncture command. As described above in reference to puncture control 2307e, in some embodiments, the needle punctures the suitable target at a fast velocity and/or fast acceleration to quickly puncture through the skin and/or vessel wall. In some embodiments, the puncture is based on one or more predetermined parameters. In some embodiments, the puncture proceeds until a signal is received, stopping the puncture procedure. In some embodiments, the puncture proceeds according to a combination of commands and/or predetermined parameters. At 2512, the method 2500 optionally includes stopping the puncturing, based on determining a change in the tissue. For example, if the boundary detection determines that the puncture is successful, the puncture can be stopped. In some embodiments, the puncturing can be stopped based on one or more predetermined parameters being satisfied.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

As used herein, the terms “about” and/or “approximately” when used in conjunction with numerical values and/or ranges generally refer to those numerical values and/or ranges near a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

Also, various inventive concepts may be embodied as one or more methods, of which an example has been provided. The acts performed as part of the method may be ordered in any suitable way. Accordingly, embodiments may be constructed in which acts are performed in an order different from those illustrated, which may include performing some acts simultaneously, even though shown as sequential acts in illustrative embodiments.

Some embodiments and/or methods described herein can be performed by different software (executed on hardware), hardware, or a combination thereof. Hardware modules may include, for example, a general-purpose processor, a field-programmable gate array (FPGA), and/or an application-specific integrated circuit (ASIC). Software modules (executed on hardware) can be expressed in a variety of software languages (e.g., computer code), including C, C++, Java™, Ruby, Visual Basic™, and/or other object-oriented, procedural, or other programming languages and development tools. Examples of computer code include, but are not limited to, micro-code or micro-instructions, machine instructions, such as those produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.), or other suitable programming languages and/or development tools. Additional examples of computer code include, but are not limited to, control signals, encrypted code, and compressed code.

It will be understood that, although the terms first, second, third, etc. may be used herein to describe various elements, these elements should not be limited by these terms. These terms are only used to distinguish one element from another. Thus, a first element discussed below could be termed a second element without departing from the teachings of the present disclosure.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

As for other details of the present invention, materials and manufacturing techniques may be employed as within the level of those with skill in the relevant art. The same may hold true with respect to method-based aspects of the invention in terms of additional acts that are commonly or logically employed. In addition, though the invention has been described in reference to several examples, optionally incorporating various features, the invention is not to be limited to that which is described or indicated as contemplated with respect to each variation of the invention.

Various changes may be made to the invention described and equivalents (whether recited herein or not included for the sake of some brevity) may be substituted without departing from the true spirit and scope of the invention. Also, any optional feature of the inventive variations may be set forth and claimed independently, or in combination with any one or more of the features described herein. Accordingly, the invention contemplates combinations of various aspects of the embodiments or combinations of the embodiments themselves, where possible. Reference to a singular item, includes the possibility that there are plural items of the same type present. More specifically, as used herein and in the appended claims, the singular forms “a,” “an,” “said,” and “the” include plural references unless the context clearly dictates otherwise.

It is important to note that, where possible, aspects of the various described embodiments, or the embodiments themselves can be combined, where such combinations are intended to be within the scope of this disclosure.

1. A robotic system configured to assist a caregiver in a procedure of positioning one or more vascular medical devices into a vessel of a patient, the robotic system comprising:
a control unit in communication with a processor;
an arm member having a first end coupled to a base of the robotic system;
a manipulation device coupled to a second end of the arm member, the manipulation device configured to both retain and selectively advance the one or more vascular medical devices;
an imaging device coupled to the manipulation device opposite to the arm member and configured to obtain a plurality of transverse views and/or a plurality of lateral views of a tissue region of the patient;
where the manipulation device is positionable independently from the base, such that the imaging device can be positioned adjacent to the tissue region;
a video display coupled to the base and in communication with the processor, such that the processor is configured to select a first limited set of one or more of the plurality of transverse views and/or the plurality of lateral views for display on the video display, where the processor selects the first limited set, at least in part, based on a location of the vessel within the tissue region; and
wherein the processor is configured to select a second limited set comprising one or more views from the plurality of transverse views and/or a plurality of lateral views to replace the first limited set for display on the video display, based upon movement of the one or more vascular medical devices relative to the vessel, and where at least one view of the second limited set is different than at least one view of the first limited set;
where the processor is further configured to provide at least one virtual image on the video display over the first limited set or over the second limited set to assist the caregiver during operation of the robotic system to deploy the one or more vascular medical devices.

2. The robotic system of claim 1, wherein the video display comprises a primary focus region configured for displaying the first limited set or the second limited set, and a secondary focus region configured for displaying an alternate view of one of the plurality of transverse views and/or a plurality of lateral views.

3. The robotic system of claim 2, wherein the primary focus region is visually different from the secondary focus region by brightness, outlining, colorizing, scaling, or focus.

4. The robotic system of claim 2, wherein the processor is configured to switch the alternate view into the primary focus region upon advancement of the procedure.

5. The robotic system of claim 1, wherein the at least one virtual image comprises a virtual cross-sectional image of a calculated centroid of a cross-section of the vessel displayed in at least one of the first limited set or the second limited set.

6. The robotic system of claim 5, wherein the at least one virtual image further comprises a virtual path of the one or more vascular medical devices corresponding to an insertion trajectory of the one or more vascular medical devices.

7. The robotic system of claim 6, further comprising changing an appearance of the virtual cross-sectional image when the virtual path intersects the calculated centroid.

8. The robotic system of claim 1, wherein the video display comprises a touch screen and displays one or more touch-screen commands to control the robotic system.

9. The robotic system of claim 8, wherein the one or more touch-screen commands alter an advancement of the one or more vascular medical devices.

10. The robotic system of claim 9, wherein the one or more touch-screen commands alter the advancement of the one or more vascular medical devices by altering a speed or an acceleration of the one or more vascular medical devices in the tissue region over a fixed distance.

11. The robotic system of claim 9, wherein the one or more touch-screen commands alter the advancement of the one or more vascular medical devices by vibrating the one or more vascular medical devices during movement in the tissue region.

12. The robotic system of claim 1, wherein the processor is configured to monitor a wall in the tissue region for tenting, and wherein the processor provides a feedback to the caregiver based on a recoil of a portion of the wall.

13. (canceled)

14. The robotic system of claim 1, wherein the processor alters an advancement of the one or more vascular medical devices by automatically altering a speed or an acceleration of the one or more vascular medical devices in the tissue region over a fixed distance during the procedure.

15.-16. (canceled)

17.-28. (canceled)

29.-30. (canceled)

31. A robotic system configured to assist a caregiver in a procedure of positioning one or more vascular medical devices into a vessel of a patient, the robotic system comprising:
a control unit in communication with a processor;
an arm member having a first end coupled to a base of the robotic system;
a manipulation device coupled to a second end of the arm member, the manipulation device configured to both retain and selectively advance the one or more vascular medical devices;
an imaging device coupled to the manipulation device opposite to the arm member and configured to obtain a plurality of transverse views and/or a plurality of lateral views of a tissue region of the patient;
wherein the manipulation device is positionable independently from the base, such that the imaging device can be positioned adjacent to the tissue region;
a video display coupled to the base and in communication with the processor, such that the processor is configured to select a limited set of one or more of the plurality of transverse views and/or the plurality of lateral views for display on the video display, wherein the processor selects the limited set, at least in part, based on a location of the vessel within the tissue region; and
wherein the processor is configured to change the limited set during the procedure in response to a position of or a change in the one or more vascular medical devices being manipulated by the robotic system;
wherein the processor is further configured to provide at least one virtual image on the limited set to assist the caregiver in operating the robotic system to deploy the one or more vascular medical devices.

32. The robotic system of claim 31, wherein the at least one virtual image comprises a virtual cross-sectional image of a calculated centroid of a cross-section of the vessel displayed in at least one of the limited set.

33. The robotic system of claim 32, wherein the at least one virtual image further comprises a virtual path of the one or more vascular medical devices corresponding to an insertion trajectory of the one or more vascular medical devices.

34. The robotic system of claim 33, further comprising changing an appearance of the virtual cross-sectional image when the virtual path intersects the calculated centroid.

35.-65. (canceled)
